SEQUENCE LISTING (76 sequences)

SEQ ID NO: 1 - DNA, 1494 bp, Homo sapiens
atggccgccgccgccgccgccgcgccgagcggaggaggaggaggaggcgaggaggagagactggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggaaacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagc

SEQ ID NO: 2 - DNA, 2556 bp, Homo sapiens
atgaagcgagcagctgcaaagcatctaatagaacgctactaccaccagttaactgagggctgtggaaatgaagcctgcacgaatgagttttgtgcttcctgtccaacttttcttcgtatggataataatgcagcagctattaaagccctcgagctttataagattaatgcaaaactctgtgatcctcatccctccaagaaaggagcaagctcagcttaccttgagaactcgaaaggtgcccccaacaactcctgctctgagataaaaatgaacaagaaaggcgctagaattgattttaaagatgtgacttacttaacagaagagaaggtatatgaaattcttgaattatgtagagaaagagaggattattcccctttaatccgtgttattggaagagttttttctagtgctgaggcattggtacagagcttccggaaagttaaacaacacaccaaggaagaactgaaatctcttcaagcaaaagatgaagacaaagatgaagatgaaaaggaaaaagctgcatgttctgctgctgctatggaagaagactcagaagcatcttcctcaaggataggtgatagctcacagggagacaacaatttgcaaaaattaggccctgatgatgtgtctgtggatattgatgccattagaagggtctacaccagattgctctctaatgaaaaaattgaaactgcctttctcaatgcacttgtatatttgtcacctaacgtggaatgtgacttgacgtatcacaatgtatactctcgagatcctaattatctgaatttgttcattatcgtaatggagaatagaaatctccacagtcctgaatatctggaaatggctttgccattattttgcaaagcgatgagcaagctaccccttgcagcccaaggaaaactgatcagactgtggtctaaatacaatgcagaccagattcggagaatgatggagacatttcagcaacttattacttataaagtcataagcaatgaatttaacagtcgaaatctagtgaatgatgatgatgccattgttgctgcttcgaagtgcttgaaaatggtttactatgcaaatgtagtgggaggggaagtggacacaaatcacaatgaagaagatgatgaagagcccatccctgagtccagcgagctgacacttcaggaacttttgggagaagaaagaagaaacaagaaaggtcctcgagtggaccccctggaaactgaacttggtgttaaaaccctggattgtcgaaaaccacttatcccttttgaagagtttattaatgaaccactgaatgaggttctagaaatggataaagattatacttttttcaaagtagaaacagagaacaaattctcttttatgacatgtccctttatattgaatgctgtcacaaagaatttgggattatattatgacaatagaattcgcatgtacagtgaacgaagaatcactgttctctacagcttagttcaaggacagcagttgaatccatatttgagactcaaagttagacgtgaccatatcatagatgatgcacttgtccggctagagatgatcgctatggaaaatcctgcagacttgaagaagcagttgtatgtggaatttgaaggagaacaaggagttgatgagggaggtgtttccaaagaattttttcagctggttgtggaggaaatcttcaatccagatattggtatgttcacatacgatgaatctacaaaattgttttggtttaatccatcttcttttgaaactgagggtcagtttactctgattggcatagtactgggtctggctatttacaataactgtatactggatgtacattttcccatggttgtctacaggaagctaatggggaaaaaaggaacttttcgtgacttgggagactctcacccagttctatatcagagtttaaaagatttattggagtatgaagggaatgtggaagatgacatgatgatcactttccagatatcacagacagatctttttggtaacccaatgatgtatgatctaaaggaaaatggtgataaaattccaattacaaatgaaaacaggaaggaatttgtcaatctttattctgactacattctcaataaatcagtagaaaaacagttcaaggcttttcggagaggttttcatatggtgaccaatgaatctcccttaaagtacttattcagaccagaagaaattgaattgcttatatgtggaagccggaatctagatttccaagcactagaagaaactacagaatatgacggtggctataccagggactctgttctgattagggagttctgggaaatcgttcattcatttacagatgaacagaaaagactcttcttgcagtttacaacgggcacagacagagcacctgtgggaggactaggaaaattaaagatgattatagccaaaaatggcccagacacagaaaggttacctacatctcatacttgctttaatgtgcttttacttccggaatactcaagcaaagaaaaacttaaagagagattgttgaaggccatcacgtatgccaaaggatttggcatgctg

SEQ ID NO: 3 - DNA, 1833 bp, Homo sapiens
atggaggagctggtggtggaagtgcggggctccaatggcgctttctacaaggcatttgtaaaggatgttcatgaagattcaataacagttgcatttgaaaacaactggcagcctgataggcagattccatttcatgatgtcagattcccacctcctgtaggttataataaagatataaatgaaagtgatgaagttgaggtgtattccagagcaaatgaaaaagagccttgctgttggtggttagctaaagtgaggatgataaagggtgagttttatgtgatagaatatgcagcatgtgatgcaacttacaatgaaattgtcacaattgaacgtctaagatctgttaatcccaacaaacctgccacaaaagatactttccataagatcaagctggatgtgccagaagacttacggcaaatgtgtgccaaagaggcggcacataaggattttaaaaaggcagttggtgccttttctgtaacttatgatccagaaaattatcagcttgtcattttgtccatcaatgaagtcacctcaaagcgagcacatatgctgattgacatgcactttcggagtctgcgcactaagttgtctctgataatgagaaatgaagaagctagtaagcagctggagagttcaaggcagcttgcctcgagatttcatgaacagtttatcgtaagagaagatctgatgggtctagctattggtactcatggtgctaatattcagcaagctagaaaagtacctggggtcactgctattgatctagatgaagatacctgcacatttcatatttatggagaggatcaggatgcagtgaaaaaagctagaagttttctcgaatttgctgaagatgtaatacaagttccaaggaacttagtaggcaaagtaataggaaaaaatggaaagctgattcaggagattgtggacaagtcaggagttgtgagggtgaggattgaggctgaaaatgagaaaaatgttccacaagaagaggaaattatgccaccaaattcccttccttccaataattcaagggttggacctaatgccccagaagaaaaaaaacatttagatataaaggaaaacagcacccatttttctcaacctaacagtacaaaagtccagaggggtatggtaccatttgtttttgtgggaacaaaggacagcatcgctaatgccactgttcttttggattatcacctgaactatttaaaggaagtagaccagttgcgtttggagagattacaaattgatgagcagttgcgacagattggagctagttctagaccaccaccaaatcgtacagataaggaaaaaagctatgtgactgatgatggtcaaggaatgggtcgaggtagtagaccttacagaaatagggggcacggcagacgcggtcctggatatacttcaggaactaattctgaagcatcaaatgcttctgaaacagaatctgaccacagagatgaactcagtgattggtcattagctccaacagaggaagagagggagagcttcctgcgcagaggagatggacggcggcgtggagggggaggaagaggacaaggaggaagaggacgtggaggaggcttcaaaggaaacgacgatcactcccgaacagataatcgtccacgtaatccaagagaggctaaaggaagaacaacagatggatcgcttcagatcagagttgactgcaataatgaaaggagtgtccacactaaaacattacagaatacctccagtgaaggtagtcggctgcgcacgggtaaagatcgtaaccagaagaaagagaagccagacagcgtggatggtcagcaaccactcgtgaatggagtaccc

SEQ ID NO: 4 - DNA, 3924 bp, Homo sapiens
atgagcaggagccgagccagcatacatagagggagcatcccagctatgagttacgcaccatttcgggatgtccgcgggcccagtatgcaccgaactcaatacgtgcactccccatatgaccgaccaggatggaaccctaggttttgtatcatatctggcaaccaactgctcatgctcgacgaagatgagatccacccactcttgataagggaccgaagatccgaatctagcagaaacaagctcttgcgaaggaccgtcagtgttccagtggaaggacggccccatggagaacacgagtaccatttgggtcggagcagaaggaaaagcgtgccaggaggtaagcaatacagtatggaaggtgccccagccgcaccatttaggcccagtcagggtttcttgagtcggcgccttaagtccagcataaaacggacaaagtcccagcccaaactcgatcgcaccagtagcttccgccagatactcccacgatttcgctccgcagatcacgatagggctaggttgatgcaatccttcaaagaatctcactcacatgagtcactgcttagcccctccagcgcagcagaagctctggagcttaacctcgatgaggattctataatcaagcccgttcattcaagcatcctgggtcaagagttctgtttcgaagttactacaagcagtgggactaagtgtttcgcctgcaggtcagccgccgagcgcgataagtggatcgaaaaccttcagcgggccgttaaaccaaacaaggacaattctaggagggtggataacgtacttaaattgtggataatcgaagctcgcgaactccctcccaagaagagatactactgcgaactttgtctcgacgacatgctgtatgcccgaacaactagtaaaccccgcagtgcctctggagacaccgtgttttggggcgagcacttcgagttcaataacttgcctgccgtcagggctctgagacttcacctttacagggacagtgacaagaagcgaaaaaaagacaaagcagggtatgtgggtcttgtcaccgtaccagttgccacactcgctggacgccacttcaccgaacagtggtaccccgtcacccttcccaccggttccggcggctccggtgggatgggatccgggggaggtggagggtccggaggtggtagcggaggaaaaggcaagggaggttgccccgctgttcggctcaaagcaaggtatcagactatgagtattctgccaatggagctctacaaagagttcgctgagtacgtaacaaatcactatagaatgctctgtgcagtactggaacctgctctcaatgtaaaaggcaaggaagaggtagctagcgcactcgttcacattctgcagtcaactggaaaagcaaaggattttctcagtgacatggctatgagcgaagtggatagattcatggagagagagcatttgatattccgcgaaaacacattggcaaccaaagccatagaagaatacatgagactgatagggcaaaaatatctcaaggatgccataggagaatttatacgcgccctctatgaaagtgaggaaaattgtgaggttgatcccataaagtgcacagcatcatctctggcagagcaccaggccaatctgcgaatgtgctgtgagctggcactctgcaaggtcgtaaacagccactgtgtctttcctcgcgaactgaaggaagtttttgcttcctggcgcttgcggtgcgctgaacggggtcgcgaggacatagccgaccgactcatctctgccagtttgtttttgaggttcctctgtcctgccatcatgtctccctccctctttggcctcatgcaggagtatcccgacgaacaaacttcaagaacattgaccctcattgctaaagtgatccagaaccttgctaatttttctaaattcacttcaaaggaggatttcttgggatttatgaacgaattcttggaactggaatgggggagcatgcaacaatttctttacgagattagcaaccttgatactttgactaacagcagcagtttcgaaggctatattgatttgggccgggagctctcaacccttcatgccctcctctgggaagttcttcctcagctttccaaggaagcacttcttaagttgggtcccctcccacgccttttgaacgacatatctactgcccttcgaaatcccaatattcaacgccagccttctcgacagtccgaacgcccccgcccccagcccgtcgtcctcagagggcccagtgccgaaatgcaaggatatatgatgcgagacctgaactcttcaatagaccttcagtcttttatggctcgcggtctgaatagttctatggatatggccagacttccttcccctactaaagagaaacctccacctccccctccaggagggggtaaggacctgttctatgtatcaagacccccactggcccgctcctcacctgcatattgtacatccagctccgacataactgaacccgagcaaaaaatgcttagtgtgaacaaaagcgtcagtatgcttgaccttcagggtgacggacctggaggaaggcttaacagttccagtgtatccaatctggctgcagtaggcgatctgctgcacagtagccaagcctcccttaccgcagctcttggtctcaggcccgcacccgctggacgcctgtcacagggctcagggtccagcatcaccgcagctggtatgaggctctcccaaatgggggtcaccacagacggcgtccctgcacagcaactccgcattcctctttccttccaaaacccactttttcacatggcagctgacggtcctggtcccccaggaggtcacggtgggggcggcggacacgggccaccctcaagccaccaccatcatcaccaccatcaccatcacagggggggagaacctcctggggacaccttcgctccctttcacggttactcaaaatctgaggatttgtcaagtggagttcccaagccacctgctgcaagcatcttgcatagtcacagctattcagatgagttcgggccctctggaaccgactttactcgcaggcagttgtcacttcaggataatttgcagcatatgctctctccaccccaaatcacaattgggccccagaggcccgcaccaagcggccctggaggtgggtccggtgggggcagcggtggcggaggcggaggacaacctcctccacttcaaagaggtaagtcccagcaactcacagtcagtgctgctcaaaagccaagacccagctctggcaaccttctccagagcccagagcccagttacgggcctgccagaccacggcaacagagcctgtctaaagaaggcagtataggcggttctggggggagcggaggtgggggagggggtggcctcaaaccaagtatcaccaagcagcatagtcagacacccagcacattgaatcctaccatgcctgcttccgagagaacagttgcttgggtctctaatatgccacatctcagtgcagatatcgagagtgctcacatcgagagggaggaatacaaactgaaagagtactcaaagtctatggatgaaagtcgcctcgacagggtcaaggagtacgaagaggaaatacactctctgaaggaacgactgcacatgtccaatcggaagttggaagaatatgagagaagattgttgagccaagaggaacaaacttcaaaaattttgatgcaataccaagcaaggttggaacagagcgaaaagcggttgcgacaacagcaggccgaaaaagactcccagattaagtcaatcatcggacgccttatgctggtagaagaagagctgcgccgggaccatcccgcaatggctgagccacttcccgagccaaaaaaaagactcttggacgctcagcgggggtcattccctccctgggttcagcagaccagggtg

SEQ ID NO: 5 - DNA, 943 bp, Homo sapiens
gtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcag

SEQ ID NO: 6 - DNA, 119 bp, Artificial Sequence
  MINIX artificial intron
gtaagagcctagcatgtagaactggttacctgcagcccaagcttgctgcacgtctagggctcaccgggtttccttgatgaggtaccgacatacttatcctgtcccttttttttccacag

SEQ ID NO: 7 - DNA, 21 bp, Phonitus pyralis
aacgatatgggctgaatacaa

SEQ ID NO: 8 - DNA, 21 bp, Artificial Sequence
  ffluc1_full_miRNA_sequence
aacgatatgggctgaatacaa

SEQ ID NO: 9 - DNA, 321 bp, Phonitus pyralis
tgtttgaatgaggcttcagtactttacagaatcgttgcctgcacatcttggaaacacttgctgggattacttcgacttcttaacccaacagaaggctcgagaaggtatattgctgttgacagtgagcgaaacgatatgggctgaatacaatagtgaagccacagatgtattgtattcagcccatatcgttgtgcctactgcctcggacttcaaggggctagaattcgagcaattatcttgtttactaaaactgaataccttgctatctctttgatacatttttacaaagctgaattaaaatggtataaattaaatcacttt

SEQ ID NO: 10 - DNA, 321 bp, Phonitus pyralis
tgtttgaatgaggcttcagtactttacagaatcgttgcctgcacatcttggaaacacttgctgggattacttcgacttcttaacccaacagaaggctcgagaaggtatattgctgttgacagtgagcgaaacgtgaattgctcaacagtatagtgaagccacagatgtatactgttgagcaattcacgttgtgcctactgcctcggacttcaaggggctagaattcgagcaattatcttgtttactaaaactgaataccttgctatctctttgatacatttttacaaagctgaattaaaatggtataaattaaatcacttt

SEQ ID NO: 11 - DNA, 321 bp, Phonitus pyralis
tgtttgaatgaggcttcagtactttacagaatcgttgcctgcacatcttggaaacacttgctgggattacttcgacttcttaacccaacagaaggctcgagaaggtatattgctgttgacagtgagcgacccgacgatgacgccggtgaatagtgaagccacagatgtattcaccggcgtcatcgtcggggtgcctactgcctcggacttcaaggggctagaattcgagcaattatcttgtttactaaaactgaataccttgctatctctttgatacatttttacaaagctgaattaaaatggtataaattaaatcacttt

SEQ ID NO: 12 - DNA, 321 bp, Phonitus pyralis
tgtttgaatgaggcttcagtactttacagaatcgttgcctgcacatcttggaaacacttgctgggattacttcgacttcttaacccaacagaaggctcgagaaggtatattgctgttgacagtgagcgattcgatagggacaagacaatttagtgaagccacagatgtaaattgtcttgtccctatcgaagtgcctactgcctcggacttcaaggggctagaattcgagcaattatcttgtttactaaaactgaataccttgctatctctttgatacatttttacaaagctgaattaaaatggtataaattaaatcacttt

SEQ ID NO: 13 - DNA, 321 bp, Artificial Sequence
  novel seq1
tgtttgaatgaggcttcagtactttacagaatcgttgcctgcacatcttggaaacacttgctgggattacttcgacttcttaacccaacagaaggctcgagaaggtatattgctgttgacagtgagcgagcatgttacgggacttcttattagtgaagccacagatgtaataagaagtcccgtaacatgcgtgcctactgcctcggacttcaaggggctagaattcgagcaattatcttgtttactaaaactgaataccttgctatctctttgatacatttttacaaagctgaattaaaatggtataaattaaatcacttt

SEQ ID NO: 14 - DNA, 321 bp, Artificial Sequence
  novel seq2
tgtttgaatgaggcttcagtactttacagaatcgttgcctgcacatcttggaaacacttgctgggattacttcgacttcttaacccaacagaaggctcgagaaggtatattgctgttgacagtgagcgagtgaggagcagcggatcttaatagtgaagccacagatgtattaagatccgctgctcctcacgtgcctactgcctcggacttcaaggggctagaattcgagcaattatcttgtttactaaaactgaataccttgctatctctttgatacatttttacaaagctgaattaaaatggtataaattaaatcacttt

SEQ ID NO: 15 - DNA, 321 bp, Artificial Sequence
  novel seq3
tgtttgaatgaggcttcagtactttacagaatcgttgcctgcacatcttggaaacacttgctgggattacttcgacttcttaacccaacagaaggctcgagaaggtatattgctgttgacagtgagcgagtcatgtcgtcacggaacttatagtgaagccacagatgtataagttccgtgacgacatgacgtgcctactgcctcggacttcaaggggctagaattcgagcaattatcttgtttactaaaactgaataccttgctatctctttgatacatttttacaaagctgaattaaaatggtataaattaaatcacttt

SEQ ID NO: 16 - DNA, 321 bp, Artificial Sequence
  novel seq4
tgtttgaatgaggcttcagtactttacagaatcgttgcctgcacatcttggaaacacttgctgggattacttcgacttcttaacccaacagaaggctcgagaaggtatattgctgttgacagtgagcgagagaagtgcggatttcgtatttagtgaagccacagatgtaaatacgaaatccgcacttctcgtgcctactgcctcggacttcaaggggctagaattcgagcaattatcttgtttactaaaactgaataccttgctatctctttgatacatttttacaaagctgaattaaaatggtataaattaaatcacttt

SEQ ID NO: 17 - DNA, 321 bp, Artificial Sequence
  novel seq5
tgtttgaatgaggcttcagtactttacagaatcgttgcctgcacatcttggaaacacttgctgggattacttcgacttcttaacccaacagaaggctcgagaaggtatattgctgttgacagtgagcgaaagcgccaaaggagtctgtgatagtgaagccacagatgtatcacagactcctttggcgcttgtgcctactgcctcggacttcaaggggctagaattcgagcaattatcttgtttactaaaactgaataccttgctatctctttgatacatttttacaaagctgaattaaaatggtataaattaaatcacttt

SEQ ID NO: 18 - DNA, 321 bp, Artificial Sequence
  novel seq6
tgtttgaatgaggcttcagtactttacagaatcgttgcctgcacatcttggaaacacttgctgggattacttcgacttcttaacccaacagaaggctcgagaaggtatattgctgttgacagtgagcgaaagtgcggatttcgtatttgctagtgaagccacagatgtagcaaatacgaaatccgcacttgtgcctactgcctcggacttcaaggggctagaattcgagcaattatcttgtttactaaaactgaataccttgctatctctttgatacatttttacaaagctgaattaaaatggtataaattaaatcacttt

SEQ ID NO: 19 - DNA, 321 bp, Artificial Sequence
  novel seq7
tgtttgaatgaggcttcagtactttacagaatcgttgcctgcacatcttggaaacacttgctgggattacttcgacttcttaacccaacagaaggctcgagaaggtatattgctgttgacagtgagcgaaagtggatgcgatgcgattgctagtgaagccacagatgtagcaatcgcatcgcatccacttgtgcctactgcctcggacttcaaggggctagaattcgagcaattatcttgtttactaaaactgaataccttgctatctctttgatacatttttacaaagctgaattaaaatggtataaattaaatcacttt

SEQ ID NO: 20 - DNA, 321 bp, Artificial Sequence
  novel Seq8
tgtttgaatgaggcttcagtactttacagaatcgttgcctgcacatcttggaaacacttgctgggattacttcgacttcttaacccaacagaaggctcgagaaggtatattgctgttgacagtgagcgaaacggtatccgcaacttgcgatagtgaagccacagatgtatcgcaagttgcggataccgttgtgcctactgcctcggacttcaaggggctagaattcgagcaattatcttgtttactaaaactgaataccttgctatctctttgatacatttttacaaagctgaattaaaatggtataaattaaatcacttt

SEQ ID NO: 21 - DNA, 321 bp, Homo sapiens
tgtttgaatgaggcttcagtactttacagaatcgttgcctgcacatcttggaaacacttgctgggattacttcgacttcttaacccaacagaaggctcgagaaggtatattgctgttgacagtgagcgaaagtacgatgtttacttgatctagtgaagccacagatgtagatcaagtaaacatcgtacttgtgcctactgcctcggacttcaaggggctagaattcgagcaattatcttgtttactaaaactgaataccttgctatctctttgatacatttttacaaagctgaattaaaatggtataaattaaatcacttt

SEQ ID NO: 22 - DNA, 321 bp, Homo sapiens
tgtttgaatgaggcttcagtactttacagaatcgttgcctgcacatcttggaaacacttgctgggattacttcgacttcttaacccaacagaaggctcgagaaggtatattgctgttgacagtgagcgaaagccgctcttggtctctacctagtgaagccacagatgtaggtagagaccaagagcggcttgtgcctactgcctcggacttcaaggggctagaattcgagcaattatcttgtttactaaaactgaataccttgctatctctttgatacatttttacaaagctgaattaaaatggtataaattaaatcacttt

SEQ ID NO: 23 - DNA, 321 bp, Homo sapiens
tgtttgaatgaggcttcagtactttacagaatcgttgcctgcacatcttggaaacacttgctgggattacttcgacttcttaacccaacagaaggctcgagaaggtatattgctgttgacagtgagcgaaagtccgaagatcaagacctgtagtgaagccacagatgtacaggtcttgatcttcggacttgtgcctactgcctcggacttcaaggggctagaattcgagcaattatcttgtttactaaaactgaataccttgctatctctttgatacatttttacaaagctgaattaaaatggtataaattaaatcacttt

SEQ ID NO: 24 - DNA, 321 bp, Homo sapiens
tgtttgaatgaggcttcagtactttacagaatcgttgcctgcacatcttggaaacacttgctgggattacttcgacttcttaacccaacagaaggctcgagaaggtatattgctgttgacagtgagcgagagtccagtatacgcagtgtatagtgaagccacagatgtatacactgcgtatactggactcgtgcctactgcctcggacttcaaggggctagaattcgagcaattatcttgtttactaaaactgaataccttgctatctctttgatacatttttacaaagctgaattaaaatggtataaattaaatcacttt

SEQ ID NO: 25 - DNA, 321 bp, Homo sapiens
tgtttgaatgaggcttcagtactttacagaatcgttgcctgcacatcttggaaacacttgctgggattacttcgacttcttaacccaacagaaggctcgagaaggtatattgctgttgacagtgagcgagtccagtatacgcagtgtacatagtgaagccacagatgtatgtacactgcgtatactggacgtgcctactgcctcggacttcaaggggctagaattcgagcaattatcttgtttactaaaactgaataccttgctatctctttgatacatttttacaaagctgaattaaaatggtataaattaaatcacttt

SEQ ID NO: 26 - DNA, 321 bp, Homo sapiens
tgtttgaatgaggcttcagtactttacagaatcgttgcctgcacatcttggaaacacttgctgggattacttcgacttcttaacccaacagaaggctcgagaaggtatattgctgttgacagtgagcgagactcgggaaaccgttagtattagtgaagccacagatgtaatactaacggtttcccgagtcgtgcctactgcctcggacttcaaggggctagaattcgagcaattatcttgtttactaaaactgaataccttgctatctctttgatacatttttacaaagctgaattaaaatggtataaattaaatcacttt

SEQ ID NO: 27 - DNA, 321 bp, Homo sapiens
tgtttgaatgaggcttcagtactttacagaatcgttgcctgcacatcttggaaacacttgctgggattacttcgacttcttaacccaacagaaggctcgagaaggtatattgctgttgacagtgagcgagcgccttaagtccagcataaatagtgaagccacagatgtatttatgctggacttaaggcgcgtgcctactgcctcggacttcaaggggctagaattcgagcaattatcttgtttactaaaactgaataccttgctatctctttgatacatttttacaaagctgaattaaaatggtataaattaaatcacttt

SEQ ID NO: 28 - DNA, 321 bp, Homo sapiens
tgtttgaatgaggcttcagtactttacagaatcgttgcctgcacatcttggaaacacttgctgggattacttcgacttcttaacccaacagaaggctcgagaaggtatattgctgttgacagtgagcgagagttctgtttcgaagttacttagtgaagccacagatgtaagtaacttcgaaacagaactcgtgcctactgcctcggacttcaaggggctagaattcgagcaattatcttgtttactaaaactgaataccttgctatctctttgatacatttttacaaagctgaattaaaatggtataaattaaatcacttt

SEQ ID NO: 29 - DNA, 321 bp, Homo sapiens
tgtttgaatgaggcttcagtactttacagaatcgttgcctgcacatcttggaaacacttgctgggattacttcgacttcttaacccaacagaaggctcgagaaggtatattgctgttgacagtgagcgaggcgagcacttcgagttcaattagtgaagccacagatgtaattgaactcgaagtgctcgccgtgcctactgcctcggacttcaaggggctagaattcgagcaattatcttgtttactaaaactgaataccttgctatctctttgatacatttttacaaagctgaattaaaatggtataaattaaatcacttt

SEQ ID NO: 30 - DNA, 321 bp, Homo sapiens
tgtttgaatgaggcttcagtactttacagaatcgttgcctgcacatcttggaaacacttgctgggattacttcgacttcttaacccaacagaaggctcgagaaggtatattgctgttgacagtgagcgaaagatccgaatctagcagaaatagtgaagccacagatgtatttctgctagattcggatcttgtgcctactgcctcggacttcaaggggctagaattcgagcaattatcttgtttactaaaactgaataccttgctatctctttgatacatttttacaaagctgaattaaaatggtataaattaaatcacttt

SEQ ID NO: 31 - DNA, 321 bp, Homo sapiens
tgtttgaatgaggcttcagtactttacagaatcgttgcctgcacatcttggaaacacttgctgggattacttcgacttcttaacccaacagaaggctcgagaaggtatattgctgttgacagtgagcgaaaggtcgtaaacagccactgttagtgaagccacagatgtaacagtggctgtttacgaccttgtgcctactgcctcggacttcaaggggctagaattcgagcaattatcttgtttactaaaactgaataccttgctatctctttgatacatttttacaaagctgaattaaaatggtataaattaaatcacttt

SEQ ID NO: 32 - DNA, 321 bp, Homo sapiens
tgtttgaatgaggcttcagtactttacagaatcgttgcctgcacatcttggaaacacttgctgggattacttcgacttcttaacccaacagaaggctcgagaaggtatattgctgttgacagtgagcgaaagaggaaatacactctctgatagtgaagccacagatgtatcagagagtgtatttcctcttgtgcctactgcctcggacttcaaggggctagaattcgagcaattatcttgtttactaaaactgaataccttgctatctctttgatacatttttacaaagctgaattaaaatggtataaattaaatcacttt

SEQ ID NO: 33 - DNA, 35 bp, Phonitus pyralis
gctatgaaacgatatgggctgaatacaaatcacag

SEQ ID NO: 34 - DNA, 105 bp, Phonitus pyralis
gctatgaaacgatatgggctgaatacaaatcacaggctatgaaacgatatgggctgaatacaaatcacaggctatgaaacgatatgggctgaatacaaatcacag

SEQ ID NO: 35 - DNA, 210 bp, Homo sapiens
gctatgaaacgatatgggctgaatacaaatcacaggctatgaaacgatatgggctgaatacaaatcacaggctatgaaacgatatgggctgaatacaaatcacaggctatgaaacgatatgggctgaatacaaatcacaggctatgaaacgatatgggctgaatacaaatcacaggctatgaaacgatatgggctgaatacaaatcacag

SEQ ID NO: 36 - DNA, 105 bp, Homo sapiens
gctatgaaacgatatgggcggaatacaaatcacaggctatgaaacgatatgggcggaatacaaatcacaggctatgaaacgatatgggcggaatacaaatcacag

SEQ ID NO: 37 - DNA, 105 bp, Phonitus pyralis
gctatgaaacgatatgttatgaatacaaatcacaggctatgaaacgatatgttatgaatacaaatcacaggctatgaaacgatatgttatgaatacaaatcacag

SEQ ID NO: 38 - DNA, 105 bp, Phonitus pyralis
gctatgacccaaactgtgaagaatacaaatcacaggctatgagtgtctatcacccgaatacaaatcacaggctatgactaggcccgtttcgaatacaaatcacag

SEQ ID NO: 39 - DNA, 105 bp, Phonitus pyralis
gctatgaaacgatatgcgctgaatacaaatcacaggctatgaaacgatatgcgctgaatacaaatcacaggctatgaaacgatatgcgctgaatacaaatcacag

SEQ ID NO: 40 - DNA, 105 bp, Phonitus pyralis; identical to SEQ ID NO: 34

SEQ ID NO: 41 - DNA, 105 bp, Phonitus pyralis
gctatgaaacgatatgggctcaatacaaatcacaggctatgaaacgatatgggctcaatacaaatcacaggctatgaaacgatatgggctcaatacaaatcacag

SEQ ID NO: 42 - DNA, 105 bp, Phonitus pyralis
gctatgaaacgatatgggctgattacaaatcacaggctatgaaacgatatgggctgattacaaatcacaggctatgaaacgatatgggctgattacaaatcacag

SEQ ID NO: 43 - DNA, 105 bp, Phonitus pyralis
gctatgaaacgatatgggctgaattcaaatcacaggctatgaaacgatatgggctgaattcaaatcacaggctatgaaacgatatgggctgaattcaaatcacag

SEQ ID NO: 44 - DNA, 105 bp, Phonitus pyralis
gctatgaaacgatatgggctgaatactaatcacaggctatgaaacgatatgggctgaatactaatcacaggctatgaaacgatatgggctgaatactaatcacag

SEQ ID NO: 45 - DNA, 105 bp, Phonitus pyralis
gctatgaaacgtgaattgctcaacagtaatcacaggctatgaaacgtgaattgctcaacagtaatcacaggctatgaaacgtgaattgctcaacagtaatcacag

SEQ ID NO: 46 - DNA, 105 bp, Phonitus pyralis
gctatgacccgacgatgacgccggtgaaatcacaggctatgacccgacgatgacgccggtgaaatcacaggctatgacccgacgatgacgccggtgaaatcacag

SEQ ID NO: 47 - DNA, 105 bp, Phonitus pyralis
gctatgattcgatagggacaagacaattatcacaggctatgattcgatagggacaagacaattatcacaggctatgattcgatagggacaagacaattatcacag

SEQ ID NO: 48 - DNA, 105 bp, Artificial Sequence
  Novel seq1 3x binding sites
gctatgagcatgttacgggacttcttatatcacaggctatgagcatgttacgggacttcttatatcacaggctatgagcatgttacgggacttcttatatcacag

SEQ ID NO: 49 - DNA, 105 bp, Artificial Sequence
  Novel seq2 3x binding sites
gctatgagtgaggagcagcggatcttaaatcacaggctatgagtgaggagcagcggatcttaaatcacaggctatgagtgaggagcagcggatcttaaatcacag

SEQ ID NO: 50 - DNA, 105 bp, Artificial Sequence
  Novel seq3 3x binding sites
gctatgagtcatgtcgtcacggaacttaatcacaggctatgagtcatgtcgtcacggaacttaatcacaggctatgagtcatgtcgtcacggaacttaatcacag

SEQ ID NO: 51 - DNA, 105 bp, Artificial Sequence
  Novel seq4 3x binding sites
gctatgagagaagtgcggatttcgtattatcacaggctatgagagaagtgcggatttcgtattatcacaggctatgagagaagtgcggatttcgtattatcacag

SEQ ID NO: 52 - DNA, 105 bp, Artificial Sequence
  Novel seq5 3x binding sites
gctatgaaagcgccaaaggagtctgtgaatcacaggctatgaaagcgccaaaggagtctgtgaatcacaggctatgaaagcgccaaaggagtctgtgaatcacag

SEQ ID NO: 53 - DNA, 105 bp, Artificial Sequence
  Novel seq6 3x binding sites
gctatgaaagtgcggatttcgtatttgcatcacaggctatgaaagtgcggatttcgtatttgcatcacaggctatgaaagtgcggatttcgtatttgcatcacag

SEQ ID NO: 54 - DNA, 105 bp, Artificial Sequence
  Novel seq7 3x binding sites
gctatgaaagtggatgcgatgcgattgcatcacaggctatgaaagtggatgcgatgcgattgcatcacaggctatgaaagtggatgcgatgcgattgcatcacag

SEQ ID NO: 55 - DNA, 105 bp, Artificial Sequence
  Novel seq8 3x binding sites
gctatgaaacggtatccgcaacttgcgaatcacaggctatgaaacggtatccgcaacttgcgaatcacaggctatgaaacggtatccgcaacttgcgaatcacag

SEQ ID NO: 56 - DNA, 21 bp, Artificial Sequence
  MECP2 coding 1 binding site
aagtacgatgtttacttgatc

SEQ ID NO: 57 - DNA, 21 bp, Artificial Sequence
  MECP2 coding 2 binding site
aagccgctcttggtctctacc

SEQ ID NO: 58 - DNA, 21 bp, Artificial Sequence
  MECP2 coding 3 binding site
aagtccgaagatcaagacctg

SEQ ID NO: 59 - DNA, 21 bp, Artificial Sequence
  MECP2 coding 4 binding site
gagtccagtatacgcagtgta

SEQ ID NO: 60 - DNA, 21 bp, Artificial Sequence
  MECP2 coding 5 binding site
gtccagtatacgcagtgtaca

SEQ ID NO: 61 - DNA, 21 bp, Artificial Sequence
  MECP2 coding 6 binding site
gactcgggaaaccgttagtat

SEQ ID NO: 62 - DNA, 21 bp, Artificial Sequence
  SYNGAP1 coding 1 binding site
gcgccttaagtccagcataaa

SEQ ID NO: 63 - DNA, 21 bp, Artificial Sequence
  SYNGAP1 coding 2 binding site
gagttctgtttcgaagttact

SEQ ID NO: 64 - DNA, 21 bp, Artificial Sequence
  SYNGAP1 coding 3 binding site
ggcgagcacttcgagttcaat

SEQ ID NO: 65 - DNA, 21 bp, Artificial Sequence
  SYNGAP1 coding 4 binding site
aagatccgaatctagcagaaa

SEQ ID NO: 66 - DNA, 21 bp, Artificial Sequence
  SYNGAP1 coding 5 binding site
aaggtcgtaaacagccactgt

SEQ ID NO: 67 - DNA, 21 bp, Artificial Sequence
  SYNGAP1 coding 6 binding site
aagaggaaatacactctctga

SEQ ID NO: 68 - DNA, 382 bp, Homo sapiens
agtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 69 - DNA, 164 bp, Artificial Sequence
  Jet promoter
gggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgt

SEQ ID NO: 70 - DNA, 122 bp, Simian virus 40
aacttgtttattgcagcttataatggttacaaataaagcaatagcatcacaaatttcacaaataaagcatttttttcactgcattctagttgtggtttgtccaaactcatcaatgtatctta

SEQ ID NO: 71 - DNA, 225 bp, Bos taurus
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 72 - DNA, 48 bp, Artificial Sequence
  SpA
aataaagagctcagatgcatcgatcagagtgtgttggttttttgtgtg

SEQ ID NO: 73 - DNA, 10 bp, Homo sapiens
gccaccatgg

SEQ ID NO: 74 - DNA, 589 bp, Woodchuck hepatitis virus
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 75 - DNA, 247 bp, Woodchuck hepatitis virus
ataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggt

SEQ ID NO: 76 - DNA, 644 bp, Cytomegalovirus
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

(19) United States
(12) Patent Application Publication
(10) Pub. No.: US 2023/0323391 A1
(43) Pub. Date: Oct. 12, 2023

(54) TRANSGENE EXPRESSION SYSTEM

(71) Applicant: THE UNIVERSITY COURT OF THE UNIVERSITY OF EDINBURGH, Edinburgh, Lothian (GB)

(72) Inventors: Stuart Robert COBB, Edinburgh Lothian (GB); Paul Ross, Edinburgh Lothian (GB); Ralph David HECTOR, Edinburgh Lothian (GB); Susan ROSSER, Edinburgh Lothian (GB); Adam MOL, Edinburgh Lothian (GB)

(21) Appl. No.: 18/003,021

(22) PCT Filed: Jun. 29, 2021
(86) PCT No.: PCT/GB2021/051653

§ 371 Date: Dec. 22, 2022

(30) Foreign Application Priority Data
Jun. 30, 2020 (GB) 2010024.4
Jun. 3, 2021 (GB) 2107990.0

(51) Int. Cl. C12N 15/86; C12N 15/11

(52) U.S. Cl. CPC C12N 15/86; C12N 15/111; C12N 2750/14143; C12N 2330/51; C12N 2310/141; C12N 2320/53; C12N 2830/42; C12N 2830/50; C12N 2830/48

(57) ABSTRACT

A system to limit the expression of a vector-derived transgene within a window that alleviates the disease-causing genetic deficiency without producing overexpression toxicity is described. This provides for ‘dosage-insensitivity’, whereby cells or tissues receiving more vector-derived transgene are disproportionately suppressed through an in-built single gene circuit that can regulate adaptively.

### FIELD OF THE INVENTION

Gene therapy aims to deliver a therapeutic transgene to affect correction in a genetic disease. The present invention provides constructs to generate a relatively fixed level of expression of the transgene across cells receiving different levels of vector-derived transgene. Also described herein is a method of controlling gene expression wherein the control is provided using the described gene circuit.

### BACKGROUND

Whilst the concept of gene therapy to deliver a therapeutic transgene to affect correction in a genetic disease is known, many genes are highly dosage sensitive whereby too little or too much expression of a gene product can have deleterious effects. Viral-mediated gene transfer is a powerful means to deliver therapeutic transgenes to target tissues and cells including cells of the nervous system. High virus titers are typically necessary to enable effective system-wide transduction for maximal therapeutic impact. However, the same high titres may cause overexpression toxicity due to supraphysiological levels of transgene expression achieved in some cells. An effective system is required to limit the expression of the vector-derived transgene within a window that alleviates the disease-causing genetic deficiency without producing overexpression toxicity.

WO2016040395 discusses the use of synthetic RNA circuits for gene transfer. The circuits include a first RNA molecule comprising at least one sequence recognized by a first microRNA specifically expressed in a cell type and a sequence encoding a protein that specifically binds to a RNA motif and inhibits protein production. A first microRNA is described as miR-21. Also provided is a second RNA molecule comprising a sequence recognized by a second microRNA that is not expressed in the cell type at a RNA motif and a sequence encoding an output molecule. A second microRNA is described as miR-141, miR-142 and miR-146. The application describes differential expression of an output protein by different cells (cancer and non-cancer cell) dependent on the endogenous miR provided by these cells.

Strovas T J, Rosenberg A B, Kuypers B E, Muscat R A, Seelig G. MicroRNA-based single-gene circuits buffer protein synthesis rates against perturbations. ACS Synth Biol. 2014; 3(5):324-331 discusses the use of a single-gene microRNA (miRNA)-based feed-forward loop. It provides an intronic miRNA that targets its own transcript. Strovas considers the difficulty of long-term stable expression of engineered genetic programs in mammalian cells. This work utilised a gene circuit in which an intron containing mouse mir-124-3 gene was inserted into a red fluorescent reporter (mCherry). The pre-mRNA is transcribed from a doxycycline inducible promoter leading to a coexpression of the mir-124 and mCherry. A repressive regulatory link between the miRNA and the mCherry transcript was provided by a truncated version of the mir-124-regulated 3′UTR of the Vamp3 gene to the mRNA.

Whilst WO2016040395 discusses the use of differently expressed endogenous miRs, in normal and cancer cells to provide for expression, this use of miRs has limited use in the treatment of non-cancer diseases. The inventors have also determined that the existing methods by Stovas would have multiple off-target effects on a variety of genes that are known to be regulated by endogenous miRNAs such as the miR124 used in this paper. Indeed, miR124 is known to be linked to a number of cancers so would be unsuitable for use in gene therapy. Thus, providing an endogeneous micro RNA may be problematic as endogenous targets in addition to the transgene may be provided.

The present inventors have sought to provide alternative constructs with advantages over the constructs provided in the art.

### SUMMARY OF THE INVENTION

The inventors have determined a system to limit the expression of a vector-derived transgene within a window that alleviates the disease-causing genetic deficiency without producing overexpression toxicity, to enable what the inventors term ‘dosage-insensitivity’, whereby cells or tissues receiving more vector-derived transgene are disproportionately suppressed through an in-built single gene circuit that can regulate adaptively. That is, the vector-derived transgene is downregulated at high vector dosages so that the circuit maintains a relatively stable level of expression across a range of vector doses with the result being that the overall population of cells express a more even and controlled level of vector-derived transgene. Increasing doses of vector will result in more cells expressing the transgene within a cell population but without a concomitant increase in overexpression compared to conventional gene therapy cassettes. Sensitive cell types that often receive high vector loads such as in the heart, liver and dorsal root ganglia will also be less susceptible to superinfection-mediated overexpression by this mechanism.

The present inventors have designed synthetic or non-mammalian miRNA construct(s), which overcome disadvantages associated with mammalian-based miRNA constructs which exhibit the risk of off-target effects. The inventors have demonstrated the utility of non-mammalian or fully synthetic (not known in nature) miRNA to ensure the absence of targets within the host (human genome).

Moreover the inventors have determined how such synthetic components may be used to allow a fine-tuning of the system (number of sites and efficient intron exclusion) to achieve appropriate dosage-insensitivity.

Accordingly a first aspect of the present invention provides a construct comprising:
a promoter;
at least one non-mammalian or synthetic miRNA expressed within an intron, wherein the synthetic miRNA is a sequence which is not naturally occurring;
a transgene;
one or more non-mammalian or synthetic miRNA binding site(s) which provide for control of the expression of the transgene, wherein the synthetic miRNA binding site(s) does not naturally occur; and
a polyadenylation signal.

The miRNA binding sites discussed herein are synthetically derived to differ from mammalian sequences present in a mammalian cell or are provided from another non-mammalian species, for example insect. When the miRNA binding sites are from insect not present in a mammalian sequence, for example ffluc1, non-mammalian systems may be used. The combination of miRNA binding sites and non-mammalian or synthetic miRNA minimise the off-target regulatory effects of the construct. This allows regulation of expression of the transgene to provide a desired dosage (expression) of the transgene.

Suitably the miRNA binding sites which provide for control of the expression of the transgene may be provided within the 3′ UTR, the 5′ UTR and/or within the transgene. Suitably, when provided in the transgene, the miRNA binding site may be codon-optimised such that it provides a synthetic or non-mammalian binding site but does not impact upon the amino acid sequence of the transgene protein. The construct can be used to provide a feed forward loop which allows expression control.

Suitably, a stability element to increase transgene expression may be included. Suitably, the stability element may be located in the 3′ UTR. Suitably this stability element may be the Woodchuck Hepatitis Virus (WHV) Posttranscriptional Regulatory Element (WPRE) (SEQ ID NO: 74). WPRE is a tripartite regulatory element containing gamma, alpha, and beta elements. Suitably, the stability element may be a truncated version of the WPRE, retaining the stability element, but omitting the X-protein sequence, or a ribozyme stability sequence (WPRE3) (SEQ ID NO: 75). WPRE3 is a shortened WPRE sequence containing two of the three regulatory elements of WPRE (a minimal gamma and alpha elements). Suitably, the WPRE3 stability element provides a DNA sequence that creates a tertiary structure in the processed transcript, which enhances transgene expression.

Suitably different promoters can be used with a range of transgenes. In the present invention, the strength of the feed forward loop can be adjusted to allow control of the level of expression of the transgene. This provides for dosage sensitivity. Adjustment of the number of micro RNA binding sites in the single gene circuit and by using synthetic introns that are spliced out with differing efficiency also allows fine-tuning of the circuit.

The construct(s) may be adapted to express the transgene in a mammalian cell. Suitably the construct(s) may be adapted to be provided to a mammalian cell, suitably to a particular mammalian cell or cell type to which expression of the transgene is to be effected.

Advantageously, there can be provided a single gene circuit using an intron-derived microRNA in order to generate a relatively fixed level of expression across cells receiving different levels of vector-derived transgene.

As would be understood by those of skill in the art, features of the construct (a promoter, a synthetic miRNA expressed within an intron, a transgene, miRNA binding sites which provide for control of the expression of the transgene, a polyadenylation signal), should be provided relative to each other to allow functional expression of the transgene.

The construct may be adapted to include a modified Kozak sequence. Suitably, the modified Kozak sequence may be any Kozak sequence which includes any nucleic acid motif that functions as the protein translation initiation site. Suitably, the modified Kozak sequence may be any modified sequence which promotes an increase in translation initiation. Suitably, the Kozak sequence may be GCCACCATGG (SEQ ID NO: 73).

In embodiments a construct comprises (5′ to 3′):
a promoter;
at least one non-mammalian or synthetic miRNA expressed within an intron, wherein the synthetic miRNA is a sequence which is not naturally occurring;
a transgene;
one or more synthetic or non-mammalian miRNA binding site(s) which provide for control of the expression of the transgene within the transgene, wherein the synthetic miRNA binding site(s) does not naturally occur; and
a polyadenylation signal.

In embodiments a construct comprises (5′ to 3′):
a promoter;
at least one non-mammalian or synthetic miRNA expressed within an intron,
a transgene, wherein the synthetic miRNA is a sequence which is not naturally occurring;
one or more synthetic or non-mammalian miRNA binding site(s) which provide for control of the expression of the transgene within the 3′UTR, wherein the synthetic miRNA binding site(s) does not naturally occur; and
a polyadenylation signal.

In embodiments a construct comprises (5′ to 3′):
a promoter;
at least one non-mammalian or synthetic miRNA expressed within an intron wherein the synthetic miRNA is a sequence which is not naturally occurring;
a modified Kozak sequence capable of enhancing transcription of a transgene;
a transgene;
one or more synthetic or non-mammalian miRNA binding site(s) which provide for control of the expression of the transgene within the transgene or 3′ UTR, wherein the synthetic miRNA binding site(s) does not naturally occur; and
a polyadenylation signal.

In embodiments a construct comprises (5′ to 3′):
a promoter;
at least one non-mammalian or synthetic miRNA expressed within an intron, wherein the synthetic miRNA is a sequence which is not naturally occurring;
a transgene;
one or more synthetic or non-mammalian miRNA binding sites which provide for control of the expression of the transgene within the transgene or 3′UTR, wherein the synthetic miRNA binding site(s) does not naturally occur, wherein the one or more synthetic or non-mammalian miRNA binding site(s) is designed to partially ameliorate miRNA binding;
a polyadenylation signal.

In embodiments a construct comprises (5′ to 3′)
a promoter;
at least one non-mammalian or synthetic miRNA expressed within an intron, wherein the synthetic miRNA is a sequence which is not naturally occurring;
a transgene;
one or more miRNA binding site(s) which provide for control of the expression of the transgene within the transgene or 3′UTR, wherein the synthetic miRNA binding site(s) does not naturally occur;
a stability element in the 3′ UTR; and
a polyadenylation signal.

In some embodiments a construct may include a promoter, at least one non-mammalian or synthetic miRNA expressed within an intron, a transgene, one or more binding sites which provide for control of the expression of the transgene within the transgene or 3′UTR, a polyadenylation signal and, optionally, any one or more features as recited in the above embodiments. In some embodiments may comprise the one or more features recited above in the order that such features are recited.

Suitably, the constructs may be modified to provide enhanced expression, regulation and stability. Suitably the constructs may contain a reporter transgene. Suitably the constructs may contain a Kozak sequence which promotes strong expression. Suitably the constructs may contain a stability element in the 3′UTR. Suitably the constructs may contain one or more binding sites which include mutations engineered to reduce the efficacy of (but not completely ameliorate) miRNA binding.

Suitably the gene of interest may be MECP2. Alternatively, the gene of interest may be any one of the following genes of interest: FMR1, UBE3A, CDKL5, FXN, SMN1, or INS. The gene of interest may be any gene which is required to be supplied using genetic therapy for treatment of a genetic condition or developmental disorder. The gene of interest may be any gene which requires controlled expression when delivered to a subject to treat a genetic condition or developmental disorder.

### Transgene

Suitably, the transgene is a protein-coding gene which is artificially introduced into a target cell. It is provided as part of the construct of the first aspect of the invention, for example as part of a gene therapy cassette, under the control of a selected promoter. A DNA sequence of a transgene can represent a specific isoform of a specific gene. Transgene DNA sequences may be codon optimised. Codon optimisation can provide a specific and unique DNA sequence but the DNA and subsequent mRNA changes do not affect the coding sequence of the protein; i.e. the wild-type amino acid sequence is maintained.

Suitably a transgene may be selected from

>Human MECP2 -e1 isoform
(SEQ ID NO: 1)
atggccgccgccgccgccgccgcgccgagcggaggaggaggaggaggcgaggaggagagactggaagaaa
agtcagaagaccaggacctccagggcctcaaggacaaacccctcaagtttaaaaaggtgaagaaagataagaa
agaagagaaagagggcaagcatgagcccgtgcagccatcagcccaccactctgctgagcccgcagaggcagg
caaagcagagacatcagaagggtcaggctccgccccggctgtgccggaagcttctgcctcccccaaacagcggc
gctccatcatccgtgaccggggacccatgtatgatgaccccaccctgcctgaaggctggacacggaagcttaagca
aaggaaatctggccgctctgctgggaagtatgatgtgtatttgatcaatccccagggaaaagcctttcgctctaaagtg
gagttgattgcgtacttcgaaaaggtaggcgacacatccctggaccctaatgattttgacttcacggtaactgggagag
ggagcccctcccggcgagagcagaaaccacctaagaagcccaaatctcccaaagctccaggaactggcagagg
ccggggacgccccaaagggagcggcaccacgagacccaaggcggccacgtcagagggtgtgcaggtgaaaa
gggtcctggagaaaagtcctgggaagctccttgtcaagatgccttttcaaacttcgccagggggcaaggctgagggg
ggtggggccaccacatccacccaggtcatggtgatcaaacgccccggcaggaagcgaaaagctgaggccgacc
ctcaggccattcccaagaaacggggccgaaagccggggagtgtggtggcagccgctgccgccgaggccaaaaa
gaaagccgtgaaggagtcttctatccgatctgtgcaggagaccgtactccccatcaagaagcgcaagacccggga
aacggtcagcatcgaggtcaaggaagtggtgaagcccctgctggtgtccaccctcggtgagaagagcgggaaag
gactgaagacctgtaagagccctgggcggaaaagcaaggagagcagccccaaggggcgcagcagcagcgcct
cctcaccccccaagaaggagcaccaccaccatcaccaccactcagagtccccaaaggcccccgtgccactgctc
ccacccctgcccccacctccacctgagcccgagagctccgaggaccccaccagcccccctgagccccaggacttg
agcagcagcgtctgcaaagaggagaagatgcccagaggaggctcactggagagcgacggctgccccaaggag
ccagctaagactcagcccgcggttgccaccgccgccacggccgcagaaaagtacaaacaccgaggggaggga
gagcgcaaagacattgtttcatcctccatgccaaggccaaacagagaggagcctgtggacagccggacgcccgtg
accgagagagttagc
Human UBE3A
(SEQ ID NO: 2)
atgaagcgagcagctgcaaagcatctaatagaacgctactaccaccagttaactgagggctgtggaaatgaagcct
gcacgaatgagttttgtgcttcctgtccaacttttcttcgtatggataataatgcagcagctattaaagccctcgagctttat
aagattaatgcaaaactctgtgatcctcatccctccaagaaaggagcaagctcagcttaccttgagaactcgaaaggt
gcccccaacaactcctgctctgagataaaaatgaacaagaaaggcgctagaattgattttaaagatgtgacttactta
acagaagagaaggtatatgaaattcttgaattatgtagagaaagagaggattattcccctttaatccgtgttattggaag
agttttttctagtgctgaggcattggtacagagcttccggaaagttaaacaacacaccaaggaagaactgaaatctctt
caagcaaaagatgaagacaaagatgaagatgaaaaggaaaaagctgcatgttctgctgctgctatggaagaaga
ctcagaagcatcttcctcaaggataggtgatagctcacagggagacaacaatttgcaaaaattaggccctgatgatgt
gtctgtggatattgatgccattagaagggtctacaccagattgctctctaatgaaaaaattgaaactgcctttctcaatgc
acttgtatatttgtcacctaacgtggaatgtgacttgacgtatcacaatgtatactctcgagatcctaattatctgaatttgttc
attatcgtaatggagaatagaaatctccacagtcctgaatatctggaaatggctttgccattattttgcaaagcgatgagc
aagctaccccttgcagcccaaggaaaactgatcagactgtggtctaaatacaatgcagaccagattcggagaatga
tggagacatttcagcaacttattacttataaagtcataagcaatgaatttaacagtcgaaatctagtgaatgatgatgatg
ccattgttgctgcttcgaagtgcttgaaaatggtttactatgcaaatgtagtgggaggggaagtggacacaaatcacaa
tgaagaagatgatgaagagcccatccctgagtccagcgagctgacacttcaggaacttttgggagaagaaagaag
aaacaagaaaggtcctcgagtggaccccctggaaactgaacttggtgttaaaaccctggattgtcgaaaaccactta
tcccttttgaagagtttattaatgaaccactgaatgaggttctagaaatggataaagattatacttttttcaaagtagaaac
agagaacaaattctcttttatgacatgtccctttatattgaatgctgtcacaaagaatttgggattatattatgacaatagaa
ttcgcatgtacagtgaacgaagaatcactgttctctacagcttagttcaaggacagcagttgaatccatatttgagactc
aaagttagacgtgaccatatcatagatgatgcacttgtccggctagagatgatcgctatggaaaatcctgcagacttga
agaagcagttgtatgtggaatttgaaggagaacaaggagttgatgagggaggtgtttccaaagaattttttcagctggtt
gtggaggaaatcttcaatccagatattggtatgttcacatacgatgaatctacaaaattgttttggtttaatccatcttcttttg
aaactgagggtcagtttactctgattggcatagtactgggtctggctatttacaataactgtatactggatgtacattttccc
atggttgtctacaggaagctaatggggaaaaaaggaacttttcgtgacttgggagactctcacccagttctatatcaga
gtttaaaagatttattggagtatgaagggaatgtggaagatgacatgatgatcactttccagatatcacagacagatcttt
ttggtaacccaatgatgtatgatctaaaggaaaatggtgataaaattccaattacaaatgaaaacaggaaggaatttg
tcaatctttattctgactacattctcaataaatcagtagaaaaacagttcaaggcttttcggagaggttttcatatggtgacc
aatgaatctcccttaaagtacttattcagaccagaagaaattgaattgcttatatgtggaagccggaatctagatttccaa
gcactagaagaaactacagaatatgacggtggctataccagggactctgttctgattagggagttctgggaaatcgttc
attcatttacagatgaacagaaaagactcttcttgcagtttacaacgggcacagacagagcacctgtgggaggacta
ggaaaattaaagatgattatagccaaaaatggcccagacacagaaaggttacctacatctcatacttgctttaatgtgc
ttttacttccggaatactcaagcaaagaaaaacttaaagagagattgttgaaggccatcacgtatgccaaaggatttg
gcatgctg
>Human FMR1 - isoform 7
(SEQ ID NO: 3)
atggaggagctggtggtggaagtgcggggctccaatggcgctttctacaaggcatttgtaaaggatgttcatgaagatt
caataacagttgcatttgaaaacaactggcagcctgataggcagattccatttcatgatgtcagattcccacctcctgta
ggttataataaagatataaatgaaagtgatgaagttgaggtgtattccagagcaaatgaaaaagagccttgctgttggt
ggttagctaaagtgaggatgataaagggtgagttttatgtgatagaatatgcagcatgtgatgcaacttacaatgaaatt
gtcacaattgaacgtctaagatctgttaatcccaacaaacctgccacaaaagatactttccataagatcaagctggatg
tgccagaagacttacggcaaatgtgtgccaaagaggcggcacataaggattttaaaaaggcagttggtgccttttctgt
aacttatgatccagaaaattatcagcttgtcattttgtccatcaatgaagtcacctcaaagcgagcacatatgctgattga
catgcactttcggagtctgcgcactaagttgtctctgataatgagaaatgaagaagctagtaagcagctggagagttca
aggcagcttgcctcgagatttcatgaacagtttatcgtaagagaagatctgatgggtctagctattggtactcatggtgct
aatattcagcaagctagaaaagtacctggggtcactgctattgatctagatgaagatacctgcacatttcatatttatgga
gaggatcaggatgcagtgaaaaaagctagaagttttctcgaatttgctgaagatgtaatacaagttccaaggaactta
gtaggcaaagtaataggaaaaaatggaaagctgattcaggagattgtggacaagtcaggagttgtgagggtgagg
attgaggctgaaaatgagaaaaatgttccacaagaagaggaaattatgccaccaaattcccttccttccaataattca
agggttggacctaatgccccagaagaaaaaaaacatttagatataaaggaaaacagcacccatttttctcaacctaa
cagtacaaaagtccagaggggtatggtaccatttgtttttgtgggaacaaaggacagcatcgctaatgccactgttctttt
ggattatcacctgaactatttaaaggaagtagaccagttgcgtttggagagattacaaattgatgagcagttgcgacag
attggagctagttctagaccaccaccaaatcgtacagataaggaaaaaagctatgtgactgatgatggtcaaggaat
gggtcgaggtagtagaccttacagaaatagggggcacggcagacgcggtcctggatatacttcaggaactaattctg
aagcatcaaatgcttctgaaacagaatctgaccacagagatgaactcagtgattggtcattagctccaacagaggaa
gagagggagagcttcctgcgcagaggagatggacggcggcgtggagggggaggaagaggacaaggaggaag
aggacgtggaggaggcttcaaaggaaacgacgatcactcccgaacagataatcgtccacgtaatccaagagagg
ctaaaggaagaacaacagatggatcgcttcagatcagagttgactgcaataatgaaaggagtgtccacactaaaac
attacagaatacctccagtgaaggtagtcggctgcgcacgggtaaagatcgtaaccagaagaaagagaagccag
acagcgtggatggtcagcaaccactcgtgaatggagtaccc
>Human SYNGAP1
(SEQ ID NO: 4)
atgagcaggagccgagccagcatacatagagggagcatcccagctatgagttacgcaccatttcgggatgtccgcg
ggcccagtatgcaccgaactcaatacgtgcactccccatatgaccgaccaggatggaaccctaggttttgtatcatatc
tggcaaccaactgctcatgctcgacgaagatgagatccacccactcttgataagggaccgaagatccgaatctagc
agaaacaagctcttgcgaaggaccgtcagtgttccagtggaaggacggccccatggagaacacgagtaccatttg
ggtcggagcagaaggaaaagcgtgccaggaggtaagcaatacagtatggaaggtgccccagccgcaccatttag
gcccagtcagggtttcttgagtcggcgccttaagtccagcataaaacggacaaagtcccagcccaaactcgatcgca
ccagtagcttccgccagatactcccacgatttcgctccgcagatcacgatagggctaggttgatgcaatccttcaaaga
atctcactcacatgagtcactgcttagcccctccagcgcagcagaagctctggagcttaacctcgatgaggattctata
atcaagcccgttcattcaagcatcctgggtcaagagttctgtttcgaagttactacaagcagtgggactaagtgtttcgcc
tgcaggtcagccgccgagcgcgataagtggatcgaaaaccttcagcgggccgttaaaccaaacaaggacaattct
aggagggtggataacgtacttaaattgtggataatcgaagctcgcgaactccctcccaagaagagatactactgcga
actttgtctcgacgacatgctgtatgcccgaacaactagtaaaccccgcagtgcctctggagacaccgtgttttggggc
gagcacttcgagttcaataacttgcctgccgtcagggctctgagacttcacctttacagggacagtgacaagaagcga
aaaaaagacaaagcagggtatgtgggtcttgtcaccgtaccagttgccacactcgctggacgccacttcaccgaac
agtggtaccccgtcacccttcccaccggttccggcggctccggtgggatgggatccgggggaggtggagggtccgg
aggtggtagcggaggaaaaggcaagggaggttgccccgctgttcggctcaaagcaaggtatcagactatgagtatt
ctgccaatggagctctacaaagagttcgctgagtacgtaacaaatcactatagaatgctctgtgcagtactggaacct
gctctcaatgtaaaaggcaaggaagaggtagctagcgcactcgttcacattctgcagtcaactggaaaagcaaagg
attttctcagtgacatggctatgagcgaagtggatagattcatggagagagagcatttgatattccgcgaaaacacatt
ggcaaccaaagccatagaagaatacatgagactgatagggcaaaaatatctcaaggatgccataggagaatttat
acgcgccctctatgaaagtgaggaaaattgtgaggttgatcccataaagtgcacagcatcatctctggcagagcacc
aggccaatctgcgaatgtgctgtgagctggcactctgcaaggtcgtaaacagccactgtgtctttcctcgcgaactgaa
ggaagtttttgcttcctggcgcttgcggtgcgctgaacggggtcgcgaggacatagccgaccgactcatctctgccagt
ttgtttttgaggttcctctgtcctgccatcatgtctccctccctctttggcctcatgcaggagtatcccgacgaacaaacttca
agaacattgaccctcattgctaaagtgatccagaaccttgctaatttttctaaattcacttcaaaggaggatttcttgggatt
tatgaacgaattcttggaactggaatgggggagcatgcaacaatttctttacgagattagcaaccttgatactttgacta
acagcagcagtttcgaaggctatattgatttgggccgggagctctcaacccttcatgccctcctctgggaagttcttcctc
agctttccaaggaagcacttcttaagttgggtcccctcccacgccttttgaacgacatatctactgcccttcgaaatccca
atattcaacgccagccttctcgacagtccgaacgcccccgcccccagcccgtcgtcctcagagggcccagtgccga
aatgcaaggatatatgatgcgagacctgaactcttcaatagaccttcagtcttttatggctcgcggtctgaatagttctatg
gatatggccagacttccttcccctactaaagagaaacctccacctccccctccaggagggggtaaggacctgttctat
gtatcaagacccccactggcccgctcctcacctgcatattgtacatccagctccgacataactgaacccgagcaaaa
aatgcttagtgtgaacaaaagcgtcagtatgcttgaccttcagggtgacggacctggaggaaggcttaacagttccag
tgtatccaatctggctgcagtaggcgatctgctgcacagtagccaagcctcccttaccgcagctcttggtctcaggccc
gcacccgctggacgcctgtcacagggctcagggtccagcatcaccgcagctggtatgaggctctcccaaatggggg
tcaccacagacggcgtccctgcacagcaactccgcattcctctttccttccaaaacccactttttcacatggcagctgac
ggtcctggtcccccaggaggtcacggtgggggcggcggacacgggccaccctcaagccaccaccatcatcacca
ccatcaccatcacagggggggagaacctcctggggacaccttcgctccctttcacggttactcaaaatctgaggatttg
tcaagtggagttcccaagccacctgctgcaagcatcttgcatagtcacagctattcagatgagttcgggccctctggaa
ccgactttactcgcaggcagttgtcacttcaggataatttgcagcatatgctctctccaccccaaatcacaattgggccc
cagaggcccgcaccaagcggccctggaggtgggtccggtgggggcagcggtggcggaggcggaggacaacct
cctccacttcaaagaggtaagtcccagcaactcacagtcagtgctgctcaaaagccaagacccagctctggcaacc
ttctccagagcccagagcccagttacgggcctgccagaccacggcaacagagcctgtctaaagaaggcagtatag
gcggttctggggggagcggaggtgggggagggggtggcctcaaaccaagtatcaccaagcagcatagtcagaca
cccagcacattgaatcctaccatgcctgcttccgagagaacagttgcttgggtctctaatatgccacatctcagtgcaga
tatcgagagtgctcacatcgagagggaggaatacaaactgaaagagtactcaaagtctatggatgaaagtcgcctc
gacagggtcaaggagtacgaagaggaaatacactctctgaaggaacgactgcacatgtccaatcggaagttggaa
gaatatgagagaagattgttgagccaagaggaacaaacttcaaaaattttgatgcaataccaagcaaggttggaac
agagcgaaaagcggttgcgacaacagcaggccgaaaaagactcccagattaagtcaatcatcggacgccttatgc
tggtagaagaagagctgcgccgggaccatcccgcaatggctgagccacttcccgagccaaaaaaaagactcttgg
acgctcagcgggggtcattccctccctgggttcagcagaccagggtg

Suitably a functional variant of these transgenes may be provided wherein the functional variant retains the function provided by the transgene and has at least 60% sequence identity, at least 70% sequence identity, at least 80% sequence identity, at least 90% sequence identity, at least 95% sequence identity, at least 97% sequence identity, at least 99% sequence identity. Suitably a functional variant may be a fragment of the transgene which provides the function of the transgene. Suitably where the miRNA binding sites, which provide for control of the expression of the transgene, are provided within the transgene, the miRNA binding sites are provided in the functional variant such that the miRNA can bind and control the expression of the transgene.

Sequence identity can be determined by any methods known in the art. Suitably sequence identity may be determined over the full length of the transgene.

Suitable transgenes include those based on any single gene disorders for which controlled expression of the transgene is desired. Suitable transgenes include those based on any monogenic disorder for which controlled expression of the transgene is desired. Additional exemplary transgenes include those based on single gene CNS disorders for which controlled expression of the transgene is desired. The nervous system expresses many genes that are known to be deleterious to nervous system function when overexpressed. However, the present invention is applicable to any situation in where transgene overexpression is deleterious including gene therapy for non-CNS disorders. An example would include dystrophin gene replacement in muscle cells whereby moderate overexpression does not cause deleterious adverse effects but when very high levels of overexpression leads to severe cardiac toxicity.

miRNAs Expression from within an Intron

Micro RNAs (miRNAs) are a class of small, single-stranded, non-coding RNAs of ˜22 nucleotides in length. Most miRNAs are transcribed by RNA polymerase II, either as independent transcripts or as RNAs embedded within introns of mRNAs. Primary miRNA transcripts are processed into ˜70 nt hairpin precursor miRNAs and then finally to ˜22 nt mature miRNAs by two RNase III enzymes (Drosha and Dicer). miRNAs function by regulating protein levels, targeting messenger RNAs (mRNAs) for translational repression and/or mRNA degradation.

The inventors have developed non-mammalian or synthetic miRNAs of the invention that are capable of knocking-down expression of transcripts containing the respective binding region. In some instances of the invention these are insect-derived miRNA sequences originally designed to target the firefly luciferase protein. In other instances, they are synthetic miRNA sequences, with no orthology to naturally occurring miRNAs. In some instances, synthetic miRNA sequences are designed to target codon optimised coding sequences, where the coding sequence is altered at the DNA level while retaining the same amino acid sequence. In a gene therapy context, this allows exogenously delivered transgenes to be exclusively targeted by the synthetic miRNAs, whilst endogenous genes are unaffected. In a final instance of the invention, completely novel synthetic miRNA sequences were created by in silico generation of large DNA sequences which were used with existing miRNA design tools to identify sequences suitable for miRNA targeting. Suitably, since all of these miRNAs are non-mammalian or synthetic, they have no predicted endogenous targets within the mammalian transcriptome.

Suitably a miRNA may be embedded within different introns. Examples of such introns are provided below. The human EF1a intron is the intron present in the commonly used EF1a promoter and is known to splice efficiently. The MINIX intron is also known to splice efficiently and is useful in a gene therapy context for its short sequence. The inventors have shown that that the EF1a promoter and MINIX intron can work in combination. The inventors have also shown that the JeT promoter and MINIX intron work in combination.

Suitably an intron may be selected from:

>human_EF1a_intron_A
(SEQ ID NO: 5)
gtaagtgccgtgtgtggttcccgcgggcctggcctctttacgggttatggc
ccttgcgtgccttgaattacttccacgcccctggctgcagtacgtgattct
tgatcccgagcttcgggttggaagtgggtgggagagttcgaggccttgcgc
ttaaggagccccttcgcctcgtgcttgagttgaggcctggcttgggcgctg
gggccgccgcgtgcgaatctggtggcaccttcgcgcctgtctcgctgcttt
cgataagtctctagccatttaaaatttttgatgacctgctgcgacgctttt
tttctggcaagatagtcttgtaaatgcgggccaagatctgcacactggtat
ttcggtttttggggccgcgggggcgacggggcccgtgcgtcccagcgcaca
tgttcggcgaggcggggcctgcgagcgcggccaccgagaatcggacggggg
tagtctcaagctggccggcctgctctggtgcctggcctcgcgccgccgtgt
atcgccccgccctgggcggcaaggctggcccggtcggcaccagttgcgtga
gcggaaagatggccgcttcccggccctgctgcagggagctcaaaatggagg
acgcggcgctcgggagagcgggcgggtgagtcacccacacaaaggaaaagg
gcctttccgtcctcagccgtcgcttcatgtgactccacggagtaccgggcg
ccgtccaggcacctcgattagttctcgagcttttggagtacgtcgtcttta
ggttggggggaggggttttatgcgatggagtttccccacactgagtgggtg
gagactgaagttaggccagcttggcacttgatgtaattctccttggaattt
gccctttttgagtttggatcttggttcattctcaagcctcagacagtggtt
caaagtttttttcttccatttcag
>MINIX_artificial_intron
(SEQ ID NO: 6)
gtaagagcctagcatgtagaactggttacctgcagcccaagcttgctgcac
gtctagggctcaccgggtttccttgatgaggtaccgacatacttatcctgt
cccttttttttccacag

Suitably a miRNA may be provided by a non-mammalian miRNA originally targeted against firefly lucifersase (ffluc1).

Non-Mammalian miRNA: (Luciferase)

> ffluc1_full_miRNA_sequence
(SEQ ID NO: 7)
5′ -AACGATATGGGCTGAATACAA-3′
>ffluc1_seed_sequence
5′ -ACGATA-3′

A BLAST search determined that there are no identical (21 bp) matches to this RNA in any RNA transcripts produced in human cells (thus, it is a “non-mammalian” sequence). Studies have shown that miRNAs can tolerate mismatches in target sites if there is exact complementarity to the seed sequence. The seed sequence is usually situated at positions 2-7 in the 5′ region of the miRNA and is essential from miRNA binding. However, no potential off-target RNAs contained an exact seed sequence match.

miRNAs are embedded in a hairpin loop structure to allow correct recognition and processing. Suitably an embedded non-mammalian miRNA may be selected from

>ffluc1
(SEQ ID NO: 9)
tgtttgaatgaggcttcagtactttacagaatcgttgcctgcacatcttgg
aaacacttgctgggattacttcgacttcttaacccaacagaaggctcgaga
aggtatattgctgttgacagtgagcgaaacgatatgggctgaatacaatag
tgaagccacagatgtattgtattcagcccatatcgttgtgcctactgcctc
ggacttcaaggggctagaattcgagcaattatcttgtttactaaaactgaa
taccttgctatctctttgatacatttttacaaagctgaattaaaatggtat
aaattaaatcacttt
>ffluc9
(SEQ ID NO: 10)
tgtttgaatgaggcttcagtactttacagaatcgttgcctgcacatcttgg
aaacacttgctgggattacttcgacttcttaacccaacagaaggctcgaga
aggtatattgctgttgacagtgagcgaaacgtgaattgctcaacagtatag
tgaagccacagatgtatactgttgagcaattcacgttgtgcctactgcctc
ggacttcaaggggctagaattcgagcaattatcttgtttactaaaactgaa
taccttgctatctctttgatacatttttacaaagctgaattaaaatggtat
aaattaaatcacttt
>ffluc18
(SEQ ID NO: 11)
tgtttgaatgaggcttcagtactttacagaatcgttgcctgcacatcttgg
aaacacttgctgggattacttcgacttcttaacccaacagaaggctcgaga
aggtatattgctgttgacagtgagcgacccgacgatgacgccggtgaatag
tgaagccacagatgtattcaccggcgtcatcgtcggggtgcctactgcctc
ggacttcaaggggctagaattcgagcaattatcttgtttactaaaactgaa
taccttgctatctctttgatacatttttacaaagctgaattaaaatggtat
aaattaaatcacttt
>ffluc22
(SEQ ID NO: 12)
tgtttgaatgaggcttcagtactttacagaatcgttgcctgcacatcttgg
aaacacttgctgggattacttcgacttcttaacccaacagaaggctcgaga
aggtatattgctgttgacagtgagcgattcgatagggacaagacaatttag
tgaagccacagatgtaaattgtcttgtccctatcgaagtgcctactgcctc
ggacttcaaggggctagaattcgagcaattatcttgtttactaaaactgaa
taccttgctatctctttgatacatttttacaaagctgaattaaaatggtat
aaattaaatcacttt

Suitably a miRNA may be provided by a novel synthetic miRNA originally targeted against randomly generated sequence, with no orthology to mammalian, insect or plant miRNAs.

Suitably an embedded synthetic miRNA may be selected from:

>novel_seq_1
(SEQ ID NO: 13)
tgtttgaatgaggcttcagtactttacagaatcgttgcctgcacatcttgg
aaacacttgctgggattacttcgacttcttaacccaacagaaggctcgaga
aggtatattgctgttgacagtgagcgagcatgttacgggacttcttattag
tgaagccacagatgtaataagaagtcccgtaacatgcgtgcctactgcctc
ggacttcaaggggctagaattcgagcaattatcttgtttactaaaactgaa
taccttgctatctctttgatacatttttacaaagctgaattaaaatggtat
aaattaaatcacttt
>novel_seq_2
(SEQ ID NO: 14)
tgtttgaatgaggcttcagtactttacagaatcgttgcctgcacatcttgg
aaacacttgctgggattacttcgacttcttaacccaacagaaggctcgaga
aggtatattgctgttgacagtgagcgagtgaggagcagcggatcttaatag
tgaagccacagatgtattaagatccgctgctcctcacgtgcctactgcctc
ggacttcaaggggctagaattcgagcaattatcttgtttactaaaactgaa
taccttgctatctctttgatacatttttacaaagctgaattaaaatggtat
aaattaaatcacttt
>novel_seq_3
(SEQ ID NO: 15)
tgtttgaatgaggcttcagtactttacagaatcgttgcctgcacatcttgg
aaacacttgctgggattacttcgacttcttaacccaacagaaggctcgaga
aggtatattgctgttgacagtgagcgagtcatgtcgtcacggaacttatag
tgaagccacagatgtataagttccgtgacgacatgacgtgcctactgcctc
ggacttcaaggggctagaattcgagcaattatcttgtttactaaaactgaa
taccttgctatctctttgatacatttttacaaagctgaattaaaatggtat
aaattaaatcacttt
>novel_seq_4
(SEQ ID NO: 16)
tgtttgaatgaggcttcagtactttacagaatcgttgcctgcacatcttgg
aaacacttgctgggattacttcgacttcttaacccaacagaaggctcgaga
aggtatattgctgttgacagtgagcgagagaagtgcggatttcgtatttag
tgaagccacagatgtaaatacgaaatccgcacttctcgtgcctactgcctc
ggacttcaaggggctagaattcgagcaattatcttgtttactaaaactgaa
taccttgctatctctttgatacatttttacaaagctgaattaaaatggtat
aaattaaatcacttt
>novel_seq_5 (ran1g)
(SEQ ID NO: 17)
tgtttgaatgaggcttcagtactttacagaatcgttgcctgcacatcttgg
aaacacttgctgggattacttcgacttcttaacccaacagaaggctcgaga
aggtatattgctgttgacagtgagcgaaagcgccaaaggagtctgtgatag
tgaagccacagatgtatcacagactcctttggcgcttgtgcctactgcctc
ggacttcaaggggctagaattcgagcaattatcttgtttactaaaactgaa
taccttgctatctctttgatacatttttacaaagctgaattaaaatggtat
aaattaaatcacttt
>novel_seq_6 (ran2g)
(SEQ ID NO: 18)
tgtttgaatgaggcttcagtactttacagaatcgttgcctgcacatcttgg
aaacacttgctgggattacttcgacttcttaacccaacagaaggctcgaga
aggtatattgctgttgacagtgagcgaaagtgcggatttcgtatttgctag
tgaagccacagatgtagcaaatacgaaatccgcacttgtgcctactgcctc
ggacttcaaggggctagaattcgagcaattatcttgtttactaaaactgaa
taccttgctatctctttgatacatttttacaaagctgaattaaaatggtat
aaattaaatcacttt
>novel_seq_7
(SEQ ID NO: 19)
tgtttgaatgaggcttcagtactttacagaatcgttgcctgcacatcttgg
aaacacttgctgggattacttcgacttcttaacccaacagaaggctcgaga
aggtatattgctgttgacagtgagcgaaagtggatgcgatgcgattgctag
tgaagccacagatgtagcaatcgcatcgcatccacttgtgcctactgcctc
ggacttcaaggggctagaattcgagcaattatcttgtttactaaaactgaa
taccttgctatctctttgatacatttttacaaagctgaattaaaatggtat
aaattaaatcacttt
>novel_seq_8
(SEQ ID NO: 20)
tgtttgaatgaggcttcagtactttacagaatcgttgcctgcacatcttgg
aaacacttgctgggattacttcgacttcttaacccaacagaaggctcgaga
aggtatattgctgttgacagtgagcgaaacggtatccgcaacttgcgatag
tgaagccacagatgtatcgcaagttgcggataccgttgtgcctactgcctc
ggacttcaaggggctagaattcgagcaattatcttgtttactaaaactgaa
taccttgctatctctttgatacatttttacaaagctgaattaaaatggtat
aaattaaatcacttt

Suitably an embedded synthetic miRNA may be targeted against the coding sequence of a target gene (i.e. a therapeutic transgene).

Target genes may be codon optimized and synthetic miRNAs, with no orthology to mammalian, insect or plant miRNAs, screened for ability to target the codon optimized transgene without targeting endogenous transcripts of the same gene. Suitably an embedded synthetic miRNA targeting a coding optimised sequence may be selected from:

>MECP2_coding_1
(SEQ ID NO: 21)
tgtttgaatgaggcttcagtactttacagaatcgttgcctgcacatcttgg
aaacacttgctgggattacttcgacttcttaacccaacagaaggctcgaga
aggtatattgctgttgacagtgagcgaaagtacgatgtttacttgatctag
tgaagccacagatgtagatcaagtaaacatcgtacttgtgcctactgcctc
ggacttcaaggggctagaattcgagcaattatcttgtttactaaaactgaa
taccttgctatctctttgatacatttttacaaagctgaattaaaatggtat
aaattaaatcacttt
>MECP2_coding_2
(SEQ ID NO: 22)
tgtttgaatgaggcttcagtactttacagaatcgttgcctgcacatcttgg
aaacacttgctgggattacttcgacttcttaacccaacagaaggctcgaga
aggtatattgctgttgacagtgagcgaaagccgctcttggtctctacctag
tgaagccacagatgtaggtagagaccaagagcggcttgtgcctactgcctc
ggacttcaaggggctagaattcgagcaattatcttgtttactaaaactgaa
taccttgctatctctttgatacatttttacaaagctgaattaaaatggtat
aaattaaatcacttt
>MECP2_coding_3
(SEQ ID NO: 23)
tgtttgaatgaggcttcagtactttacagaatcgttgcctgcacatcttgg
aaacacttgctgggattacttcgacttcttaacccaacagaaggctcgaga
aggtatattgctgttgacagtgagcgaaagtccgaagatcaagacctgtag
tgaagccacagatgtacaggtcttgatcttcggacttgtgcctactgcctc
ggacttcaaggggctagaattcgagcaattatcttgtttactaaaactgaa
taccttgctatctctttgatacatttttacaaagctgaattaaaatggtat
aaattaaatcacttt
>MECP2_coding_4
(SEQ ID NO: 24)
tgtttgaatgaggcttcagtactttacagaatcgttgcctgcacatcttgg
aaacacttgctgggattacttcgacttcttaacccaacagaaggctcgaga
aggtatattgctgttgacagtgagcgagagtccagtatacgcagtgtatag
tgaagccacagatgtatacactgcgtatactggactcgtgcctactgcctc
ggacttcaaggggctagaattcgagcaattatcttgtttactaaaactgaa
taccttgctatctctttgatacatttttacaaagctgaattaaaatggtat
aaattaaatcacttt
> MECP2_coding_5
(SEQ ID NO: 25)
tgtttgaatgaggcttcagtactttacagaatcgttgcctgcacatcttgg
aaacacttgctgggattacttcgacttcttaacccaacagaaggctcgaga
aggtatattgctgttgacagtgagcgagtccagtatacgcagtgtacatag
tgaagccacagatgtatgtacactgcgtatactggacgtgcctactgcctc
ggacttcaaggggctagaattcgagcaattatcttgtttactaaaactgaa
taccttgctatctctttgatacatttttacaaagctgaattaaaatggtat
aaattaaatcacttt
> MECP2_coding_6
(SEQ ID NO: 26)
Tgtttgaatgaggcttcagtactttacagaatcgttgcctgcacatcttgg
aaacacttgctgggattacttcgacttcttaacccaacagaaggctcgaga
aggtatattgctgttgacagtgagcgagactcgggaaaccgttagtattag
tgaagccacagatgtaatactaacggtttcccgagtcgtgcctactgcctc
ggacttcaaggggctagaattcgagcaattatcttgtttactaaaactgaa
taccttgctatctctttgatacatttttacaaagctgaattaaaatggtat
aaattaaatcacttt
>SYNGAP1_coding_1
(SEQ ID NO: 27)
tgtttgaatgaggcttcagtactttacagaatcgttgcctgcacatcttgg
aaacacttgctgggattacttcgacttcttaacccaacagaaggctcgaga
aggtatattgctgttgacagtgagcgagcgccttaagtccagcataaatag
tgaagccacagatgtatttatgctggacttaaggcgcgtgcctactgcctc
ggacttcaaggggctagaattcgagcaattatcttgtttactaaaactgaa
taccttgctatctctttgatacatttttacaaagctgaattaaaatggtat
aaattaaatcacttt
> SYNGAP1_coding_2
(SEQ ID NO: 28)
tgtttgaatgaggcttcagtactttacagaatcgttgcctgcacatcttgg
aaacacttgctgggattacttcgacttcttaacccaacagaaggctcgaga
aggtatattgctgttgacagtgagcgagagttctgtttcgaagttacttag
tgaagccacagatgtaagtaacttcgaaacagaactcgtgcctactgcctc
ggacttcaaggggctagaattcgagcaattatcttgtttactaaaactgaa
taccttgctatctctttgatacatttttacaaagctgaattaaaatggtat
aaattaaatcacttt
> SYNGAP1_coding_3
(SEQ ID NO: 29)
tgtttgaatgaggcttcagtactttacagaatcgttgcctgcacatcttgg
aaacacttgctgggattacttcgacttcttaacccaacagaaggctcgaga
aggtatattgctgttgacagtgagcgaggcgagcacttcgagttcaattag
tgaagccacagatgtaattgaactcgaagtgctcgccgtgcctactgcctc
ggacttcaaggggctagaattcgagcaattatcttgtttactaaaactgaa
taccttgctatctctttgatacatttttacaaagctgaattaaaatggtat
aaattaaatcacttt
> SYNGAP1_coding_4
(SEQ ID NO: 30)
tgtttgaatgaggcttcagtactttacagaatcgttgcctgcacatcttgg
aaacacttgctgggattacttcgacttcttaacccaacagaaggctcgaga
aggtatattgctgttgacagtgagcgaaagatccgaatctagcagaaatag
tgaagccacagatgtatttctgctagattcggatcttgtgcctactgcctc
ggacttcaaggggctagaattcgagcaattatcttgtttactaaaactgaa
taccttgctatctctttgatacatttttacaaagctgaattaaaatggtat
aaattaaatcacttt
> SYNGAP1_coding_5
(SEQ ID NO: 31)
tgtttgaatgaggcttcagtactttacagaatcgttgcctgcacatcttgg
aaacacttgctgggattacttcgacttcttaacccaacagaaggctcgaga
aggtatattgctgttgacagtgagcgaaaggtcgtaaacagccactgttag
tgaagccacagatgtaacagtggctgtttacgaccttgtgcctactgcctc
ggacttcaaggggctagaattcgagcaattatcttgtttactaaaactgaa
taccttgctatctctttgatacatttttacaaagctgaattaaaatggtat
aaattaaatcacttt
> SYNGAP1_coding_6
(SEQ ID NO: 32)
tgtttgaatgaggcttcagtactttacagaatcgttgcctgcacatcttgg
aaacacttgctgggattacttcgacttcttaacccaacagaaggctcgaga
aggtatattgctgttgacagtgagcgaaagaggaaatacactctctgatag
tgaagccacagatgtatcagagagtgtatttcctcttgtgcctactgcctc
ggacttcaaggggctagaattcgagcaattatcttgtttactaaaactgaa
taccttgctatctctttgatacatttttacaaagctgaattaaaatggtat
aaattaaatcacttt

miRNAs work by binding to specific sequences complementary to the mature miRNA sequence. These binding sites may be located in the 3′ untranslated region (3′UTR) of endogenous mRNAs. The binding sites may alternatively be located in the 5′UTR, exons, and introns. In further alternative embodiments a binding site may be located within a codon optimised transgene sequence. Suitably the miRNA binding sites which provide for control of the expression of the transgene may be provided within the 3′ UTR, the 5′ UTR or within the transgene.

Suitably, a ‘seed’ sequence in the binding site forms Watson-Crick pairs with bases at the 5′ end of the miRNA, at positions 2 through 7/8. However, the skilled person would understand the way in which binding specificity and strength, for example based on sequence conservation, strong base-pairing at the 3′ end of the miRNA, local AU content and location of miRNA binding sites within the 3′ UTR may be altered.

Suitably, different numbers of binding sites can be used to alter the strength of transgene control. In addition, mismatches introduced into the binding site can be used to lower the level of transgene control. Such changes enable setting the level of dosage insensitivity.

Suitably, the binding sites may be mutated to reduce, but not completely inhibit, miRNA-target binding. Suitably, these mutations may be used to enhance expression of the transgene, whilst still maintaining regulatory control of transgene expression, by having some target miRNA still bind to binding sites.

Successful miRNA-target binding usually results in a knock-down of protein levels, either via translational repression or mRNA degradation mechanisms.

Suitably a non-mammalian or synthetic miRNA binding site may be selected from

>ffluc1_x1_binding_site
(SEQ ID NO: 33)
gctatgaaacgatatgggctgaatacaaatcacag
>ffluc1_x3_binding_sites
(SEQ ID NO: 34)
gctatgaaacgatatgggctgaatacaaatcacaggctatgaaacgatatgggctgaatacaaatcacaggctatg
aaacgatatgggctgaatacaaatcacag
>ffluc1_x6_binding_sites
(SEQ ID NO: 35)
gctatgaaacgatatgggctgaatacaaatcacaggctatgaaacgatatgggctgaatacaaatcacaggctatg
aaacgatatgggctgaatacaaatcacaggctatgaaacgatatgggctgaatacaaatcacaggctatgaaacg
atatgggctgaatacaaatcacaggctatgaaacgatatgggctgaatacaaatcacag
>ffluc1_x3_binding_sites_1bp_central_mismatch
(SEQ ID NO: 36)
gctatgaaacgatatgggcggaatacaaatcacaggctatgaaacgatatgggcggaatacaaatcacaggctat
gaaacgatatgggcggaatacaaatcacag
> ffluc1_x3_binding_sites_3bp_central_mismatch
(SEQ ID NO: 37)
gctatgaaacgatatgttatgaatacaaatcacaggctatgaaacgatatgttatgaatacaaatcacaggctatgaa
acgatatgttatgaatacaaatcacag
>ffluc1_x3_binding_sites_3′_mismatch
(SEQ ID NO: 38)
Gctatgacccaaactgtgaagaatacaaatcacaggctatgagtgtctatcacccgaatacaaatcacaggctatga
ctaggcccgtttcgaatacaaatcacag
>ffluc1_x3_binding_sites_mutant_1
(SEQ ID NO: 39)
gctatgaaacgatatgcgctgaatacaaatcacaggctatgaaacgatatgcgctgaatacaaatcacaggctatga
aacgatatgcgctgaatacaaatcacag
>ffluc1_binding_sites_mutant_2
(SEQ ID NO: 40)
gctatgaaacgatatgggctgaatacaaatcacaggctatgaaacgatatgggctgaatacaaatcacaggctatg
aaacgatatgggctgaatacaaatcacag
>ffluc1_x3_binding_sites_mutant_3
(SEQ ID NO: 41)
gctatgaaacgatatgggctcaatacaaatcacaggctatgaaacgatatgggctcaatacaaatcacaggctatga
aacgatatgggctcaatacaaatcacag
>ffluc1_x3_binding_sites_mutant_4
(SEQ ID NO: 42)
gctatgaaacgatatgggctgattacaaatcacaggctatgaaacgatatgggctgattacaaatcacaggctatga
aacgatatgggctgattacaaatcacag
>ffluc1_x3_binding_sites_mutant_5
(SEQ ID NO: 43)
gctatgaaacgatatgggctgaattcaaatcacaggctatgaaacgatatgggctgaattcaaatcacaggctatga
aacgatatgggctgaattcaaatcacag
>ffluc1_x3_binding_sites_mutant_6
(SEQ ID NO: 44)
gctatgaaacgatatgggctgaatactaatcacaggctatgaaacgatatgggctgaatactaatcacaggctatga
aacgatatgggctgaatactaatcacag
>ffluc9_x3_binding_sites
(SEQ ID NO: 45)
gctatgaaacgtgaattgctcaacagtaatcacaggctatgaaacgtgaattgctcaacagtaatcacaggctatgaa
acgtgaattgctcaacagtaatcacag
>ffluc18_x3_binding_sites
(SEQ ID NO: 46)
gctatgacccgacgatgacgccggtgaaatcacaggctatgacccgacgatgacgccggtgaaatcacaggctat
gacccgacgatgacgccggtgaaatcacag
>ffluc22_x3_binding_sites
(SEQ ID NO: 47)
gctatgattcgatagggacaagacaattatcacaggctatgattcgatagggacaagacaattatcacaggctatgat
tcgatagggacaagacaattatcacag
The following synthetic sequences were designed such that the coding sequence
was optimised and the miRNAs targeting them designed to target the parts of the
sequence that are different from mammalian endogenous sequence.
>Novel_seq_1_3x_binding_sites
(SEQ ID NO: 48)
gctatgagcatgttacgggacttcttatatcacaggctatgagcatgttacgggacttcttatatcacaggctatgagc
atgttacgggacttcttatatcacag
>Novel_seq_2_3x_binding_sites
(SEQ ID NO: 49)
gctatgagtgaggagcagcggatcttaaatcacaggctatgagtgaggagcagcggatcttaaatcacaggctatg
agtgaggagcagcggatcttaaatcacag
>Novel_seq_3_3x_binding_sites
(SEQ ID NO: 50)
gctatgagtcatgtcgtcacggaacttaatcacaggctatgagtcatgtcgtcacggaacttaatcacaggctatgagt
catgtcgtcacggaacttaatcacag
>Novel_seq_4_3x_binding_sites
(SEQ ID NO: 51)
gctatgagagaagtgcggatttcgtattatcacaggctatgagagaagtgcggatttcgtattatcacaggctatgaga
gaagtgcggatttcgtattatcacag
>Novel_seq_5_3x_binding_sites
(SEQ ID NO: 52)
gctatgaaagcgccaaaggagtctgtgaatcacaggctatgaaagcgccaaaggagtctgtgaatcacaggctatg
aaagcgccaaaggagtctgtgaatcacag
>Novel_seq_6_3x_binding_sites
(SEQ ID NO: 53)
gctatgaaagtgcggatttcgtatttgcatcacaggctatgaaagtgcggatttcgtatttgcatcacaggctatgaaa
gtgcggatttcgtatttgcatcacag
>Novel_seq_7_3x_binding_sites
(SEQ ID NO: 54)
gctatgaaagtggatgcgatgcgattgcatcacaggctatgaaagtggatgcgatgcgattgcatcacaggctatga
aagtggatgcgatgcgattgcatcacag
>Novel_seq_8_3x_binding_sites
(SEQ ID NO: 55)
gctatgaaacggtatccgcaacttgcgaatcacaggctatgaaacggtatccgcaacttgcgaatcacaggctatga
aacggtatccgcaacttgcgaatcacag
>MECP2_coding_1_binding_site
(SEQ ID NO: 56)
aagtacgatgtttacttgatc
> MECP2_coding_2_binding_site
(SEQ ID NO: 57)
aagccgctcttggtctctacc
> MECP2_coding_3_binding_site
(SEQ ID NO: 58)
aagtccgaagatcaagacctg
> MECP2_coding_4_binding_site
(SEQ ID NO: 59)
gagtccagtatacgcagtgta
> MECP2_coding_5_binding_site
(SEQ ID NO: 60)
gtccagtatacgcagtgtaca
> MECP2_coding_6_binding_site
(SEQ ID NO: 61)
gactcgggaaaccgttagtat
>SYNGAP1_coding_1_binding_site
(SEQ ID NO: 62)
gcgccttaagtccagcataaa
> SYNGAP1_coding_2_binding_site
(SEQ ID NO: 63)
gagttctgtttcgaagttact
> SYNGAP1_coding_3_binding_site
(SEQ ID NO: 64)
ggcgagcacttcgagttcaat
> SYNGAP1_coding_4_binding_site
(SEQ ID NO: 65)
Aagatccgaatctagcagaaa
> SYNGAP1_coding_5_binding_site
(SEQ ID NO: 66)
aaggtcgtaaacagccactgt
> SYNGAP1_coding_6_binding_site
(SEQ ID NO: 67)
aagaggaaatacactctctga

### Promoter

Any suitable promoter, constitutive or conditional, can be used to drive expression of the transgene. Suitably a promoter may comprise an Ef1a promoter, CAG promoter, Jet promoter, CMV promoter, CBA promoter, CBH promoter, Synapsin1 promoter, Mecp2 promoter, U1a promoter, U6 promoter, ubiquitin C promoter, neuron-specific enolase promoter, oligodendrocyte transcription factor 1 or GFAP promoter. In embodiments the feedforward miRNA can be incorporated into an intronic sequence coupled to suitable, for example any of the above promoters.

The exact promoter used will be dependent on the strength of expression needed and, in cases of larger genes, the amount of packaging capacity available, for example in an AAV delivery vector. Suitable promotors may be provided by:

>EF1a_promoter
(SEQ ID NO: 68)
Agtaattcatacaaaaggactcgcccctgccttggggaatcccagggaccg
tcgttaaactcccactaacgtagaacccagagatcgctgcgttcccgcccc
ctcacccgcccgctctcgtcatcactgaggtggagaagagcatgcgtgagg
ctccggtgcccgtcagtgggcagagcgcacatcgcccacagtccccgagaa
gttggggggaggggtcggcaattgaaccggtgcctagagaaggtggcgcgg
ggtaaactgggaaagtgatgtcgtgtactggctccgcctttttcccgaggg
tgggggagaaccgtatataagtgcagtagtcgccgtgaacgttctttttcg
caacgggtttgccgccagaacacag
>Jet_promoter
(SEQ ID NO: 69)
gggcggagttagggcggagccaatcagcgtgcgccgttccgaaagttgcct
tttatggctgggcggagaatgggcggtgaacgccgatgattatataaggac
gcgccgggtgtggcacagctagttccgtcgcagccgggatttgggtcgcgg
ttcttgtttgt
>CMV-CBA promoter
(SEQ ID NO: 76)
CTAGTTATTAATAGTAATCAATTACGGGGTCATTAGTTCATAGCCCATATA
TGGAGTTCCGCGTTACATAACTTACGGTAAATGGCCCGCCTGGCTGACCGC
CCAACGACCCCCGCCCATTGACGTCAATAATGACGTATGTTCCCATAGTAA
CGCCAATAGGGACTTTCCATTGACGTCAATGGGTGGAGTATTTACGGTAAA
CTGCCCACTTGGCAGTACATCAAGTGTATCATATGCCAAGTACGCCCCCTA
TTGACGTCAATGACGGTAAATGGCCCGCCTGGCATTATGCCCAGTACATGA
CCTTATGGGACTTTCCTACTTGGCAGTACATCTACGTATTAGTCATCGCTA
TTACCATGGTCGAGGTGAGCCCCACGTTCTGCTTCACTCTCCCCATCTCCC
CCCCCTCCCCACCCCCAATTTTGTATTTATTTATTTTTTAATTATTTTGTG
CAGCGATGGGGGCGGGGGGGGGGGGGGGGCGCGCGCCAGGCGGGGCGGGGC
GGGGCGAGGGGCGGGGCGGGGCGAGGCGGAGAGGTGCGGCGGCAGCCAATC
AGAGCGGCGCGCTCCGAAAGTTTCCTTTTATGGCGAGGCGGCGGCGGCGGC
GGCCCTATAAAAAGCGAAGCGCGCGGCGGGCG

### Polyadenylation Signal

The approach can be used with synthetic polyA sequences or truncated fragments of native polyA sequences. In embodiments the feed forward miRNA binding sites can be incorporated within the 3′UTR. Suitably the miRNA binding sites can be incorporated within the 3′UTR unless embedded within the transgene sequence.

Any suitable polyadenylation signal as known in the art may be utilised. Suitably, the polyA signal may be

>sv40 polyA signal
(SEQ ID NO: 70)
Aacttgtttattgcagcttataatggttacaaataaagcaatagcatcaca
aatttcacaaataaagcatttttttcactgcattctagttgtggtttgtcc
aaactcatcaatgtatctta
>BGH polyA signal
(SEQ ID NO: 71)
ctgtgccttctagttgccagccatctgttgtttgcccctcccccgtgcctt
ccttgaccctggaaggtgccactcccactgtcctttcctaataaaatgagg
aaattgcatcgcattgtctgagtaggtgtcattctattctggggggtgggg
tggggcaggacagcaagggggaggattgggaagacaatagcaggcatgctg
gggatgcggtgggctctatgg
>SpA
(SEQ ID NO: 72)
aataaagagctcagatgcatcgatcagagtgtgttggttttttgtgtg

### Stability Element

Suitably, a stability element to increase transgene expression may be included. Suitably, the stability element may be located in the 3′ UTR. Suitably the stability element may be

> WPRE
(SEQ ID NO: 74)
AATCAACCTCTGGATTACAAAATTTGTGAAAGATTGACTGGTATTCTTAAC
TATGTTGCTCCTTTTACGCTATGTGGATACGCTGCTTTAATGCCTTTGTAT
CATGCTATTGCTTCCCGTATGGCTTTCATTTTCTCCTCCTTGTATAAATCC
TGGTTGCTGTCTCTTTATGAGGAGTTGTGGCCCGTTGTCAGGCAACGTGGC
GTGGTGTGCACTGTGTTTGCTGACGCAACCCCCACTGGTTGGGGCATTGCC
ACCACCTGTCAGCTCCTTTCCGGGACTTTCGCTTTCCCCCTCCCTATTGCC
ACGGCGGAACTCATCGCCGCCTGCCTTGCCCGCTGCTGGACAGGGGCTCGG
CTGTTGGGCACTGACAATTCCGTGGTGTTGTCGGGGAAATCATCGTCCTTT
CCTTGGCTGCTCGCCTGTGTTGCCACCTGGATTCTGCGCGGGACGTCCTTC
TGCTACGTCCCTTCGGCCCTCAATCCAGCGGACCTTCCTTCCCGCGGCCTG
CTGCCGGCTCTGCGGCCTCTTCCGCGTCTTCGCCTTCGCCCTCAGACGAGT
CGGATCTCCCTTTGGGCCGCCTCCCCGC
> WPRE3
(SEQ ID NO: 75)
ATAATCAACCTCTGGATTACAAAATTTGTGAAAGATTGACTGGTATTCTTA
ACTATGTTGCTCCTTTTACGCTATGTGGATACGCTGCTTTAATGCCTTTGT
ATCATGCTATTGCTTCCCGTATGGCTTTCATTTTCTCCTCCTTGTATAAAT
CCTGGTTAGTTCTTGCCACGGCGGAACTCATCGCCGCCTGCCTTGCCCGCT
GCTGGACAGGGGCTCGGCTGTTGGGCACTGACAATTCCGTGGT.

### Vector

The miRNA feed-forward construct of the invention is designed to work in vivo. To deliver these constructs to the requisite tissues/organs, any suitable viral vector can be utilised. In an embodiment, a viral vector may be an adeno-associated virus (AAV) delivery system or other therapeutic viral vector systems including lentivirus, adenovirus, herpes simplex virus, retrovirus, alphavirus, flaviviruses, rhadboviruses, measles virus, picornaviruses and poxviruses. For AAV, the entire construct (promoter, miRNA, transgene, binding site, polyA) can be cloned into an AAV-compatible plasmid where it is flanked by inverted terminal repeat (ITR) sequences. AAV production has strict size limits, so the entire construct must be no more than 4.4 kb (excluding ITRs). This size limit can restrict the use of certain transgenes, which would take up the bulk of the available space. Alternative, smaller promoters and polyA's can be used to accommodate larger transgenes. Suitably, in constructs, the 3′UTR region could be removed, and a synthetic miRNA targeted to the codon-optimised sequence of the transgene. As the codon-optimised transgene has a different DNA/mRNA sequence, endogenous mRNA from the gene of interest (G01) would not be targeted.

According to a second aspect of the present invention there is provided a vector comprising a construct of the first aspect of the invention.

Suitably the construct may be provided in a viral vector to allow delivery of the construct to target cells. A target cell may be cells of the central nervous system and peripheral nervous system including neurons, neuronal subtypes, oligodendrocytes, astrocytes, Schwann cells. Advantageously a viral vector may be selected from; adeno-associated virus (AAV), in particular AAV9, AAV1, 2, 4, 5, 6, 6.2, 8, 9, rh10, PHP.B, PH P.S, PH P.eB vectors can be used.

According to a third aspect of the present invention, there is provided a method of using a construct of the first aspect to express a transgene. Suitably the second aspect encompasses a method of expressing a transgene in a cell which may be provided to a subject. Suitably constructs can effectively be screened in vitro to assess the required level of dosage regulation. In vitro, the transgene can be contained within a plasmid and introduced into cell lines via lipid-mediated transfection. Robust transgene expression can be seen after 24 hours. Thereafter, the feed-forward transgene cassette suitably can be vectorized by insertion onto a rAAV expression vector which can then used to generate AAV particles.

According to a fourth aspect of the present invention, there is a method of treating a disorder caused by insufficient expression of a gene in a subject, the method comprising the steps of providing a construct of the first aspect of the invention or a vector of the second aspect with a wild type or codon optimised or modified copy of a transgene to be expressed in the subject to treat the condition caused by insufficient expression of the gene in the subject. Suitably, AAV viral vector packaged with the transgene will be introduced into the subject by various methods including systemic intravenous injection or by intra CSF routes of administration including intrathecal lumbar, intracerebroventricular, intra cisterna magna injection or by injection into neuropil.

Suitably the transgene may be a gene that is under-expressed in a subject who has the neurological disorder Rett Syndrome. Typically, Rett Syndrome is caused by loss-of-function mutations in the gene X-linked gene MECP2. Suitably, the transgene may be a functional copy or copies of the MECP2 gene. Suitably the construct provides for delivery of the transgene to the nervous system using adeno-associated virus (AAV) vectors.

The construct provides for expression of a transgene within a narrow/desired range in a target cell. For example where the transgene is a wild type or codon optimised copy of the protein coding sequence of the MECP2 gene, it is considered that the construct can provide the transgene at an expression level which provides a suitable therapeutic effect but which is less than a level at which adverse effects are observed. In the case of MECP2, FMR1 and UBE3A, overexpression of the gene is known to be deleterious.

For example, in Rett syndrome, the inventors have previously shown that low levels of expression can ameliorate disease phenotypes in mice. Conversely, overexpression (duplication of the gene locus) in patients, as well as in experimental animals, (2× or more) result in adverse neurological outcomes. This defines a narrow therapeutic window for genetic therapy for which the feed forward technology is well suited. The FMR1, UBE3A and SYNGAP1 genes are also considered to be dosage sensitive. In such circumstances, the level of expression of the transgene to ameliorate disease, but minimise adverse effects could be determined and then the expression level suitably provided to a patient using the present invention.

Many other genes associated with monogenic disorders are dosage sensitive and would benefit from use of a construct and system of the present invention to regulate expression of such exogenous transgenes. Human copy number variants (CNVs) can be an indication of dosage sensitive genes, and studies have implicated the dosage sensitivity of individual genes as a common cause of CNV pathogenicity. Gu W & Lupski JR. CNV and nervous system diseases—what's new? Cytogenet Genome Res. 2008; 123:54-64 cite several examples of dosage sensitive genes and their associations with neurodevelopmental disorders. Examples include MECP2 duplication syndrome (involving the gene MECP2), adult-onset autosomal dominant leukodystrophy (ADLD, involving the LMNB1 gene), isolated lissencephaly sequence (ILS, involving the PAFAH1B1/LIS1 gene), Miller-Dieker syndrome (MDS, involving the YWHAE gene).

Rice AM & McLysaght. Dosage sensitivity is a major determinant of human copy number variant pathogenicity. Nature Communications. 2017; 8:14366|DOI: 10.1038 show that solitary pathogenic genes involved in CNVs associated with disease are enriched for roles in neurodevelopment and identify many dosage sensitive genes, for example: PRKCZ, TTC34, PRDM16, ARHGEF16, PARK7, PRDM2, IGSF21, PTCH2, NFIA, ST6GALNAC3, DPYD, COL11A1, PDZK1, GPR89A, NBPF11, GPR89B, KCNT2, CFHR2, ASPM, PTPRC, GPATCH2, DUSP10, GPR137B, RYR2, CHRM3, RGS7, AKT3, KIF26B, SMYD3, LPIN1, EPCAM, MSH2, NRXN1, XPO1, LRP1B, ZEB2, ACVR2A, MBD5, KIF5C, SCN1A, COL3A1, PMS1, PLCL1, SATB2, PARD3B, EPHA4, SPHKAP, CHL1, GRM7, TRANK1, DOCK3, FAM19A1, FOXP1, ROBO1, CADM2, FOXL2, SOX2, LPP, RASGEF1B, GRID2, FAT4, NR3C2, LRBA, FGA, GALNTL6, WWC2, TLR3, IRX2, IRX1, CDH12, CDH9, NIPBL, HEXB, MEF2C, GRAMD3, FBN2, PRELID2, TCOF1, GABRG2, MSX2, NSD1, FOXC1, CDYL, TBC1D7, RUNX2, MUT, RIMS1, NKAIN2, LAMA2, ARID1B, PARK2, PACRG, QKI, TNRC18, FBXL18, SUGCT, GLI3, AUTS2, MLXIPL, COL1A2, PPP1R9A, CFTR, TSPAN12, GRM8, CNTNAP2, MNX1, CSMD1, MCPH1, LPL, ANK1, IMPAD1, CHD7, VCPIP1, TRPS1, PARP10, DOCK8, KANK1, GLIS3, PTPRD, MLLT3, ROR2, PTCH1, AL162389.1, ARRDC1, EHMT1, PCDH15, CTNNA3, ADK, BMPR1A, PAX2, BTRC, INPP5A, MRPL23, ELP4, PAX6, CPT1A, DYNC2H1, KIRREL3, WNK1, CACNA1C, PPFIBP1, TBX5, MED13L, NALCN, CHD8, MYH7, TTC6, DAAM1, NRXN3, MTA1, SNRPN, UBE3A, OCA2, HERC2, CHRFAM7A, ARHGAP11B, OTUD7A, FBN1, HEXA, SNUPN, NRG4, AC112693.2, IGF1R, LRRC28, HBA2, HBQ1, CREBBP, RBFOX1, CDR2, CDH13, CYBA, NXN, YWHAE, SMG6, METTL16, PAFAH1B1, ADORA2B, NT5M, RAI1, NF1, C17orf67, PITPNC1, ACOX1, TCF4, DOCK6, CACNA1A, LPHN1, ZSCAN5A, BMP2, MYT1, PEX26, USP18, DGCR6L, USP41, UBE2L3, NF2, LARGE, BRD1, SHANK3.

The inventors consider that any suitable gene, in particular any dosage sensitive gene, for example as discussed above, may suitably be utilised in the present invention as required. For example, as would be understood in the art, the constructs and systems of the present invention may be used in the expression of any suitably protein for treatment of a disease or a condition, particularly wherein control of the expression level of the protein being provided is of importance.

The inventors consider the concept, constructs with suitable transgenes therein and methods of expressing the transgene to be applicable to any other clinically relevant and dosage sensitive genes.

Suitably the construct may be used in other gene therapy programmes including Fragile X syndrome (using FMR1 transgene), Angelman syndrome (using for example UBE3A transgene), or Syngap-related intellectual disability (using SYNGAP1).

It can be envisaged that particular vectors may be used to provide a vector to a specific cell type dependent on disease.

For example, SYNGAP1 is a neuronal gene and expressed only in neurons, but UBE3A, MECP2 and FMR1 are ubiquitously expressed across multiple tissues. However, the dominant disease features occur in loss of expression in the nervous system and therefore the nervous system is the dominant target for the therapeutic feed-forward transgenes.

The inventors have developed constructs in which the synthetic components have been considered to fine-tune the system (number of sites and efficient intron exclusion) to achieve appropriate dosage-insensitivity. Dosage-insensitivity in the context of the present invention is intended to infer a range of protein expression that does not result in undesired effects that are observed when there is too much expression of a therapeutic transgene, for example, two copies of the MECP2 gene in an individual are known to result in a severe MECP2 duplication syndrome, with symptoms as severe as Rett syndrome, in which MeCP2 levels are drastically reduced, or absent.

In embodiments, the construct can contain two elements that allow the transgene levels to be controlled. Suitably, the first element may be a micro RNA sequence contained within an intron located between the promoter and transgene. This micro RNA containing intron will be spliced out during pre-mRNA processing. The miRNA will then be processed to produce a mature miRNA capable of degrading its target transcripts. An important element of the design is that the miRNA is designed not to target the mammalian genome in order to prevent off-target effects. In some examples the miRNA can be insect-derived (e.g. one from the Lampyridae group, but any suitable insect or other suitable non-mammalian miRNA could be optimized for this use). In alternative examples the sequence can be completely synthetic (designed such that it does not bind to the mammalian genome and is not a naturally occurring sequence) and is therefore devoid of known off-target effects within the mammalian genome. The second element can be a number of non-mammalian or synthetic miRNA binding sites in the 3′UTR of the construct that match the miRNA produced from the intron. The presence of these binding sites causes the transgene to be a target for the delivered micro RNA. This leads to reduced levels of the transgene and prevents overexpression, providing for the desired dosage insensitivity effect of the system.

In a separate embodiment of the feed-forward principle, the synthetic micro RNA is delivered within the gene therapy synthetic cassette intron, but instead of targeting a miRNA binding site contained within the 3′UTR, it is targeted against the coding sequence of the transgene itself. Crucially, in such an embodiment the transgene sequence is codon optimised such that the sequence is altered at the DNA level while remaining the same at the amino acid level. This creates a novel DNA sequence that allows synthetic miRNAs to be uniquely targeted to the transgene without targeting endogenous mammalian sequences. This version of the feed-forward system, being more compact, is advantageous for larger genes (for example Syngap1) which approach the packaging capacity of the viral vector. Overall, the single gene loop enables constant levels of expression whereby the circuit can maintain a relatively fixed level of expression across a broad range of gene dosages (i.e. this relatively fixed or constant expression level is what results in the desired dosage insensitivity). The experimental systems produced a regimen in which changes in gene dosage lead to much smaller relative changes in gene expression. This is an important feature when applied to gene therapy where one is aiming to achieve broad even expression across the transduced cell population and enables increased viral vector dosing to achieve higher transduction rates without concomitant overexpression effects.

In embodiments, the construct is suitable for expression in cells and/or tissues which are sensitive to AAV genetic therapy. In embodiments, the construct allows for control of transgene expression in cells which typically over-express transgenes delivered using AAV vectors. In embodiments, the construct prevents cellular toxicity in these cells and/or tissues. In embodiments, the construct may prevent cellular toxicity in dorsal root ganglions. In embodiments, the construct may prevent cellular toxicity in liver cells. In embodiments, the construct may prevent cellular toxicity in cardiac cells. In embodiments packaging of the construct in a viron does not affect or only minimally affects the quality of the construct.

In embodiments, the construct can be used to reduce the severity of clinical symptoms caused by certain genetic conditions or developmental disorders. In embodiments, the construct can be used to completely reverse clinical symptoms caused by certain genetic conditions or developmental disorders. In embodiments, the construct can be used to treat certain genetic conditions or developmental disorders. In embodiments, the construct can be used to treat Rett syndrome. In embodiments, the construct can be administered in vivo to reduce the clinical presentation of Rett syndrome.

In embodiments, the construct can be used to reduce toxicity of genetic therapy. In embodiments, the feed-forward mechanism regulates transgene expression, reducing the toxicity to cells. In embodiments, the construct can be administered in vivo without adverse health effects.

Embodiments of the invention will now be described by way of example only with reference to the accompanying figures in which:

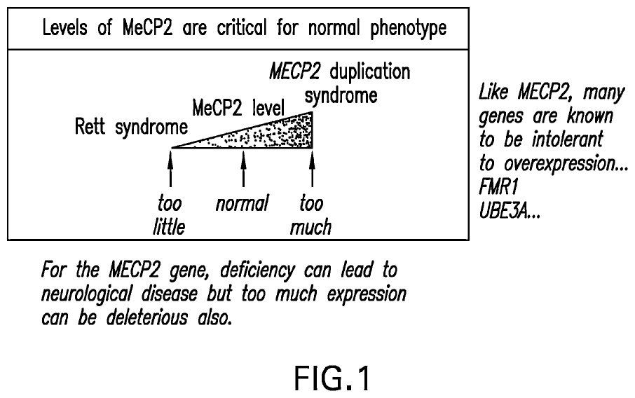

FIG. 1 illustrates challenges of dosage sensitivity in gene therapy.

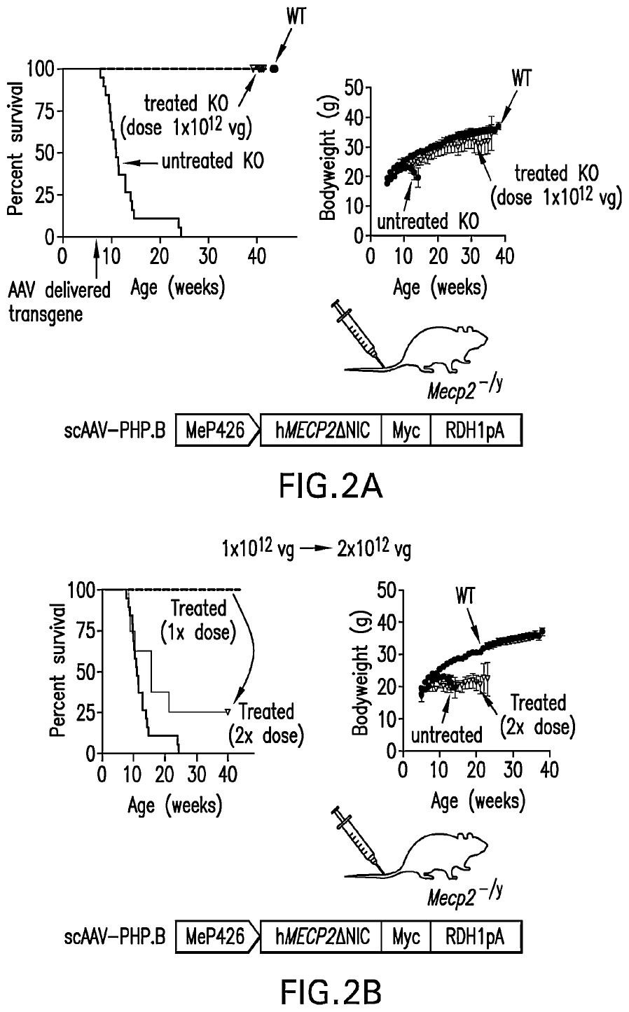

FIG. 2 illustrates gene dosage is a challenge in gene therapy and can result in very narrow safety windows. Gene dosage is a challenge in gene therapy and can result in very narrow safety windows. As an exemplar, mice modelling Rett syndrome have a median survival of ˜11 weeks. Treated with therapeutic gene therapy vector can normalise bodyweight and increase 40 week survival to 100% (left box). However, doubling this therapeutic dose results in lethality (right) highlighting dose sensitivity and narrow safety margin.

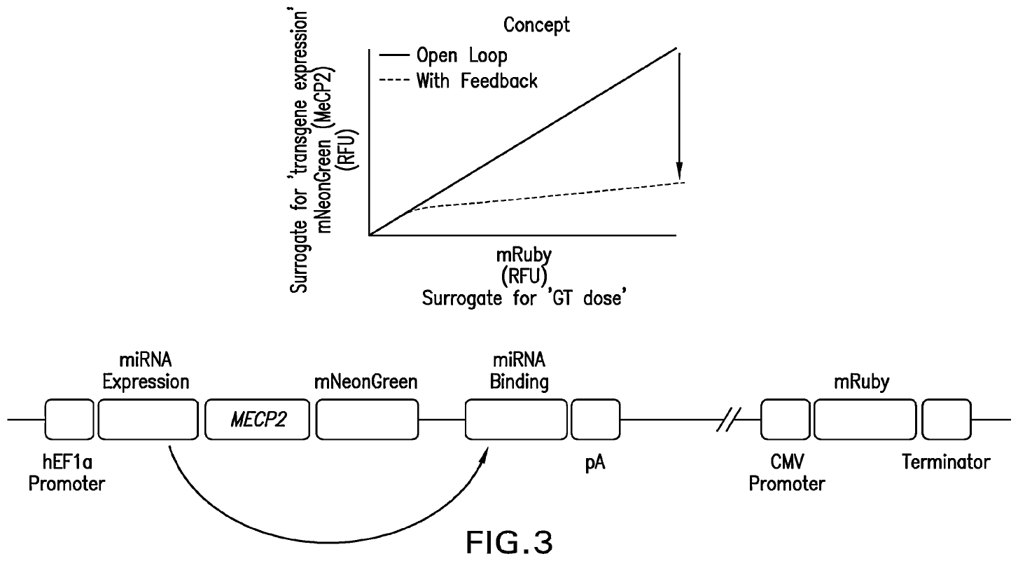

FIG. 3 illustrates a single gene feed forward gene therapy circuit can reduce dosage sensitivity as demonstrated by quantitative assessment of transgene levels using flow cytometry.

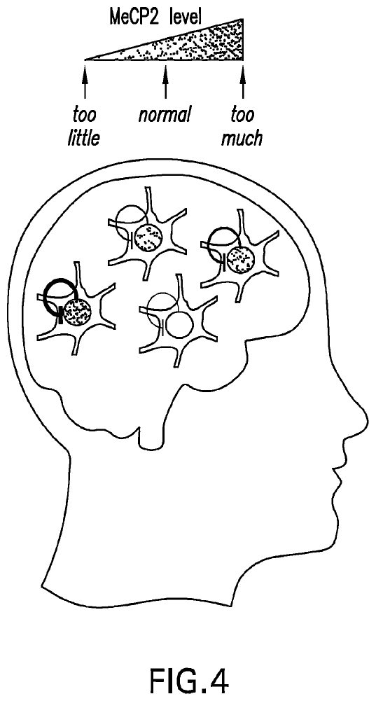

FIG. 4 illustrates feedback in relation to transgene expression provided by the level of virus of delivered transgene to any given cell, for example where cells are differentially infected and would otherwise express very different levels of the transgene. Feedback in relation to transgene expression provided by the level of virus of delivered transgene to any given cell. MECP2 is an example of a dosage sensitive gene with too little or too much causing disease. In gene therapy, cells receiving different levels of transduction will experience differential levels of feed-forward control (indicated by thickness of lines). Within the single gene circuit, the expression of the therapeutic transgene as well as its negative regulator (synthetic miRNA) are driven by the same input (levels of therapeutic vector entering the cell). Within increasing levels of input (levels of vector), the circuit achieves higher levels of miRNA mediated down-regulation. The result is that the circuit can maintain a more fixed level of transgene expression across the cell population. In the absence of such regulation (non-regulated gene therapy cassettes), cells express more varied levels of vector derived protein as shown by shading.

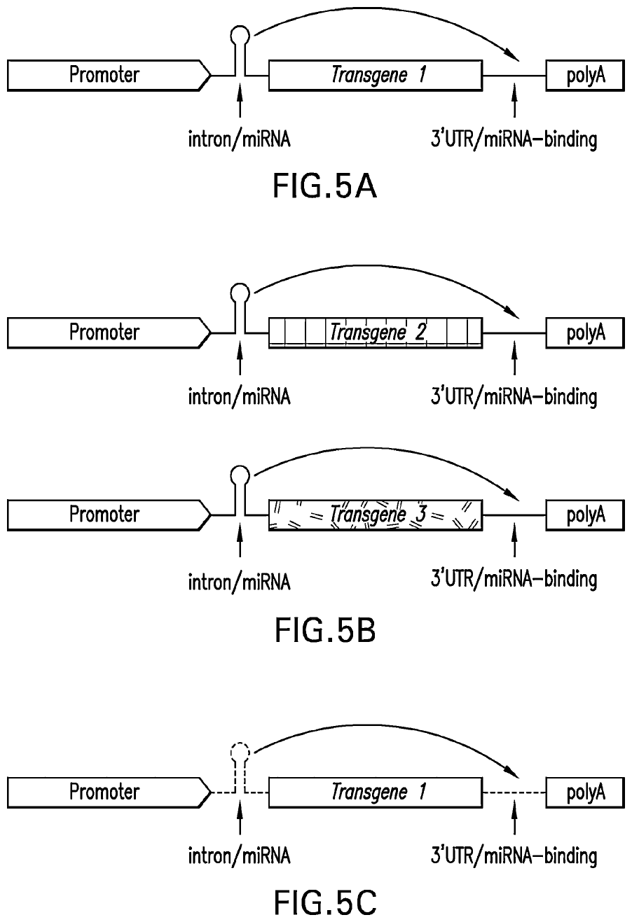

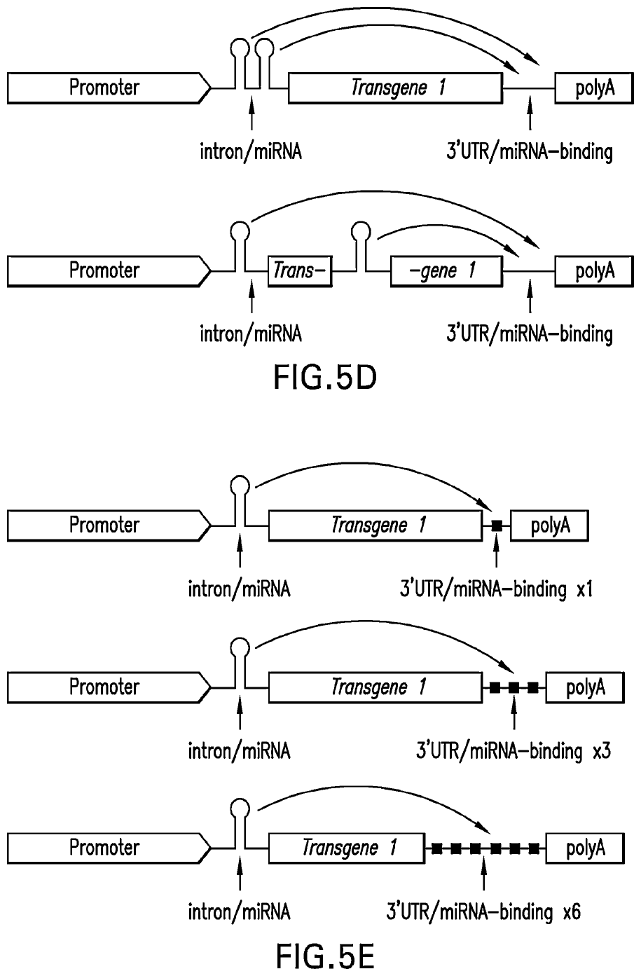

FIG. 5 illustrates the way in which the construct (cassette) can be optimised to treat different conditions utilising different transgenes or to provide different therapeutic levels of expression of a transgene—(A) Key components of a feed-forward construct. (B) The transgene component has been replaced but the rest of the cassette components have been maintained. (C) A new intron/miRNA and 3′UTR/miRNA-binding site (dashed lines) has been introduced but the rest of the cassette components have been maintained, (D) Two copies of the non-mammalian or synthetic miRNA may be expressed from within the same intron, or from two different introns. An intron may be positioned within the 5′UTR and/or within the open-reading-frame of the transgene. (E) The 3′UTR may contain one, three or six copies of the non-mammalian or synthetic miRNA binding site, or any number in between.

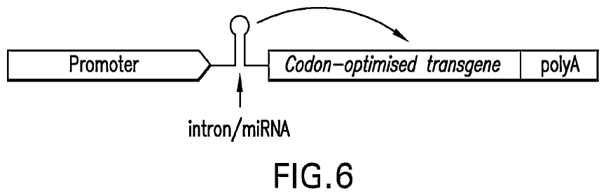

FIG. 6 illustrates a construct wherein the synthetic miRNA targets a sequence in the codon optimised transgene and not in the UTR.

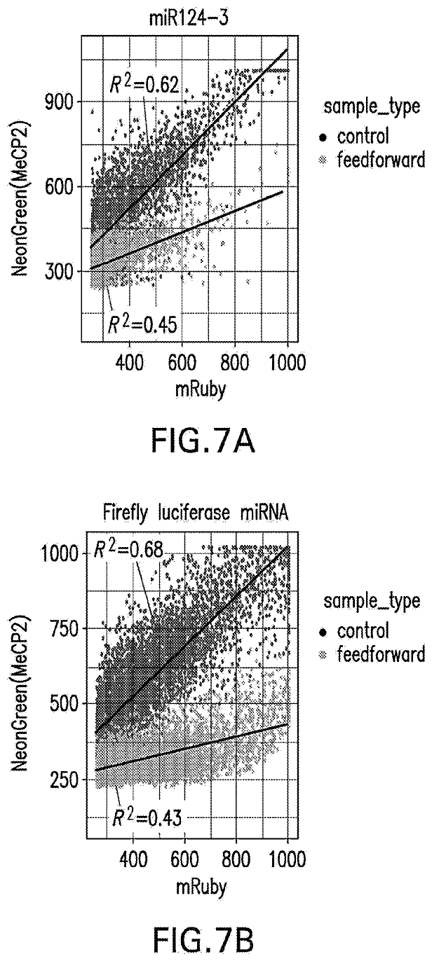

FIGS. 7A-B illustrates the effect of non-mammalian miRNA expression on MeCP2-NeonGreen protein levels as assessed by FACS. Demonstration of feed-forward using (A) native miRNA as well as (B) non-mammalian or synthetic miRNA devoid of predicted binding sites within the mammalian genome. Feedforward constructs (bottom line) were compared against control constructs (top line) which contained scrambled miRNA binding sites and therefore had no miRNA regulation (all the following experiments follow this same structure). Feedforward constructs contained 3 non-mammalian miRNA binding sites in the 3′UTR. Graphs show levels of mRuby (x-axis—measure of amount of plasmid to the cell and not affected by miRNA regulation) versus MeCP2-NeonGreen (y-axis—the protein regulated by the miRNA). The top graph shows results for miR124-3, an endogenous mammalian miRNA used in the feedforward circuits described in the Strovas publication of the art. The bottom graph shows results for ffluc1, a non-mammalian miRNA originally designed to knockdown firefly luciferase fluorescent protein. Results show that both miRNAs are effective in regulating MeCP2 expression in feedforward sample compared to controls as shown by the difference in slope of the linear regression lines.

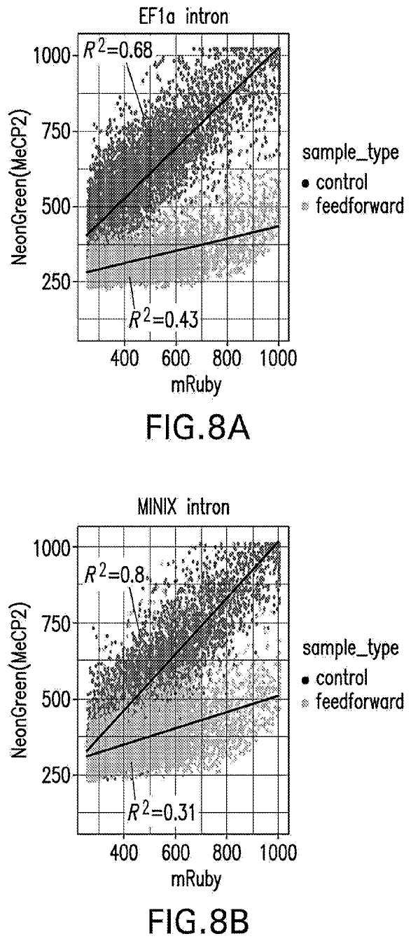

FIGS. 8A-B illustrates the non-mammalian miRNA. Examples of compact introns that can be incorporated into gene therapy cassettes and used to harbour non-mammalian or synthetic miRNA to achieve feed-forward control (in this and the following experiments the miRNA is the synthetic firefly luciferase (ffluc1) described in the previous figure) is expressed from an intron located between the promoter and MECP2 coding sequence. Robust expression of the non-mammalian miRNA relies on efficient splicing of this intron and the use of different introns could allow different levels of protein regulation. Feedforward molecules were made in which the non-mammalian miRNA was expressed either form intron 1 of the human EF1a gene or from a small synthetic intron (MINIX). Constructs contained 3 non mammalian miRNA binding sites in the 3′UTR. While both introns show robust regulation of MeCP2 levels, as seen by the reduced slope of the linear regression lines, the MINIX intron shows similar levels of MeCP2 expression to the control at lower levels of plasmid expression. It is considered that this is beneficial therapeutically as it will deliver therapeutic levels of protein at lower plasmid levels, but prevent protein toxicity at higher levels of plasmid delivery.

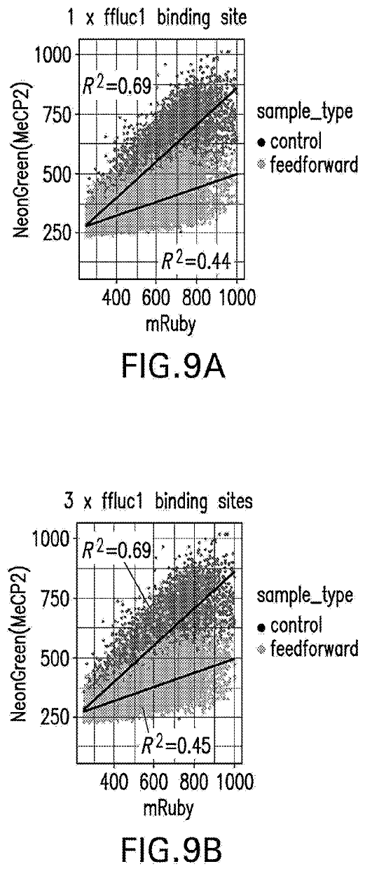

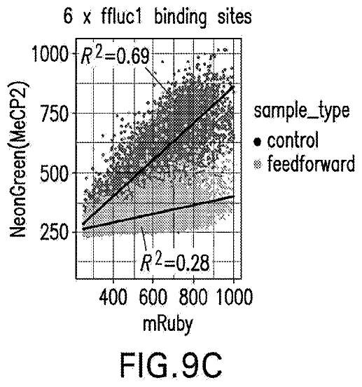

FIGS. 9A-C illustrates changing the number of non-mammalian miRNA binding sites in the 3′UTR. Three different constructs were made with either 1, 3, or 6 non mammalian miRNA binding sites in the 3′UTR and assessed by FACS. Constructs with 3 or 6 binding sites showed more significant repression of MeCP2 levels as shown by the reduced slope of the linear regression line. The strength of feed-forward control and thus dosage insensitivity can be fine-tuned by altering the number of non-mammalian or synthetic miRNA binding sites.

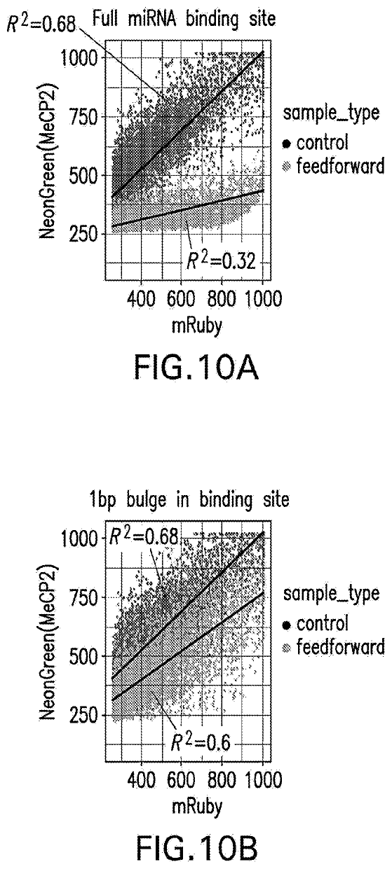

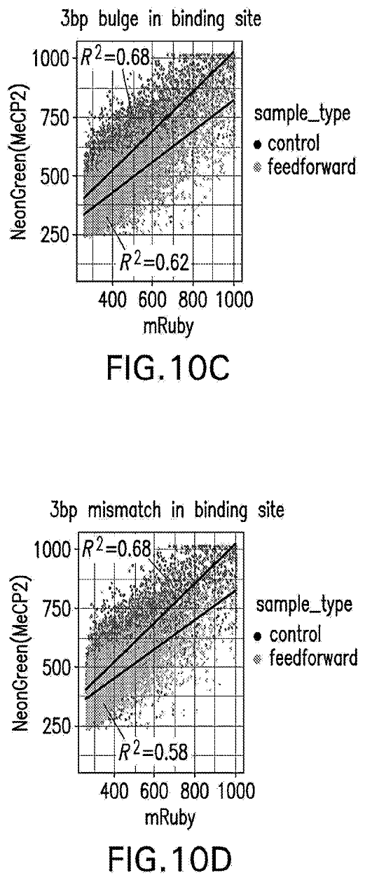

FIGS. 10A-D illustrates the effect of mismatches in the non-mammalian miRNA binding sites wherein three different constructs with either a 1 bp central bulge, a 3 bp central bulge, or a 3′mismatch in which only the miRNA seed sequence was present in the binding site. Compared to constructs with unmodified binding sites, these constructs showed markedly less repression of protein levels, with all three showing similar levels of repression. The strength of feed-forward control and thus dosage insensitivity can be fine-tuned by incorporating mismatches within non-mammalian or synthetic miRNA binding sites.

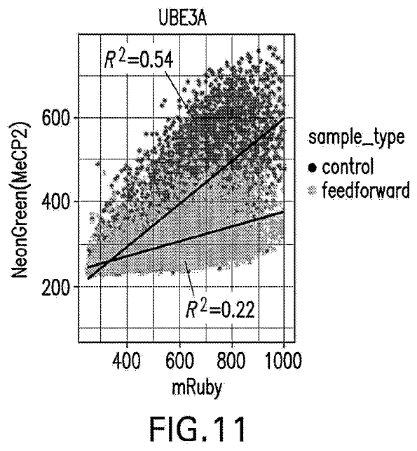

FIG. 11 illustrates whether the non-mammalian miRNA feedforward mechanism was also effective in other relevant brain disorders, wherein constructs were made with MECP2 replaced with the coding sequence for the UBE3A protein (mutations in this gene lead to Angelman Syndrome). The 3′UTR contained 3 non-mammalian miRNA binding sites for the same ffluc1 miRNA used in previous experiments. Once again, plasmids with non-mammalian miRNA binding sites showed reduced protein expression compared to plasmids with scrambled miRNA binding site sequences. It was postulated that UBE3A protein levels may be partially regulated by endogenous cellular mechanisms, independently of our feedforward non-mammalian miRNA mechanism. The feed-forward control of dosage sensitivity can be achieved across other dosage sensitive genes, in this case the UBE3A gene disrupted in Angelman syndrome and Prader-Willi syndrome.

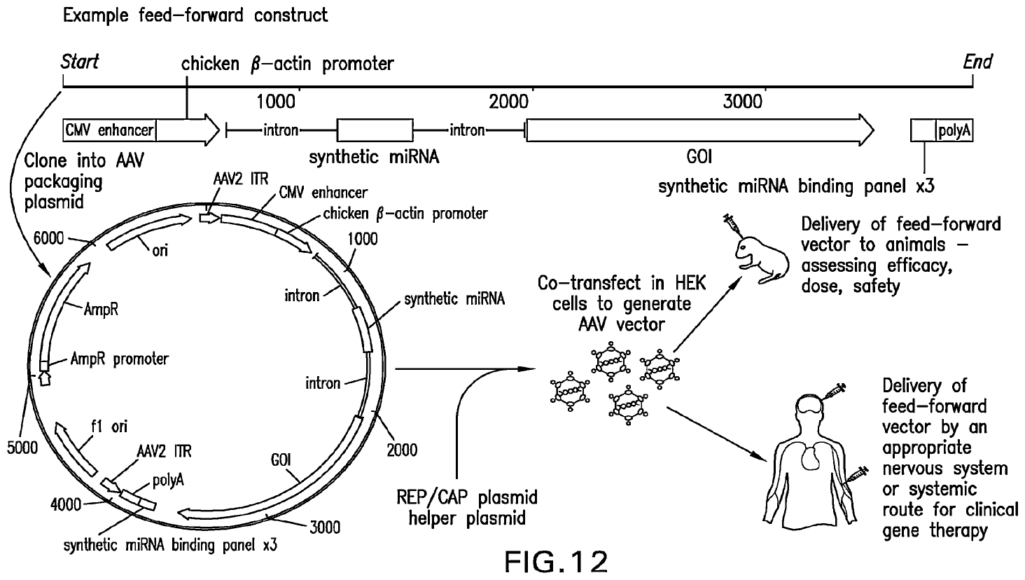

FIG. 12 illustrates the workflow in incorporation of feed-forward gene therapy technology, wherein feed-forward constructs are designed incorporating the appropriate assemblage of functional elements (see for example table 1 herein), are fabricated by DNA synthesis and then cloned into AAV packaging plasmid. The feed-forward cassette-bearing plasmid is then transfected alongside Rep/cap and helper plasmids to generate AAV particles for gene transfer therapy.

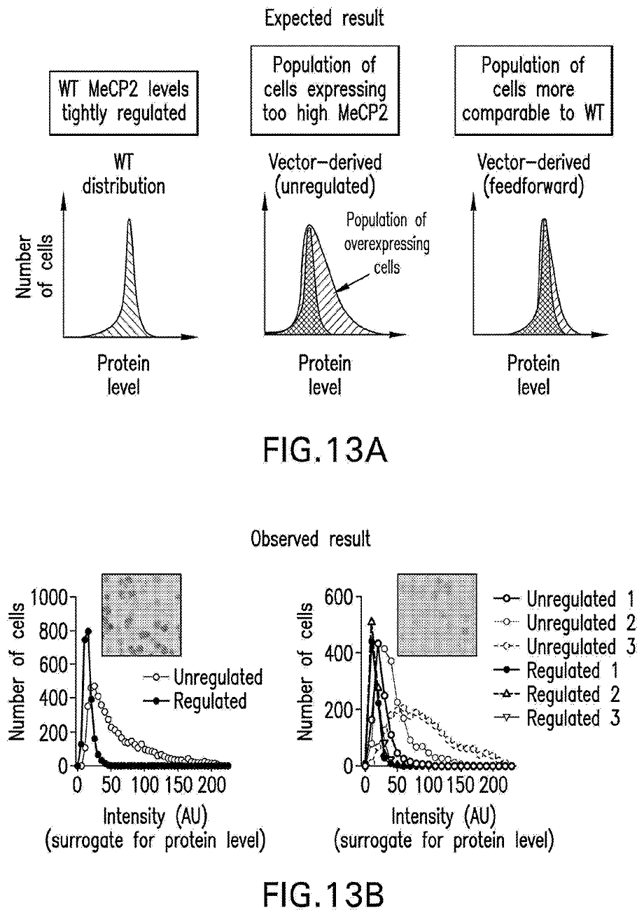

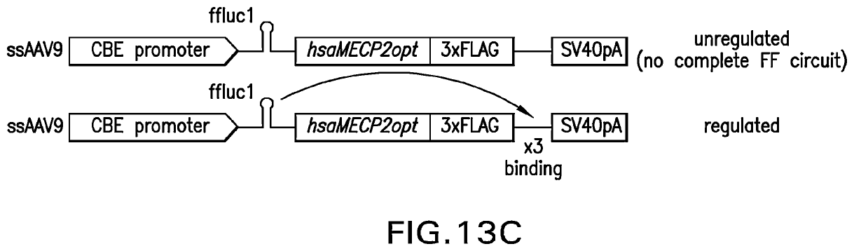

FIG. 13 illustrates the expression of MeCP2 after administration with a regulated cassette within the intact nervous system. 13A shows the predicted distribution of AAV vector-delivered protein expression. Wild-type distribution is represented as tightly regulated expression of native MeCP2 protein. Vector-derived (unregulated) distribution, with hatched area, shows a broad distribution of expression which afforded by the non-regulated cassette, including a significant proportion of cells expressing supra-physiological levels of protein. The vector-derived (feedforward) construct shows a hatched area which largely overlapping native distribution corresponding to constrained expression in the regulated cassette. 13B (observed result) shows fluorescence intensity imaging data (a surrogate for cellular protein level) from mouse brain somatosensory cortex at 12 days following AAV administration of control or feed-forward regulated vectors by direct brain injection. Mean data from 3 mice per treatment group is shown on left and individual animal data is shown on plot on far right. 13C shows a schematic diagram of the regulated and un-regulated feedforward AAV cassettes used in the experiment.

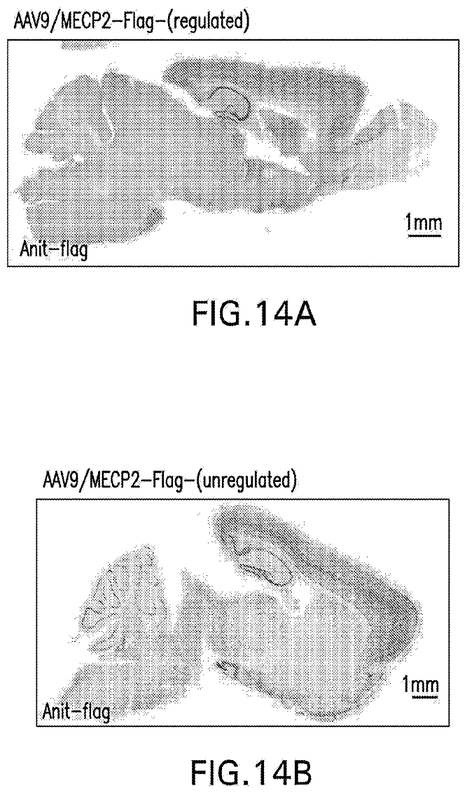

FIG. 14 illustrates brain wide expression of vector-derived protein from regulated and non-regulated AAV cassettes. The figure depicts tilted confocal images showing anti-flag tag immunolabeling (to detect vector-derived protein) of parasagittal mouse brain sections at 5 weeks post AAV injection.

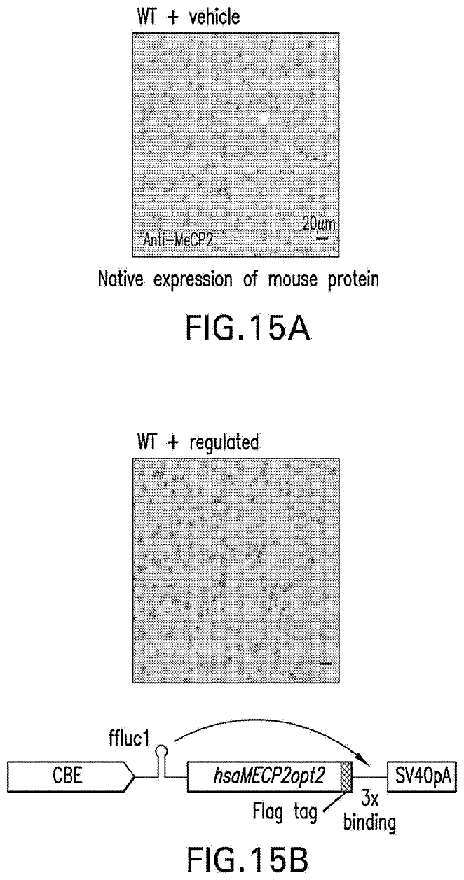

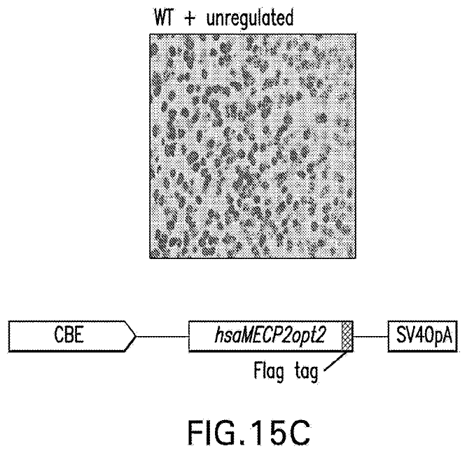

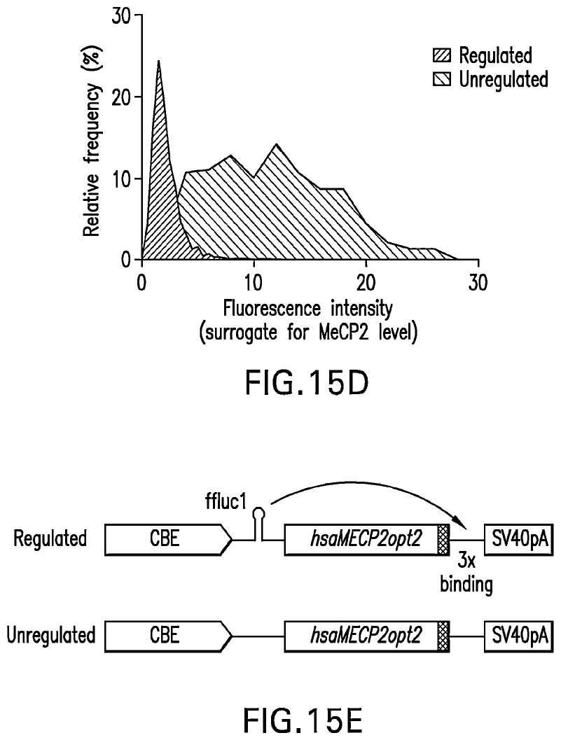

FIG. 15A-C illustrate fluorescent images showing constrained transgene expression as a result of the feedforward circuit. The images are representative confocal images showing anti-MeCP2 transgene immunolabeling (to detect vector-derived transgene product) of mouse somatosensory cortex at 5 weeks post AAV injection. Native levels of MeCP2 expression are shown in 15A. 15B shows MeCP2 immunoreactivity in wild-type (WT) mouse treated with regulated construct. 15C shows MECP2 immunoreactivity in WT mice treated with the unregulated construct. Schematics at the bottom show the feedforward regulated and non-regulated constructs. 15D shows the quantification of the vector-derived protein expression as measured by quantitative anti-Mecp2 immunolabeling. The expression is displayed as a relative frequency distribution (analysis of 1265-2082 cells per mice/cohort). Mice were injected with AAV vector at P1 at a dose of 1×10¹¹ vg/mouse. 15E shows a schematic of the regulated and un-regulated feed-forward constructs which were delivered to the mice.

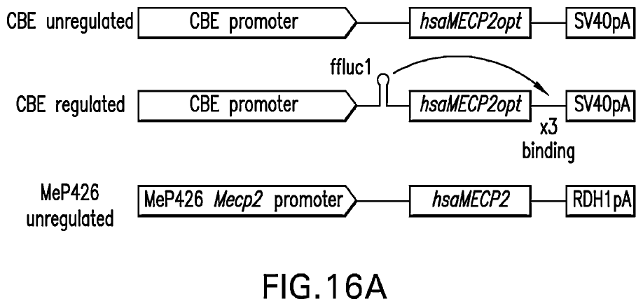

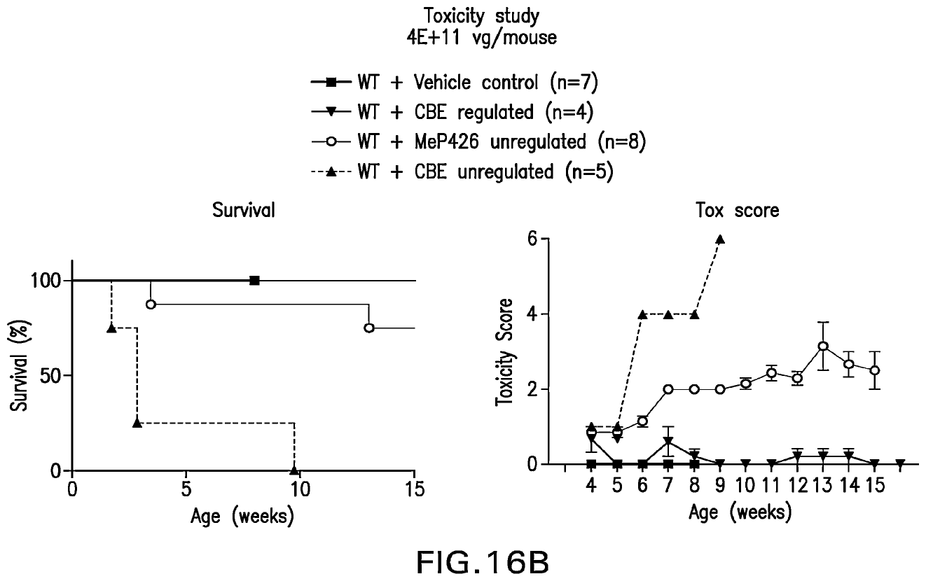

FIG. 16 depicts a toxicity study in which WT mice received an AAV9 dose of 4Ex10¹¹ vg/mouse. Regulated and un-regulated constructs which were tested are depicted in 16A. Survival and phenotype were tracked over a period of 15 weeks. The regulated construct confers safety advantages over the unregulated cassette. The figure shows an in vivo experiment in which wild-type mice were dosed with high dose vector (4×10¹¹ vg/mouse; direct brain injection at P1). The dosage with the unregulated MECP2 cassette, resulted in the development of a toxicity score and lethality. In contrast, regulated cassette was fully tolerated with no detectable overt deleterious phenotypes (16B).

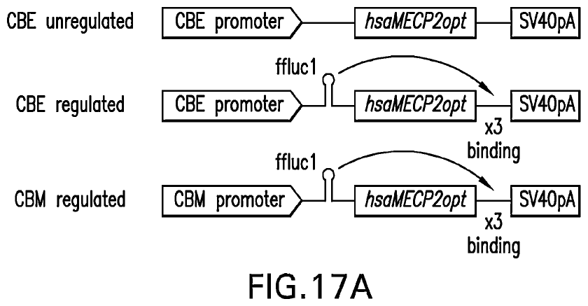

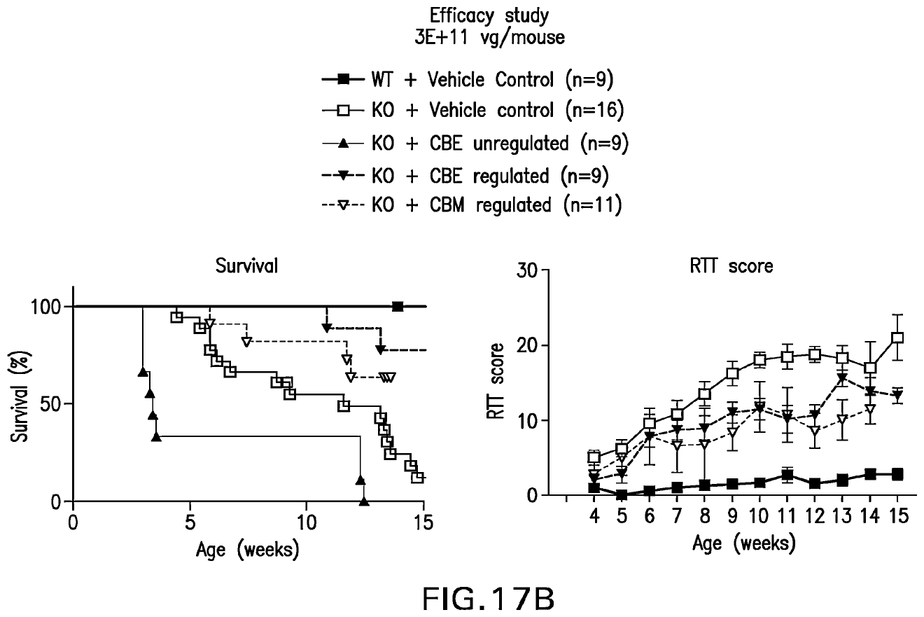

FIG. 17 demonstrates a study showing that administration of the regulated feed forward cassette is tolerated and showed a therapeutic effect in mice modelling Rett syndrome. In vivo experiment in which Mepc2^{-/y} mice were dosed with a high dosage of AAV9 vector (3×10¹¹ vg/mouse; direct brain injection at P1). Survival and phenotype (RTT score) were tracked over a period of 15 weeks (17B).

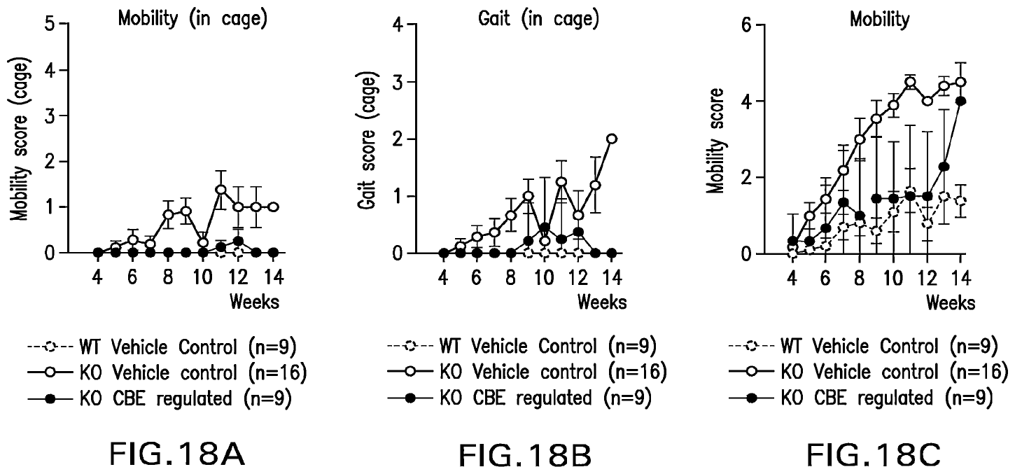

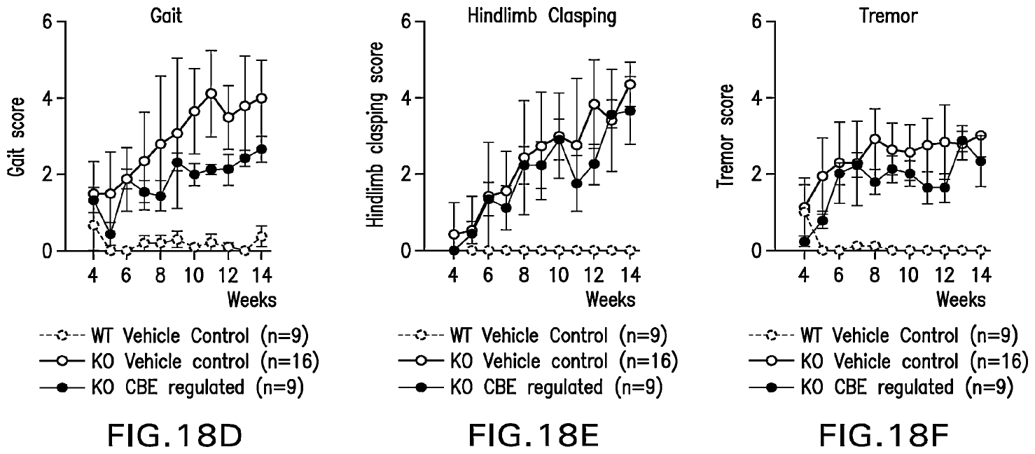

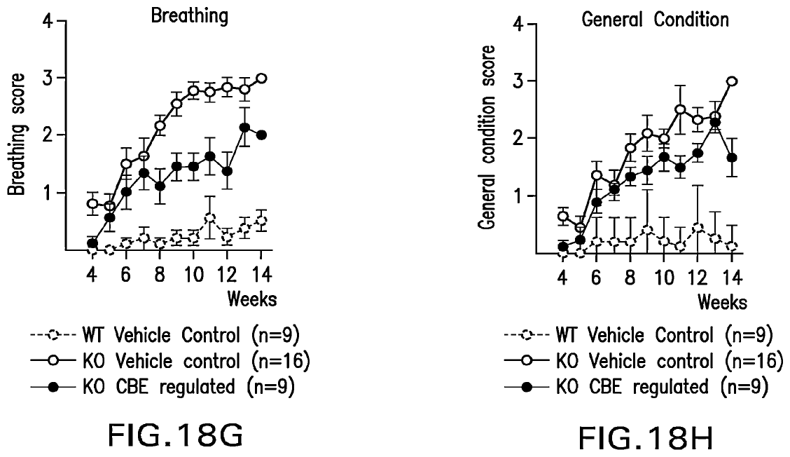

FIG. 18 illustrates that the regulated feed forward cassette normalises certain clinical features in mice modelling Rett syndrome. The figure shows an in vivo experiment in which Mepc2^{-/y} mice dosed with high dose of feedforward cassette (3×10¹¹ vg/mouse; direct brain injection at P1). Scoring for vehicle treated Mecp2^{-/y} mice and vehicle treated wild-type are shown for comparative purposes. Mice treated with non-regulated cassette at the same dose are not shown, as they did not survive monitoring period.

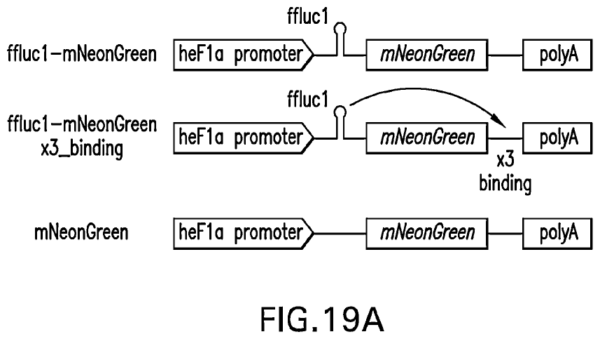

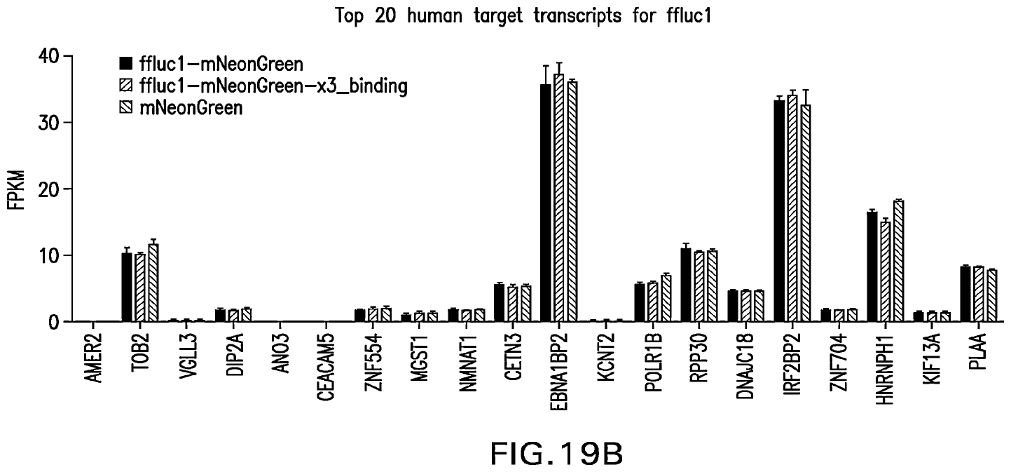

FIG. 19 illustrates RNAseq expression of the 20 genes which are considered to contain the most likely off-target interaction sequences for the miRNA ffluc1 used in the feed forward constructs. Plasmids expressing the ffluc1 miRNA and an mNeonGreen reporter transgene, or only the mNeonGreen reporter (19A). Expression levels of the top 20 predicted human target mRNA transcripts were measured using mRNAseq (19B). FPKM refers to the Fragments per Kilobase of transcript per Million reads. Low FPKM values indicate low levels of transcript abundance in human HEK 293 cells.

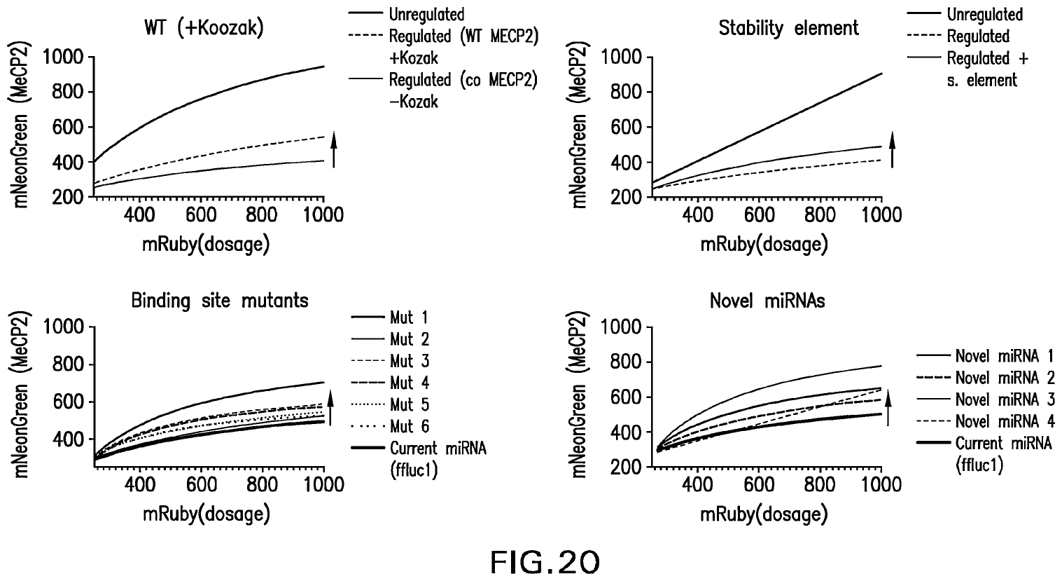

FIG. 20 illustrates the effect of transgene expression when additional elements (detailed in Example 8) are added to the feed forward cassette.

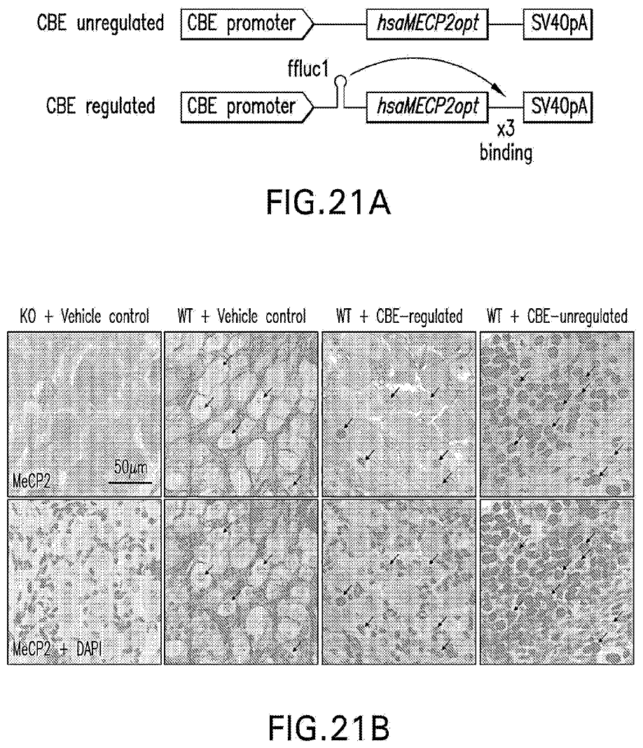

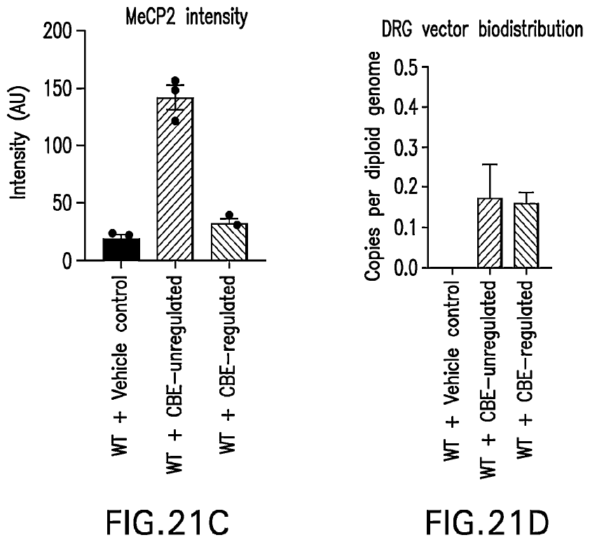

FIG. 21 details representative flattened confocal images taken from stained lumbar dorsal root ganglion (DRG) sections. Sections were cut 10 μm thick and stained with antiMeCP2 antibody and DAPI and imaged using identical confocal settings. 21A demonstrates the cassettes which were administered to the mice. 21B demonstrates the staining of the DRG sections from WT and Mecp2 knock-out mice treated with regulated and unregulated constructs. 21C shows quantification of the levels of MeCP2 as measured by fluorescence microscopy. 21D shows quantification of the number of copies of vector in each sample.

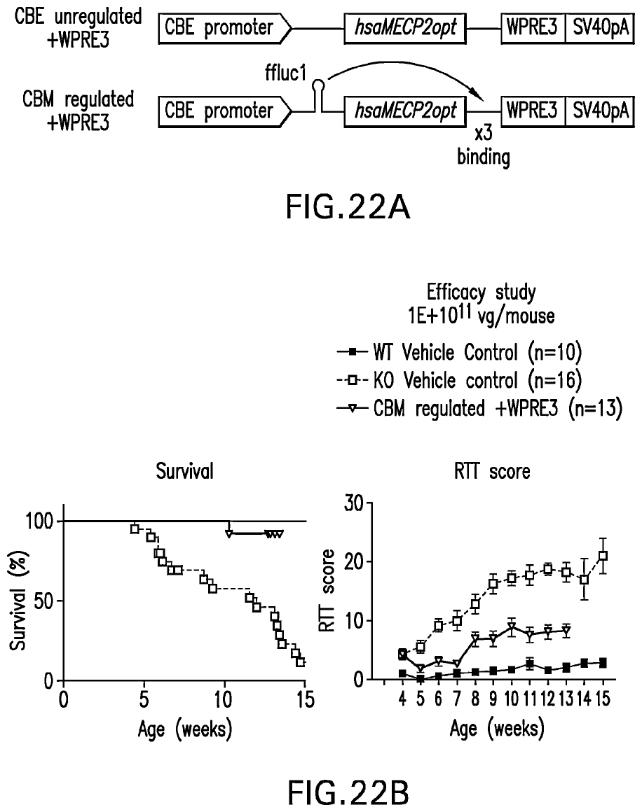

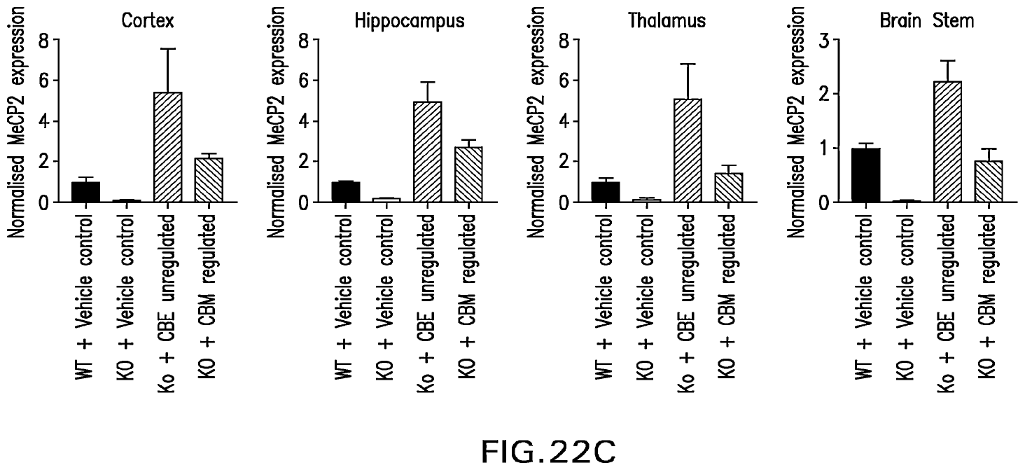

FIG. 22 shows an efficacy study in which Mecp2 KO mice received an AAV9 dose of 1Ex10¹¹ vg/mouse of AAV9 (22A). Survival and phenotype (RTT score) were tracked over a period of 15 weeks (22B). Western blot analysis of different brain regions demonstrates constrained MeCP2 expression with the feedforward circuit (22C).

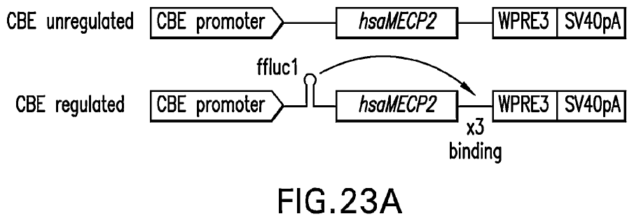

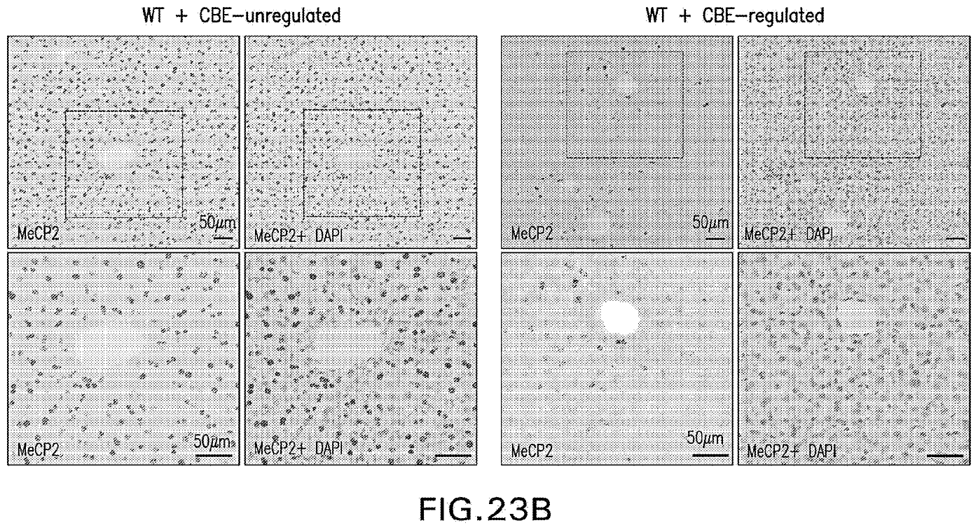

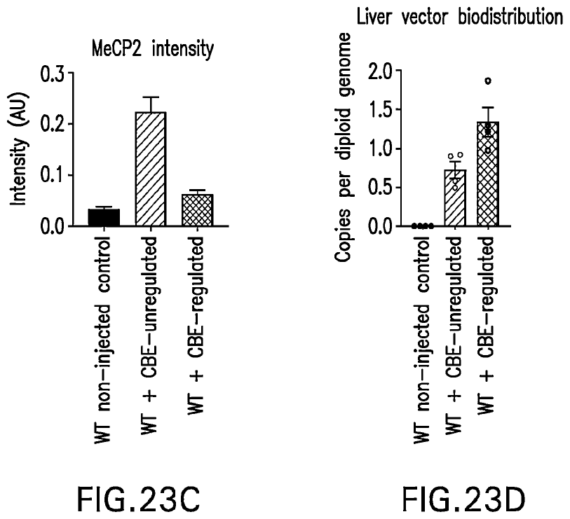

FIG. 23 details representative flattened confocal images taken from stained liver sections. Sections were cut 10 μm thick and stained with anti-MeCP2 antibody and DAPI and imaged using identical confocal settings. 23A demonstrates the cassettes which were administered to the mice. 23B demonstrates the staining of the liver sections from WT mice treated with unregulated and regulated constructs. Note that the regulated construct constrains expression of vector-derived transgene relative to non-regulated cassette. 23C shows quantification of MeCP2 levels as measured by intensity of fluorescent signal. 23D shows quantification of the number of copies of vector in each sample.

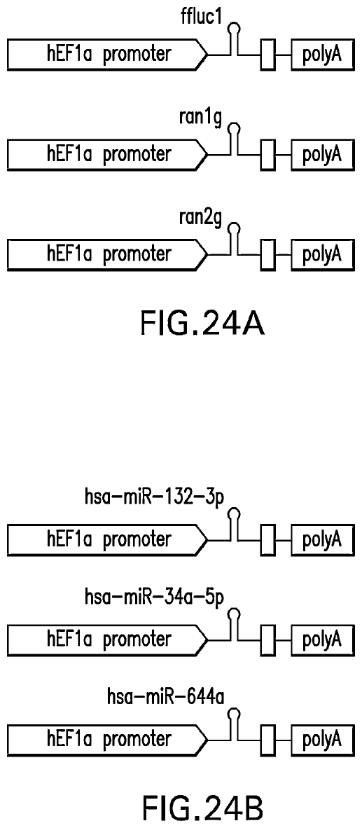

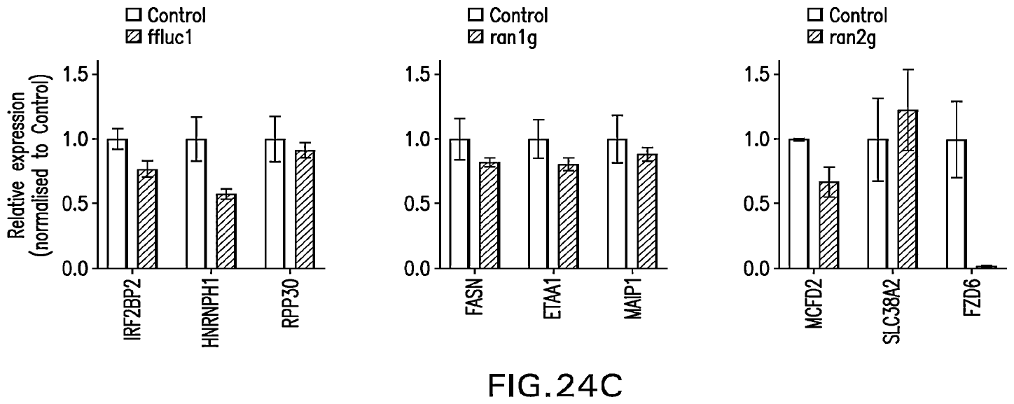

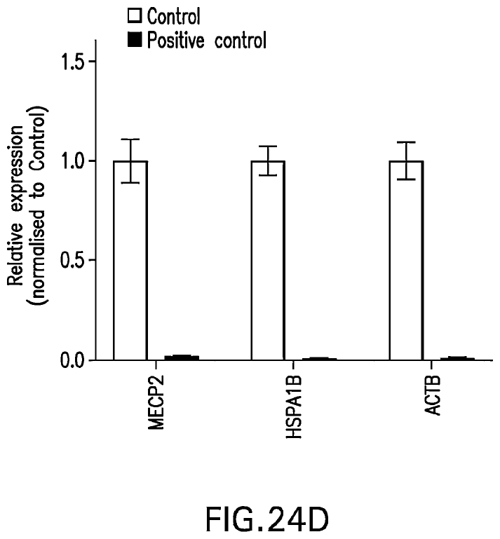

FIG. 24 illustrates qRT-PCR expression of mRNAs which are considered to be the most likely off-target interaction sequences for the miRNAs ffluc1, ran1g and ran2g used in the feed forward constructs. Plasmids expressing the ffluc1, ran1g or ran2g miRNA (24A). Control plasmids expressing the hsa-miR-132-3p, hsa-miR-34a-5p or hsa-miR-644a miRNA (24B). Expression levels of three top predicted human target mRNA transcripts were measured using qRT-PCR (24C). Expression levels of positive control human target mRNA transcripts were measured using qRT-PCR (24D).

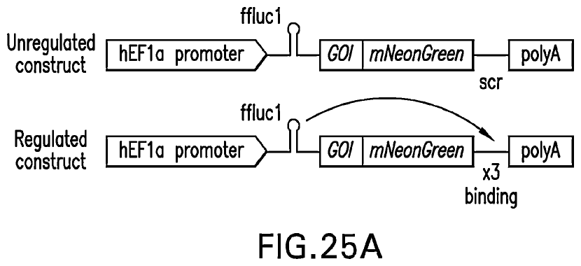

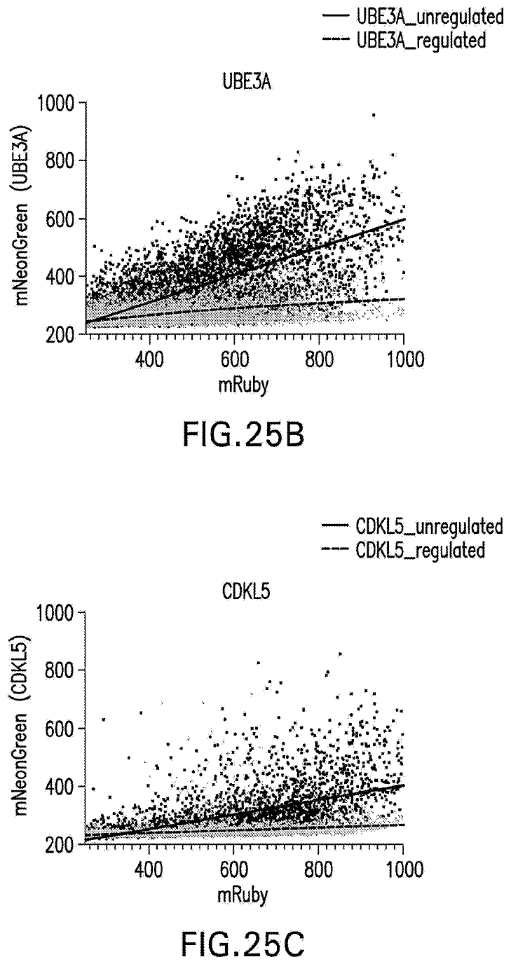

FIG. 25A-C shows that the feed-forward control of dosage sensitivity can be achieved across other dosage sensitive genes, in this case the UBE3A gene disrupted in Angelman syndrome and Prader-Willi syndrome (25B), and the CDKL5 gene disrupted in CDKL5 deficiency disorder (25C).

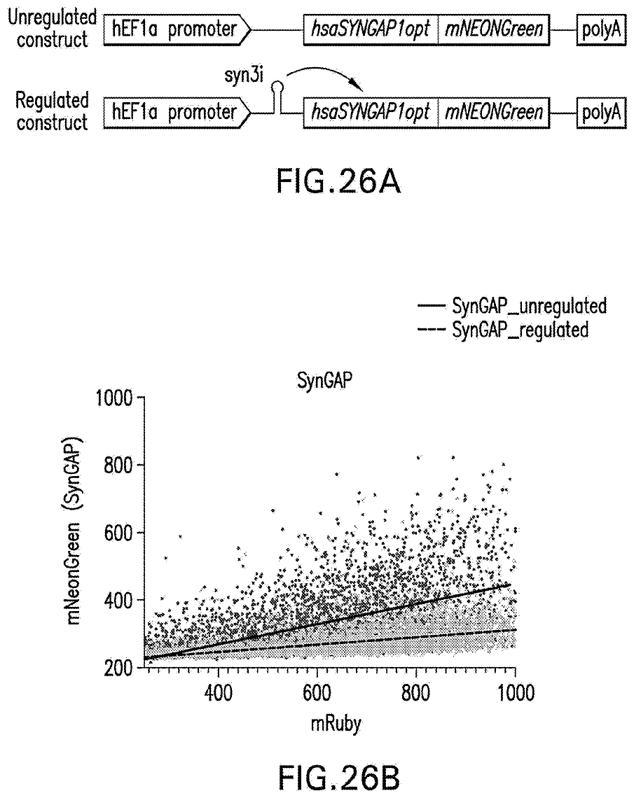

FIG. 26A-B shows that the feed-forward control of dosage sensitivity can be achieved across other dosage sensitive genes, in this case the SYNGAP1 gene disrupted in SYNGAP1-related intellectual disability, by a synthetic miRNA targeting a sequence in the codon optimised transgene and not in the UTR.

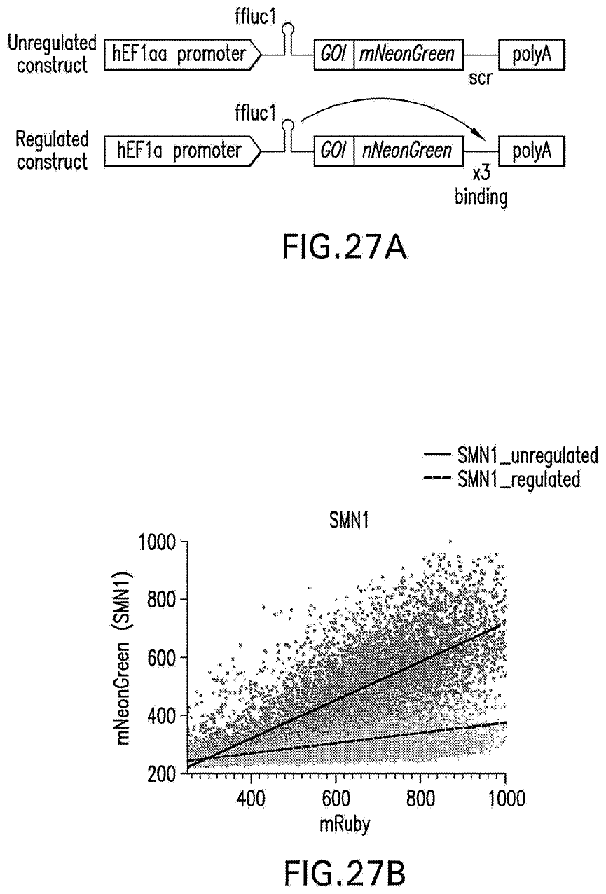

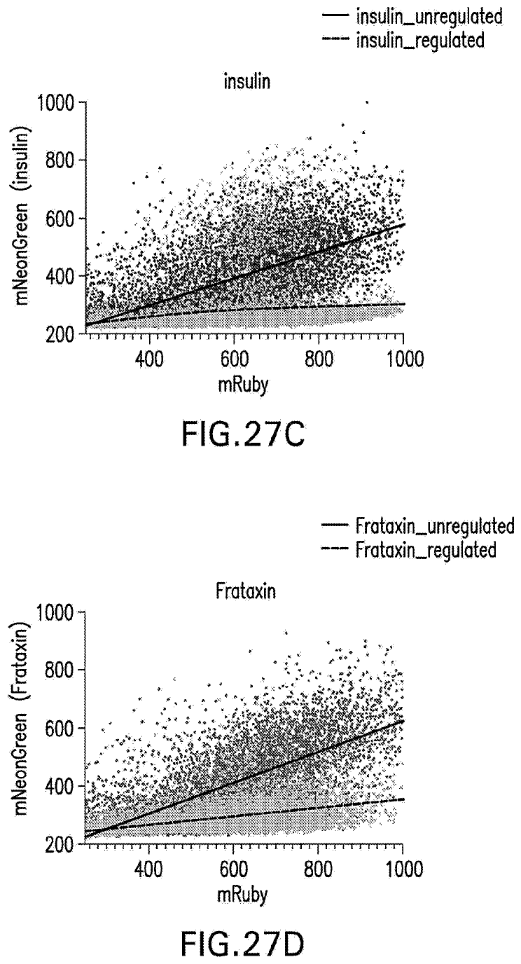

FIG. 27A-D The feed-forward control of dosage sensitivity can be achieved across other dosage sensitive genes, in this case the SMN1 gene disrupted in spinal muscular atrophy (27B), the INS gene disrupted in type 1 diabetes (27C) and the FXN gene disrupted in Friedreich's ataxia (27D).

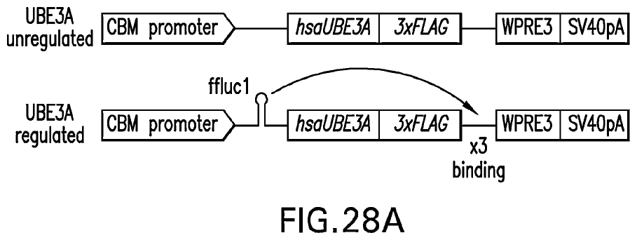

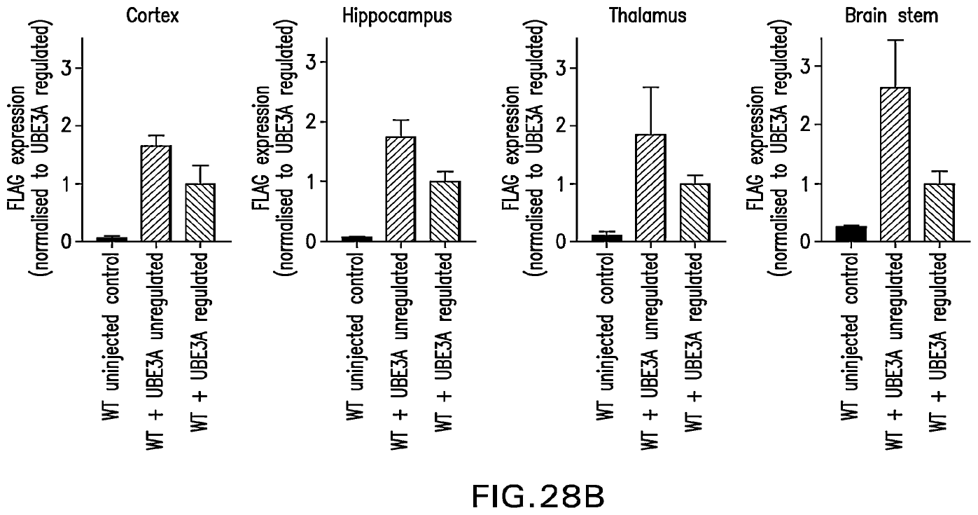

FIGS. 28A-B illustrates feed-forward control of dosage sensitivity can be achieved across other dosage sensitive genes in vivo, in this case the UBE3A gene disrupted in Angelman syndrome.

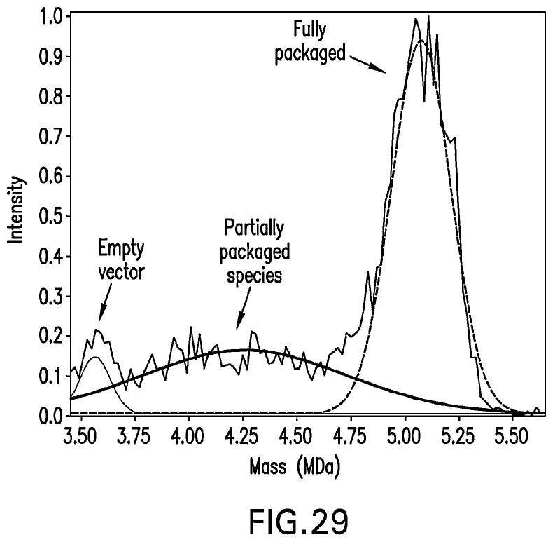

FIG. 29 shows CDMS data of a feed-forward MECP2 construct packaged in ssAAV9. Full-length feed-forward products package as desired, with low levels of aberrant or partial packaging. Secondary DNA structure, such as hairpins, are known to inhibit efficient packaging in AAV particles. However, in the feed-forward constructs analysed, the presence of miRNA hairpins (in the EF1a or MINIX intron) do not cause significant packaging of smaller than expected/partially packaged particles, and do not affect the quality of the AAV prep viron composition. The dominant peak corresponding to fully packaged MECP2 feed-forward cassette contrasts with the much smaller peaks representing empty particles and a distribution of partially packaged genome.

RTT253 construct:
CMV/CBA promote (no SEQ ID 76)
Human EF1a intron A (SEQ ID NO: 5)
ffluc1 (SEQ ID NO: 9)

### Kozak (SEQ ID NO: 73)

### Human MECP2_e1 (SEQ ID NO: 1)

ffluc1×3 binding sites (SEQ ID NO: 34)

### WPRE3 (SEQ ID NO: 75)

### SV40 pA (SEQ ID NO: 70)

### DETAILED DESCRIPTION

A proof-of-concept in the transgene targeting construct of the present invention has been generated in relation to the neurological disorder Rett Syndrome. Rett Syndrome is caused by loss-of-function mutations in the X-linked gene MECP2. Although an attractive therapeutic approach for this disorder is to deliver functional copies of the MECP2 gene to the nervous system using adeno-associated virus (AAV) vectors, a major obstacle to this approach is that cells can be infected with multiple copies of the virus vector leading to over-expression of the MECP2 gene. The inventors have previously determined that over expression of the MECP2 gene can lead to severe toxicity. Clinically it is known that duplication of the MECP2 gene in humans leads to MECP2 over-expression syndrome, a distinct and severe neurological disorder.

Using a construct as described by the present invention, the levels of MECP2 expressed in a cell can be limited, even when the cell has been infected with multiple copies of the viral vector. This greatly increases the safety window of MECP2 gene therapy interventions and allows higher viral doses to be administered, enabling a greater number of cells to be infected and a more robust disease reversal to be achieved.

In this example, the transgene is a WT or codon optimised copy of the protein coding sequence of the MECP2 gene, a gene mutated in the neurological disorder Rett Syndrome. The construct contains two elements that allow the transgene levels to be controlled. The first element is a non-mammalian or synthetic micro RNA sequence contained within an intron located between the promoter and transgene. This non-mammalian or synthetic micro RNA containing intron will be spliced out during pre-mRNA processing. The mammalian or synthetic miRNA will then be processed to produce a mature miRNA capable of degrading its target transcripts. As the miRNA is either synthetic or derived from a non-mammalian, insect source, it is therefore devoid of known off-target effects within the mammalian genome. A second element of the construct is a number of non-mammalian or miRNA binding sites in the 3′UTR of the construct that match the non-mammalian or synthetic miRNA produced from the intron. The presence of these binding sites causes the transgene to be a target for the delivered micro RNA. This leads to reduced levels of the transgene and prevents overexpression.

In an alternative embodiment of the feed-forward principle, the non-mammalian or synthetic micro RNA can be delivered within the gene therapy synthetic cassette intron. Instead of targeting micro RNA bindings within the 3′UTR, the non-mammalian or synthetic micro RNA instead binds to a unique (within the mammalian genome) micro RNA binding region that is created within the codon optimized protein coding sequence of the transgene, and has no corresponding binding site within the mammalian genome; i.e. the miRNA binding region is a unique synthetic binding region). This version of the feed-forward system, can be made more compact. This can be particularly advantageous for larger genes which approach the packaging capacity of a viral vector.

The single gene loop enables constant levels of expression whereby the circuit can maintain a relatively fixed level of expression across a broad range of gene dosages (i.e. exhibiting a desired dosage insensitivity). The experimental systems produce a regimen in which changes in gene dosage lead to much smaller relative changes in gene expression. This is an important feature when applied to gene therapy where one is aiming to achieve broad, even expression across the transduced cell population and enables increased dosing to achieve higher transduction rates without concomitant overexpression effects.

### EXAMPLES

### Example 1

Non mammalian miRNA binding sites or synthetic miRNA binding sites in combination with synthetic non mammalian miRNA (ffluc1) or synthetic miRNA which are not capable of binding to the mammalian genome can be utilised to ensure a lack of off-target effects, whilst enabling regulation of transgene expression. Suitably constructs as described by Table 1 may be provided.

TABLE 1
Summary of gene therapy constructs for lead indications and
the choice of feed-forward components based to empirical
testing and design constraints. These embodiments relate
to key dosage sensitive genes but, as will be appreciated
by those of skill in the art, the same feed forward design
can be applied to other dosage sensitive genes as would be
known or as determined in relation to specific conditions.

| | Pro- | | | | Binding | Poly |
|---|---|---|---|---|---|---|
| Disorder¹ | moter² | Intron³ | miRNA⁴ | Gene⁵ | sites⁶ | A⁷ |
| Rett | Jet | EF1a | ffluc1 | MECP2 | X 3* | sv40 |
| syndrome | | | | | | |
| Fragile X | CBA | EF1a | ffluc1 | FMR1 | X 3* | sv40 |
| syndrome | | | | | | |
| Angelman | CBA | EF1a | ffluc1 | UBE3A | X 3* | sv40 |
| syndrome | | | | | | |
| Syngap1- | Jet | Minix | ffluc1 | SYNGAP1 | X 1** | sv40 |
| related | | | | | | |
| NSID | | | | | | |

*binding sites within 3′UTR,
**binding sites within codon optimised transgene sequence.

As discussed, herein, the feed-forward system can be constructed using alternative ubiquitous and cell-type specific promoters including CAG, UBC, SV40, PGK, Synapsin1, neuron-specific enolase, U6, GFAP, MAG, MPZ. The intron may include any synthetic or endogenous intron capable of hosting the non-mammalian or synthetic miRNA sequence and may be upstream of the protein coding sequence or an intron within the protein coding sequence or a combination where more than a single non-mammalian or synthetic miRNA is generated from a single transgene cassette. The non-mammalian or synthetic miRNA may be any non-mammalian or synthetic miRNA that targets recognition sites within the transgene cassette including the translated and untranslated regions. The gene may be any dosage sensitive gene where gene dosage is confounding to the effectiveness of gene transfer. The number of binding sites may be fine-tuned to the level of desired dosage insensitivity and may range of 1, 2, 3, 4, 5, 6 or any number within the capacity of the transgene cassette. The polyA signal may suitably, for example be SV40, BGH or any commonly used native or synthetic polyA signal.

Neuro2a cells were transfected with various constructs, with or without the feed-forward mechanisms built-in, and the level of MECP2 transgene expression was assessed by flow cytometry. A separate fluorescent marker on the construct was used to monitor the level of construct delivered to each cell (surrogate for dose). Constructs in which the feed-forward control elements were included showed a much narrower range of MECP2 transgene expression than those which did not include these elements. Promisingly, the dampening effect of these elements increased as the amount of construct delivered increased suggesting that the control elements can mitigate toxicity without impeding expression of the gene at the therapeutic level. Fine tuning of the level of dosage sensitivity can therefore be provided.

### Example 2

The feedforward cassettes may be administered to mice to provide constrained transgene expression in cells. Wild-type mice had transgene flag tagged Mecp2 administered and transgene expression monitored in somatosensory cortex neurons. The transgene was delivered in an AAV vector which either did or did not contain a feedforward regulation system. The feedforward regulation system utilised miRNA ffluc1 (SEQ ID NO: 9) and EF1a promoter. Three ffluc1 binding sites (SEQ ID NO: 34) were provided after the Mecp2 sequence. FIG. 13C demonstrates a schematic representation of the viral vectors administered to the mice. The observed expression of MeCP2 in mice treated with the regulated (feed forward) and unregulated (no feed forward mechanism) cassettes. The regulated cassette consistently results in constrained expression (protein levels) and prevents the tail of cells expressing very high levels of vector-derived protein. Insets show representative micrographs from mouse brain expressing unregulated (bright but variable) and regulated (more even expression across cells). Suitably the feed forward mechanism can be used to ensure constrained protein expression for transgenes administered using viral vectors.

### Example 3

The feedforward regulation mechanism may be used to ensure appropriate distribution of transgene expression throughout a tissue. FIG. 14 demonstrates more consistent MeCP2-FLAG expression levels in regulated samples. The distribution of vector-derived protein is broad across both samples but that the regulated cassette is largely devoid of hotspots and gradients of expression relative to the unregulated version. Suitably, therefore, the feedforward mechanism may be used to control protein expression of a transgene at an appropriate concentration over a collection of cells, a tissue or an organ.

### Example 4

The feedforward regulation mechanism may be used to ensure constrained expression of a transgene throughout the neocortex. FIG. 15 demonstrates expression of native MeCP2 as compared to exogenous MeCP2 delivered to mice in AAV cassettes, with and without the feedforward regulation mechanism.

Single-stranded AAV (ssAAV) particles, comprising constructs flanked by AAV2 ITRs packaged into AAV9 capsids, were produced by transfection of HEK293 cells at the UPV Viral Vector Production Unit (Universitat Autònoma de Barcelona).

The miRNA utilised was ffluc1 (SEQ ID NO: 9), and 3×ffluc1 binding sites (SEQ ID NO: 34) were provided after the Mecp2 gene sequence. Expression is even across cells in the regulated image (15B) (but slightly higher due to combined native plus vector-derived signal), demonstrating constrained expression. In contrast, the unregulated cassette sample (15C) shows variable levels of immunoreactivity across cell population including populations of cells expressing very high levels of MeCP2. The quantification of these samples (15D) shows narrowly constrained expression with the feed forward cassette.

### Example 5

Suitably, the feed-forward cassettes may be administered in vivo without adverse health effects. Phenotypic assessment was carried out on wild-type mice administered with a feed-forward regulated cassette. Regulated constructs expressing the ffluc1 (SEQ ID NO: 9) miRNA and a codon-optimized human MECP2 transgene were administered. Unregulated constructs expressed only the codon-optimized human MECP2 transgene. The MeP426 unregulated construct expressed wild-type human MECP2 under the control of an endogenous mouse Mecp2 promoter, previously described by Gadalla K K E, Vudhironarit T, Hector R D, Sinnett S, Bahey N G, Bailey M E S, Gray S J, Cobb S R. Development of a Novel AAV Gene Therapy Cassette with Improved Safety Features and Efficacy in a Mouse Model of Rett Syndrome. Mol Ther Methods Clin Dev. 2017 Jun. 16; 5:180-190.

FIG. 16 depicts a high dosage study, showing there is constrained transgene expression with the feedforward circuit. This constrained transgene expression confers safety advantages over the unregulated cassette. The figure shows an in vivo experiment in which wild-type mice are dosed with high dose vector (4×10¹¹ vg/mouse; direct brain injection at P1). The dosage with the unregulated MECP2 cassette (16A) resulted in the development of a toxicity score and lethality (16B). In contrast, regulated cassette was fully tolerated with no detectable overt deleterious phenotypes.

### Example 6

Suitably, the feed forward mechanism does not interact with other sequences in the mammalian genome.

The miRNAs expressed in the feed-forward constructs, either insect derived miRNA sequence (ffluc1; SEQ ID NO: 9) or novel synthetic miRNA sequence (ran1g; SEQ ID NO: 17 and ran2g; SEQ ID NO: 18), have no predicted endogenous targets within the mammalian transcriptome.

To verify this, the mirDB off-target prediction tool was used to predict the most likely human mRNA targets of the miRNA sequences ffluc1, ran1g and ran2g. Potential human target genes/transcripts were ranked based on the number of target sites in the gene/transcript sequence matching the seed sequence of the miRNA.

Plasmids were generated that expressed the ffluc1 miRNA and a reporter transgene (FIG. 19A). The plasmids contained an hEF1a promoter driving expression of an mNeonGreen reporter transgene. The ffluc1 miRNA was expressed within the EF1a intron, situated between the hEF1a promoter and the transgene. HEK 293 cells were transfected with 100 μg of each plasmid using Lipofectamine. After 48 hrs, cells were lysed and total RNA isolated using the MagMAX-96 Total RNA Isolation Kit (Thermo Fisher). Samples were pooled to generate three biological replicates for each test plasmid. RNAseq was performed on each biological replicate and read counts (FPKM: Fragments Per Kilobase of transcript per Million reads) used to compare expression levels of individual human target transcript.

FIG. 19 depicts an analysis of the top 20 predicted human mRNA targets of ffluc1, showing there were no significant difference in the expression levels between sample sets and controls. The results confirm that over-expression of ffluc1 does not have off-target effects in any predicted human target genes.

Suitably, therefore, the invention provides a method of regulating transgene expression without impacting upon endogenous gene expression in a mammalian host cell.

### Example 7

Suitably, the feed forward mechanism can be used to provide safe and effective treatment to ameliorate the phenotype of clinical conditions.

AAV vectors expressing feed-forward MECP2 constructs were tested in wild-type (WT) and Mecp2 knock-out (KO) mice maintained on a mixed CBA/C57 background. ssAAV expressing regulated (ffluc1; SEQ ID NO: 9) or unregulated MECP2 was injected bilaterally into the brains of postnatal day (P)0/1 males by intracerebroventricular (ICV) administration. Control injections used the same diluent without vector (vehicle control). Injected pups were returned to the home cage and assessed weekly from 4 weeks of age. Mice were monitored until 15 weeks of age, or until reaching their human endpoint. FIG. 17B shows the clinical scores and survival of the WT and KO mice under all treatment conditions. Dosage with the unregulated MECP2 cassette resulted in toxicity and reduced survival. In contrast, dosage with the regulated cassette was fully tolerated and mice presented a less severe clinical score (for Rett-like phenotypes) compared to the mice treated with an unregulated feed-forward cassette or vehicle. The regulated mechanism therefore shows both safe administration and correction of the clinical severity of the phenotype in KO mice.

FIG. 18 further expands upon this data, measuring specific clinical features seen in mice modelling RETT syndrome. Administration of ffluc1 regulated cassette in Mecp2^{-/y} mice (KO) resulted in partial amelioration across of range of Rett-like phenotypes. This was not seen in KO mice treated with an unregulated construct, as mice did not survive to a stage where they could be phenotype tested.

### Example 8

Constructs can be provided wherein the constructs are modified to provide enhanced expression, regulation and stability. The constructs can be provided such that they contain a reporter transgene. The constructs can contain a Kozak sequence which promotes strong expression. The constructs can further contain a stability element in the 3′UTR. The constructs can further contain one or more binding sites which include mutations engineered to reduce the efficacy of (but not completely ameliorate) miRNA binding. Some exemplary constructs are detailed below in Table 2.

TABLE 2
Summary of gene therapy constructs designed to enhance transgene
expression whilst maintaining tight regulation of expression levels.
Elements of feed-forward mechanism based on empirical testing and
design constraints. These embodiments relate to key dosage sensitive
MeCP2, but, as will be appreciated by those of skill in the art,
the same feed forward design can be applied to other dosage sensitive
genes as would be known or as determined in relation to specific
conditions. It should be understood that any combination of the
features recited above may be used in the generation of a feed
forward construct. Further, it should be appreciated that these
constructs are exemplary and any of the above recited features
may be combined with any of the elements recited in Tables 3 and
4 to generate a feed forward cassette.

| | | | Gene of | Binding | Stability |
|---|---|---|---|---|---|
| Promoter | miRNA | Kozak | Interest | element | element |
| Strong | SEQ ID | SEQ ID | hsaMECP2 | SEQ ID | — |
| | NO: 9 | NO: 73 | | NO: 34 | |
| Strong | SEQ ID | SEQ ID | hsaMECP2 | SEQ ID | SEQ ID |
| | NO: 9 | NO: 73 | | NO: 34 | NO: 74 |
| Strong | SEQ ID | SEQ ID | hsaMECP2 | SEQ ID | SEQ ID |
| | NO: 9 | NO: 73 | | NO: 44 | NO: 74 |
| Strong | SEQ ID | SEQ ID | hsaMECP2 | SEQ ID | SEQ ID |
| | NO: 18 | NO: 73 | | NO: 53 | NO: 74 |
| Strong | SEQ ID | SEQ ID | hsaMECP2 | SEQ ID | SEQ ID |
| | NO: 9 | NO: 73 | | NO: 41 | NO: 74 |
| Strong | SEQ ID | SEQ ID | hsaMECP2 | SEQ ID | SEQ ID |
| | NO: 17 | NO: 73 | | NO: 52 | NO: 74 |
| Strong | SEQ ID | SEQ ID | hsaMECP2 | SEQ ID | SEQ ID |
| | NO: 9 | NO: 73 | | NO: 34 | NO: 75 |
| Strong | SEQ ID | SEQ ID | hsaMECP2 | SEQ ID | SEQ ID |
| | NO: 9 | NO: 73 | | NO: 44 | NO: 75 |
| Strong | SEQ ID | SEQ ID | hsaMECP2 | SEQ ID | SEQ ID |
| | NO: 18 | NO: 73 | | NO: 53 | NO: 75 |
| Strong | SEQ ID | SEQ ID | hsaMECP2 | SEQ ID | SEQ ID |
| | NO: 9 | NO: 73 | | NO: 41 | NO: 75 |
| Strong | SEQ ID | SEQ ID | hsaMECP2 | SEQ ID | SEQ ID |
| | NO: 17 | NO: 73 | | NO: 52 | NO: 75 |

FIG. 20 demonstrates the effect of the additional elements described above have upon MeCP2 expression in a regulated cassette. The assorted features demonstrate an influence on level of transgene expression relative to the dosage of cassette administered to HEK293T cells.

Any suitable promoter, constitutive or conditional, can be used to drive expression of the transgene. Suitably a promoter may comprise an Ef1a promoter, CAG promoter, Jet promoter, CMV promoter, CBA promoter, CBH promoter, Synapsin1 promoter, Mecp2 promoter, U1a promoter, U6 promoter, ubiquitin C promoter, neuron-specific enolase promoter, oligodendrocyte transcription factor 1 or GFAP promoter. It should be understood for the constructs Table 2, any suitable promoter may be used.

The miRNA used may be any suitable synthetic miRNA which does not bind to the mammalian genome. Suitably, the miRNA used may be derived from a synthetic sequence or a non-mammalian genome with no orthology to mammalian miRNAs. Suitably, the miRNA used may be derived from an insect genome. Exemplary miRNAs are provided in Table 3, below.)

TABLE 3
Sequences of miRNA elements which may be used in feed forward
constructs to regulate transgene expression. It will be understood
by the skilled person that any synthetic or non-mammalian miRNA
which can bind to a binding site, but which does not bind to
the mammalian genome, may be used. The effect of the varying
miRNAs upon transgene expression is demonstrated in FIG. 20.

| | | | Binding |
|---|---|---|---|
| | Alternative | | site |
| | name | miRNA | sequence |
| miRNA | (FIG. 20) | SEQ ID NO: | SEQ ID NO: |
| ffluc1 | Current miRNA | 9 | 34 |
| ran1g | Novel miRNA 1 | 17 | 52 |
| (novel_seq_5) | | | |
| ran2g | Novel miRNA 2 | 18 | 53 |
| (novel_seq_6) | | | |
| novel_seq_7 | Novel miRNA 3 | 19 | 54 |
| novel_seq_8 | Novel miRNA 4 | 20 | 55 |

The construct may be adapted to include a modified Kozak sequence: Suitably, the modified Kozak sequence may be any Kozak sequence which includes a nucleic acid motif that functions as the protein translation initiation site. Suitably, the modified Kozak sequence may be any modified sequence which promotes an increase in translation. Suitably, the Kozak sequence may be GCCACCATGG (SEQ ID NO: 73). FIG. 20 displays the effect which using SEQ ID NO: 73 as the Kozak sequence has upon transgene expression.

In embodiments, the gene of interest can be any one of the following genes of interest: MECP2, FMR1, UBE3A, CDKL5, FXN, SMN1, or INS or a gene required to be supplied using genetic therapy for treatment of a genetic condition or developmental disorder. In particular, the gene of interest may be any gene which requires controlled expression when delivered to a subject to treat a genetic condition or developmental disorder.

Examples of binding mutations may be seen in Table 4 below.

TABLE 4
Sequences of exemplary binding mutants, which may be introduced
into binding sites to partially ameliorate miRNA binding.
FIG. 20 demonstrates the impact varying mutant miRNA binding
sites have upon transgene expression.

| Binding site mutation | SEQ ID NO: |
|---|---|
| Mut 1 | 39 |
| Mut 2 | 40 |
| Mut 3 | 41 |
| Mut 4 | 42 |
| Mut 5 | 43 |
| Mut 6 | 44 |

Suitably, a stability element to increase transgene expression may be included. Suitably, the stability element may be located in the 3′ UTR. Suitably this stability element may be the Woodchuck Hepatitis Virus (WHV) Posttranscriptional Regulatory Element (WPRE) (SEQ ID NO: 74). Suitably, the stability element may be a truncated version of WPRE retaining the stability element, but omitting the X-protein sequence, or a ribozyme stability sequence (WPRE3). FIG. 20 shows the impact the stability element WPRE3 (SEQ ID NO: 75) has upon transgene expression.

### Example 9

### Assessment of Constrained Gene Expression in AAV-Susceptible Tissue

### Dorsal Root Ganglia

Dorsal root ganglions (DRGs) are highly susceptible to AAV. DRGs are highly transduced after AAV delivery and can result in toxicity. To test if the feed-forward circuit dampened expression in these tissues, DRG were dissected from wild-type mice treated with CBE-regulated and CBE-unregulated MECP2 feed-forward ssAAV at a dose of 4×10¹¹ vg/mouse. Lumbar DRGs were processed for vector derived MeCP2 expression (n=3 per mice, 3 mice per group) and vector biodistribution (1 DRG per mouse, 3 mice per group). Mice treated with CBE-unregulated MECP2 were terminated at 3-4 weeks old due to toxicity/humane endpoint. Mice treated with CBE-regulated MECP2 were terminated at 20 weeks. DRGs were also isolated from age-matched WT and KO mice as controls.

Upon termination, mice were perfused with 4% paraformaldehyde (PFA) then tissues were dissected and post-fixed in 4% PFA overnight at 4° C. then stored in 30% sucrose until time of processing. Tissues were embedded in a mixture 30% sucrose and Optimal cutting temperature (OCT) compound on dry ice. Frozen tissue blocks were stored at −20° C. until time of sectioning. Cryostat sections were cut at 12 μm and mounted on coated histological slides, air dried for 30 minutes at room temperature, then stored at −20° C. until time of staining. Frozen slides were rinsed in 0.1 MPBS to remove the tissue-freezing matrix then antigen retrieval was performed in 10 mM sodium citrate buffer, 0.05% Tween-20, pH 6.0) for 30 minutes in a water bath at 85° C. After cooling the slides for 30 minutes at room temperature in the same buffer slides were rinsed in 0.3M PBS/Triton X-100 solution then incubated with 5% goat serum in 0.3M PBS/T solution for 1 hour at room temperature in a humidified chamber to block non-specific binding. Slides were then incubated with the primary antibody (monoclonal, mouse anti-MECP2, M7443, Sigma, 1:500) in a buffered solution, overnight at 4° C. in a humidified chamber. After rinsing (0.3M PBS/T solution), slides were incubated with the secondary antibody (Alexa Fluor® 488 goat anti-mouse (H+L), cell signalling, 1:500) for 2 hours at room temperature in a humidified chamber. After further rinsing in 0.3M PBS/T solution, slides were incubated in Hoechst 33342, DNA dye staining solution (1:2000 in 0.1M PBS) for 30 minutes at room temperature. Slides were sealed to coverslips using anti-fade mounting solution and nail polish, then imaged by confocal microscope.

FIG. 21 depicts a high dosage study showing there is constrained transgene expression in DRGs with a feed-forward circuit. This constrained transgene expression confers safety advantages over the unregulated cassette. The figure shows an in vivo experiment in which wild-type mice are dosed with high dose vector (4×10¹¹ vg/mouse; direct brain injection at P1). The dosage with the unregulated MECP2 cassette (21A) resulted in significant MeCP2 over-expression in DRGs, and the development of toxicity and lethality. In contrast, the regulated cassette was fully tolerated and showed significantly lower levels of MeCP2 expression in DRG. Vector biodistribution analysis showed that DRGs were transduced with similar levels of AAV in CBE-regulated- and CBE-unregulated-treated mice (FIG. 21D), confirming that the difference observed in MeCP2 levels in DRGs is due to the dampening action of the feed-forward circuit (FIG. 21B-C).

### Liver

The liver is also highly susceptible to AAV. Liver cells are highly transduced after AAV delivery and can result in toxicity. To test if the feed-forward circuit dampened expression in these tissues, liver was dissected from wild-type mice treated systemically (intravenous) with CBE-regulated or CBE-unregulated MECP2 feed-forward ssAAV at a dose of 1×10¹² vg/mouse. Liver was processed for vector derived MeCP2 expression (n=3 sections per mouse, 3 mice per group) and vector biodistribution (3 mice per group). Mice treated with CBE-regulated and CBE-unregulated MECP2 were terminated at 4 weeks post-injection. Liver was also isolated from non-injected age-matched WT mice as controls.

Upon termination, mice were perfused with 4% paraformaldehyde (PFA) then tissues were dissected and post-fixed in 4% PFA overnight at 4° C. then stored in 30% sucrose until time of processing. Tissues were embedded in a mixture 30% sucrose and Optimal cutting temperature (OCT) compound on dry ice. Frozen tissue blocks were stored at −20° C. until time of sectioning. Cryostat sections were cut at 12 μm and mounted on coated histological slides, air dried for 30 minutes at room temperature (RT), then stored at −20° C. until time of staining. Frozen slides were rinsed in 0.1 MPBS to remove the tissue-freezing matrix then antigen retrieval was performed in 10 mM sodium citrate buffer, 0.05% Tween-20, pH 6.0 for 30 minutes in a water bath at 85° C. After cooling the slides for 30 mins at RT in the same buffer slides were rinsed in 0.3M PBS/Triton X-100 solution then incubated with 5% goat serum in 0.3M PBS/T solution for 1 hour at room temperature in a humidified chamber to block non-specific binding. Slides were then incubated with the primary antibody (mouse anti-MeCP2, 1:500) in a buffered solution, overnight at 4° C. in a humidified chamber. After rinsing (0.3 M PBS/T solution), slides were incubated with the secondary antibody (Alexa Fluor® 488 goat anti-mouse (H+L), 1:500) for 2 hours at room temperature in a humidified chamber. After further rinsing in 0.3M PBS/T solution, slides were incubated in Hoechst 33342, DNA dye staining solution (1:2000 in 0.1 M PBS) for 30 minutes at room temperature. Slides were sealed to coverslips using anti-fade mounting solution and nail polish, then imaged by confocal microscope.

FIG. 23 depicts a high dosage study showing there is constrained transgene expression in liver with a feed-forward circuit. This constrained transgene expression confers safety advantages over the unregulated cassette. The figure shows an in vivo experiment in which wild-type mice are dosed with high dose vector (2×10¹² vg/mouse; intravenous injection at 5.5 to 6.5 weeks old). This dosage with the unregulated MECP2 cassette (23A), resulted in significant MeCP2 over-expression in liver. In contrast, the regulated cassette showed significantly lower levels of MeCP2 expression in liver. Vector biodistribution analysis showed that livers were transduced with similar levels of AAV in CBE-regulated and CBE-unregulated-treated mice, confirming that the difference observed in MeCP2 levels in liver is due to the dampening action of the feed-forward circuit.

This further demonstrates that feed-forward constructs can constrain transgene over-expression even in tissues highly susceptible to AAV, reducing the probability of tissue damage/toxicity, and therefore providing an advantage over conventional gene therapy constructs.

Suitably, the feed-forward constructs can be used to constrain transgene over-expression even in tissues highly susceptible to AAV, reducing the probability of tissue damage/toxicity, and therefore providing an advantage over conventional gene therapy constructs.

### Example 10

Single-stranded AAV (ssAAV) particles, comprising constructs flanked by AAV2 ITRs packaged into AAV9 capsids, were produced by a baculovirus transfection system at Virovek (Hayward, CA, USA).

AAV vectors expressing modified feed-forward MECP2 constructs were tested in Mecp2 knock-out (KO) mice maintained on a mixed CBA/C57 background. ssAAV expressing regulated or unregulated MECP2 was injected bilaterally into the brains of postnatal day (P)0/1 males by intracerebroventricular (ICV) administration. Control injections used the same diluent without vector (vehicle control). Injected pups were returned to the home cage and assessed weekly from 4 weeks of age. Mice were monitored until 15 weeks of age, or until reaching their human endpoint.

FIG. 22 demonstrates a study showing that administration of the modified regulated feed forward cassette is tolerated and shows a therapeutic effect in mice modelling Rett syndrome (Mecp2 KO mice). The modified AAV-packaged construct (cassette) designs used in in vivo studies are illustrated (FIG. 22A). Regulated constructs expressed the ffluc1 miRNA (SEQ ID NO: 9) and a wild-type human MECP2 transgene. Unregulated constructs expressed only the wild-type human MECP2 transgene. MeCP2 protein expression was enhanced by the presence of a Woodchuck Hepatitis Virus Posttranscriptional Regulatory Element (WPRE3) (SEQ ID NO: 74) in the 3′UTR. Mecp2 KO mice were dosed with a vector (1×10¹¹ vg/mouse; direct brain injection at P1) and then assessed weekly from 4 weeks of age. Survival and RTT scoring data demonstrated that administration of the regulated feed forward cassette is tolerated and showed a therapeutic effect in mice modelling Rett syndrome (Mecp2 KO mice). Mice receiving the CBE-unregulated+WPRE3 construct had to be culled 2-3 weeks after injection due to severe overexpression toxicity.

Western blot analysis was performed (FIG. 22C) different brain regions (cortex, hippocampus, thalamus and brain stem). Frozen tissue samples were homogenised in a bead mill with 300 μl of buffer NE1 then stored on ice. After addition of 250 U benzonase nuclease to each sample, samples were shaken, incubated at room temperature for 15 minutes then stored on ice. Samples were diluted 1:20 in NE1 buffer for protein. 100 μl 4×Laemmli Sample Buffer was added to each bead mill tube, samples boiled for 10 min, then stored at −80° C. Samples were thawed and 25 μg amount of each sample migrated on a 10% acrylamide gel at 150 V until the dye front reached the bottom of the gel. Gels were then transferred to a nitrocellulose membrane for 2 hours at 85V. Total protein was measured. Total protein stain was removed, and membranes were incubated with LI-COR blocking buffer on a shaking incubator for 1 hour at room temperature. Membranes were then incubated overnight at 4° C. in 20 ml of LI-COR blocking buffer with primary antibody anti-MECP2 at a dilution of 1:1000. Membranes were washed in TBS-T buffer for 10 minutes (×3), and then incubated for 2 hours at room temperature in 20 ml of LI-COR® blocking buffer with secondary at a dilution of 1:10000. Membranes were washed in TBS-T buffer for 10 minutes (×3), rinsed with TBS buffer then imaged.

The results demonstrated constrained MeCP2 expression with the feedforward circuit. Combined with enhanced survival and a lower RTT phenotype score, this constrained transgene expression further demonstrates safety advantages over the unregulated cassette.

### Example 11

To further verify that insect derived miRNA sequence (ffluc1; SEQ ID NO: 9) or novel synthetic miRNA sequence (ran1g; SEQ ID NO: 17 and ran2g; SEQ ID NO: 18), have no predicted endogenous targets within the mammalian transcriptome, quantitative RT-PCR of predicted mRNA targets was performed.

Plasmids were generated that expressed the ffluc1 (SEQ ID NO: 9), ran1g (SEQ ID NO: 18) or ran2g (SEQ ID NO: 18) miRNAs from an intron downstream of the hEF1a promoter (FIG. 24A). Control plasmids were also generated that expressed the hsa-miR-132-3p, hsa-miR-34a-5p or hsa-miR-644a miRNAs from an intron downstream of the hEF1a promoter (FIG. 24B). The miRNAs expressed by the control plasmids are endogenous human miRNAs with recognised human mRNA targets (MECP2, HSPA1B and ACTB, respectively).

Human embryonic kidney 293 cells (HEK 293) were transfected with 100 μg of each plasmid using Lipofectamine®. After 48 hrs, cells were lysed and total RNA isolated. The quality and quantity of isolated RNA was analysed. First-strand synthesis was performed, in 20 μl reactions containing 500 ng of total RNA template and 500 nM random hexamers. SYBR Green PCR reactions were carried out, in 20 μl reactions using 1/10th of the first-strand synthesis reaction and 300 nM gene-specific primers. PCR was performed under the following cycling conditions: an initial denaturation at 95° C. for 3 min, then 40 cycles of 95° C. for 10 s, 55° C. for 30 s and 60° C. for 30 s, followed by a dissociation curve. Results were analysed using the 2^{-ΔΔCt} method to calculate the relative fold gene expression of samples relative to the lipofectamine-only control sample.

Quantitative RT-PCR (qRT-PCR) was used to quantify transcript levels of three of the top predicted human mRNA targets of ffluc1 (IRF2BP2, HNRNPH1 and RPP30), ran1g (FASN, ETAA1 and MAIP1) and ran2g (MCFD2, SLC38A2 and FZD6). qRT-PCR was also used to quantify transcript levels of recognised endogenous mRNA targets of miRNAs expressed by control plasmids: hsa-miR-132-3p (MECP2), hsa-miR-34a-5p (HSPA1B) or hsa-miR-644a (ACTB).

qRT-PCR assessment shows that, even when ffluc1, ran1g or ran2g are expressed at very high levels, there is minimal detectable off-target effects (FIG. 24C). The only exception is FZD6, which was robustly downregulated by ran2g. In contrast, on-target positive controls confer a profound suppression of target showing robustness of assay (FIG. 24D).

### Example 12

### In Vitro Assessment of Feed-Forward in Other CNS Indications.

The inventors identified that the present invention was also effective in treatment of other disorders affecting the central nervous system (CNS). Constructs were made, replacing MECP2 with the the UBE3A gene (mutations in this gene lead to Angelman syndrome and Prader-Willi syndrome), and the CDKL5 gene (mutations in this gene lead to CDKL5 deficiency disorder).

Plasmids were generated that expressed the ffluc1 miRNA (SEQ ID NO: 9) and a gene-of-interest (GOI), fused to a mNeonGreen reporter gene. For each GOI, a construct with and without the feedforward mechanism was generated (FIG. 25A). In regulated constructs, the 3′UTR contained three non-mammalian miRNA binding sites for the same ffluc1 miRNA used in previous experiments (SEQ ID NO: 34). In unregulated constructs the 3′UTR contained a scrambled (scr) sequence incompatible with ffluc1 miRNA binding. Human embryonic kidney 293 cells (HEK 293) were transfected with 100 μg of each plasmid using Lipofectamine 3000. After 48 hours, cells were collected, and the level of transgene expression was assessed by flow cytometry. A separate fluorescent marker on the construct (mRuby) was used to monitor the level of construct delivered to each cell (surrogate for dose).

FIGS. 25B-C illustrates that feed-forward control of dosage sensitivity can be achieved across other dosage sensitive genes. Feed-forward control was seen for both the UBE3A gene (25B) (disrupted in Angelman syndrome and Prader-Willi syndrome) and the CDKL5 gene (25C) (disrupted in CDKL5 deficiency disorder).

The expression of these proteins (UBE3A and CDKL5) is determined by NeonGreen protein levels as assessed by flow cytometry. Regulated feed-forward constructs were compared against unregulated control constructs absent of miRNA regulation (FIGS. 25A-C). Graphs show levels of mRuby (x-axis—measure of amount of plasmid to the cell and not affected by miRNA regulation) versus UBE3A-NeonGreen or CDKL5-mNeonGreen (y-axis—the protein regulated by the miRNA). Results show that ffluc1 miRNAs are effective in regulating UBE3A and CDKL5 expression in feedforward samples compared to controls as shown by the difference in slope of the linear regression lines.

As seen with MECP2, the dampening effect of the feed-forward elements increased as the amount of construct delivered increased suggesting that the control elements can mitigate toxicity without impeding expression of the gene at the therapeutic level.

### Example 13

### In Vitro Assessment of Feed-Forward in Other CNS Indications—Targeting a Codon-Optimized Transgene.

The inventors determined that a codon-optimised protein coding sequence can be utilised as the miRNA binding site. A synthetic miRNA was delivered within a gene therapy cassette to target a unique miRNA binding region created within a codon optimized protein coding sequence of a transgene, instead of targeting miRNA binding sites within the 3′UTR. The synthetic miRNA has no corresponding binding site within the mammalian genome. This approach can be particularly advantageous for larger genes, which approach the packaging capacity of a viral vector.

FIGS. 26A-B illustrates that feed-forward control of dosage sensitivity can be achieved when the miRNA binding site is in the transgene protein coding sequence. The figure shows regulation of the SYNGAP1 gene (disrupted in SYNGAP1-related intellectual disability) using this approach. Plasmids were generated that expressed a codon optimised SYNGAP1 transgene fused to a mNeonGreen reporter gene, and the synthetic syn3i miRNA (SEQ ID NO: 29 regulated construct) or no miRNA (unregulated construct) (FIG. 26A). Human embryonic kidney 293 cells (HEK 293) were transfected with 100 μg of each plasmid using Lipofectamine 3000. After 48 hrs, cells were collected, and the level of transgene expression was assessed by flow cytometry. A separate fluorescent marker on the construct (mCherry) was used to monitor the level of construct delivered to each cell (surrogate for dose).

The expression of SynGAP protein was determined by NeonGreen protein levels as assessed by flow cytometry. Regulated feed-forward constructs were compared against unregulated control constructs absent of miRNA regulation (FIGS. 26A-B). Graphs show levels of mRuby (x-axis—measure of amount of plasmid to the cell and not affected by miRNA regulation) versus SynGAP-NeonGreen (y-axis—the protein regulated by the miRNA). Results show that syn3i miRNAs (SEQ ID NO: 29) are effective in regulating SynGAP expression in feedforward samples compared to controls as shown by the difference in slope of the linear regression lines.

The dampening effect of the feed-forward elements increased as the amount of construct delivered increased suggesting that this alternative embodiment of the feed-forward principle can also mitigate toxicity without impeding expression of the gene at the therapeutic level.

### Example 14

### In Vitro Assessment of Feed-Forward in Other Non-CNS Indications

The non-mammalian miRNA feedforward mechanism was also effective in other disorders where the primary phenotype is peripheral rather than the central nervous system (CNS). Constructs were made with MECP2 replaced with the coding sequence for other proteins: the SMN1 gene (mutations in this gene lead to spinal muscular atrophy), the INS gene (mutations in this gene lead to type 1 diabetes) and the FXN gene (mutations in this gene lead to Friedreich's ataxia). The 3′UTR contained 3 non-mammalian miRNA binding sites for the same ffluc1 miRNA (SEQ ID NO: 9) used in previous experiments (SEQ ID NO: 34).

Plasmids were generated that expressed the ffluc1 miRNA and one of the genes-of-interest (GOI) above. The GOI was fused to a mNeonGreen reporter gene. For each GOI, a construct with and without the feedforward mechanism was generated (FIG. 27A). In regulated constructs, the 3′UTR contained the SEQ ID NO: 34 miRNA binding site. In unregulated constructs, the 3′UTR contained a scrambled (scr) sequence incompatible with ffluc1 miRNA binding. Human embryonic kidney 293 cells (HEK 293) were transfected with 100 μg of each plasmid using Lipofectamine 3000. After 48 hours, cells were collected, and the level of transgene expression was assessed by flow cytometry. A separate fluorescent marker on the construct (mRuby) was used to monitor the level of construct delivered to each cell (surrogate for dose).

FIGS. 27B-D illustrates feed-forward control of dosage sensitivity can be achieved across other dosage sensitive genes, in this case (27B) the SMN1 gene disrupted in spinal muscular atrophy, (27C) the INS gene disrupted in type 1 diabetes, (27D) the FXN gene disrupted in Friedreich's ataxia.

The expression of these proteins (SMN1, insulin and Frataxin) are determined by NeonGreen protein levels as assessed by flow cytometry. Regulated feed-forward constructs were compared against unregulated control constructs absent of miRNA regulation (FIGS. 27A-D). Graphs show levels of mRuby (x-axis—measure of amount of plasmid to the cell and not affected by miRNA regulation) versus SMN1-NeonGreen, insulin-mNeonGreen or Frataxin-mNeonGreen (y-axis—the protein regulated by the miRNA). Results show that ffluc1 miRNAs are effective in regulating SMN1, insulin and Frataxin expression in feedforward samples compared to controls as shown by the difference in slope of the linear regression lines.

As seen with MECP2, the dampening effect of the feed-forward elements increased as the amount of construct delivered increased suggesting that the control elements can mitigate toxicity without impeding expression of the gene at the therapeutic level.

### Example 15

### UBE3A Regulation In Vivo

The inventors further demonstrated the use of a non-mammalian miRNA feedforward mechanism in treating other dosage sensitive disorders which affect the central nervous system (CNS). The UBE3A gene, disrupted in Angelman syndrome and Prader-Willi syndrome, was shown to be regulated in vivo by the feedforward mechanism.

Constructs were generated that expressed the ffluc1 miRNA (SEQ ID NO: 9) and human UBE3A, fused to a 3×FLAG tag. A construct with and without the feedforward mechanism was generated (FIG. 28A). In regulated constructs the 3′UTR contained miRNA binding site SEQ ID NO: 34. Single-stranded AAV (ssAAV) particles of the regulated and unregulated UBE3A constructs, comprising constructs flanked by AAV2 ITRs packaged into AAV9 capsids, were produced by a baculovirus transfection system at Virovek (Hayward, CA, USA).

FIG. 28B demonstrates that a UBE3A regulated feed forward cassette provides regulation in vivo when compared to an unregulated UBE3A cassette. Immunoblot analysis using an anti-FLAG antibody provides a readout of UBE3A expression levels in cells. AAV vectors expressing feed-forward UBE3A constructs were tested in wild-type mice maintained on a mixed CBA/C57 background. ssAAV expressing regulated or unregulated UBE3A was injected bilaterally into the brains of postnatal day (P)1 males by intracerebroventricular (ICV) administration. Control injections used PBS (vehicle control). Injected pups were culled 7 days post-injection and tissues collected for analysis. Fresh tissue samples were homogenised in a bead mill with 300 μl of buffer NE1 then stored on ice. After addition of 250 U benzonase nuclease to each sample, samples were shaken, incubated at room temperature for 15 minutes, then stored on ice. Samples were diluted 1:20 in NE1 buffer for protein quantification. 100 μl 4×Laemmli Sample Buffer was added to each bead mill tube, samples boiled for 10 min, then stored at −80° C. Samples were thawed and 25 μg amount of each sample migrated on a 10% acrylamide gel at 150 V until the dye front reached the bottom of the gel. Gels were then transferred to a nitrocellulose membrane for 2 hours at 85 V. Total protein was measured. Total protein stain was removed, and membranes were incubated with LI-COR® blocking buffer on a shaking incubator for 1 hour at room temperature. Membranes were then incubated overnight at 4° C. in 20 ml of LI-COR® blocking buffer with primary antibody anti-FLAG at a dilution of 1:2000. Membranes were washed in TBS-T buffer for 10 minutes (×3), and then incubated for 2 hours at room temperature in 20 ml of LI-COR® blocking buffer with secondary antibody IRDye 800CW Donkey anti-mouse at a dilution of 1:10000. Membranes were washed in TBS-T buffer for 10 minutes (×3), rinsed with TBS buffer then imaged.

This demonstrates that UBE3A (i.e., transgenes other than MECP2) can be regulated in vivo under control of the non-mammalian miRNA feedforward mechanism. This reduces the probability of tissue damage/toxicity from overexpression of transgenes where the gene/disorder is known to be dosage sensitive.

### Example 16

Feed-Forward Constructs Package Efficiently in ssAAV

Feed-forward constructs expressing the MECP2 transgene were prepared as single-stranded AAV (ssAAV) particles, comprising constructs flanked by AAV2 ITRs packaged into AAV9 capsids and were produced either by a HEK293 process (Viral Vector Production Unit, Universitat Autonoma Barcelona, Spain) or by a baculovirus based infection system at Virovek (Hayward, CA, USA). Using both processes, the inventors demonstrate that the feed-forward gene therapy constructs can be produced efficiently, to scale and to very high titer (up to 1.94×10¹⁴ viral genomes/ml). Therefore, the inventors have identified that the feed-forward regulated gene therapy technology has been configured for efficient manufacture. Importantly, the inventors demonstrate that the feed-forward synthetic circuit constructs package efficiently in AAV.

Following AAV production, CDMS (charge detection mass spectrometry) analysis was performed to determine the size of the AAV particles based on charge and mass. This tool helps in determining the quality of packaging and if there are any partially packaged species that might potentially affect the potency of the AAV product.

FIG. 29 depicts a representative CDMS analysis of a feed-forward MECP2 construct packaged in ssAAV9. Full-length feed-forward products package as expected, with low levels of aberrant or partial packaging. Secondary DNA structures, such as hairpins, are known to inhibit efficient packaging in AAV particles. However, in the feed-forward constructs analysed, the presence of miRNA hairpins (in the EF1a or MINIX intron) do not cause significant packaging of smaller than expected/partially packaged particles, and do not affect the quality of the AAV preparation.

It is known that genetic sequence containing secondary structure such as stem loops, hairpins and miRNA generating sequence very commonly result in aberrant packaging and the encapsulation of heterogeneous species that adversely compromise product purity (Xie et al., 2017). FIG. 29 displays a profile which is considered a very clean profile within the state of the art. The inventors have, therefore, provided a solution to the purity issue, by developing a feed-forward AAV construct which is capable of large-scale manufacture into a high-purity product.

1. A construct comprising:
a promoter;
at least one non-mammalian or synthetic miRNA is expressed within an intron, wherein the synthetic miRNA is a sequence which is not naturally occurring;
a transgene;
at least one non-mammalian or synthetic miRNA binding site(s) which provides for control of the expression of the transgene, wherein the synthetic miRNA binding site(s) is a sequence which is not naturally occurring; and
a polyadenylation signal.

2. The construct of claim 1, wherein the miRNA binding site(s) which provide for control of the expression of the transgene is provided within the 3′ UTR, or the 5′ UTR.

3. The construct of claim 1, wherein the non-mammalian or synthetic miRNA binding site(s) is provided within the transgene.

4. The construct of claim 1, wherein the construct provides a single gene circuit to provide a relatively fixed level of expression of the transgene across cells receiving different levels of vector-derived transgene dosage-insensitivity).

5. The construct of claim 1, wherein the at least one synthetic or non-mammalian miRNA exhibits no off-target binding effects.

6. The construct of claim 1, wherein the non-mammalian or synthetic miRNA is expressed in an intron provided by SEQ ID NO: 5 or SEQ ID NO: 6.

7. The construct of claim 1, wherein the miRNA is non-mammalian miRNA derived from an insect miRNA, optionally wherein the miRNA is capable of specifically binding to firefly lucifersase (ffluc1) miRNA binding site.

8. The construct of claim 1, wherein there are a plurality of miRNA binding sites provided in the construct, optionally three miRNA binding sites, at least four miRNA binding sites, at least five miRNA binding sites, or at least six miRNA binding sites.

9. The construct of claim 1, wherein there are a plurality of non-mammalian or synthetic miRNAs expressed in a construct.

10. The construct of claim 1, wherein the non-mammalian firefly luciferase miRNA is a sequence selected from SEQ ID NO: 9-12.

11. The construct of claim 1, wherein the synthetic miRNA is a sequence selected from SEQ ID NO: 13-20.

12. The construct of claim 1, wherein the synthetic miRNA is targeted against the coding sequence of a target gene and is selected from SEQ ID NO: 21-32.

13. The construct of claim 1, wherein the non-mammalian or synthetic miRNA binding site is selected from SEQ ID NO: 33-55.

14. The construct of claim 12, wherein the synthetic miRNA is targeted against the coding sequence of a target gene and the synthetic miRNA binding site is selected from SEQ ID NO: 56-67.

15. The construct of claim 1, wherein the promoter is selected from a constitutive or conditional promoter, optionally wherein the promoter is tissue specific.

16. The construct of claim 1, wherein the promoter is selected from SEQ ID NO 68 or SEQ ID NO: 69.

17. The construct of claim 1, wherein the polyA sequence is selected from SEQ ID NO: 70-72.

18. The construct of claim 1, further comprising a stability element, wherein the stability element is located in the 3′UTR.

19. The construct of claim 18, wherein the stability element is selected from SEQ ID NO: 74 or SEQ ID NO: 75.

20. The construct of claim 1, wherein the construct further comprises the Kozak sequence GCCACCATGG (SEQ ID NO: 73).

21. The construct of claim 1, wherein the miRNA binding site has been designed to partially ameliorate miRNA binding.

22. A vector comprising a construct of any one of claim 1.

23. The vector of claim 22, wherein the vector is an AAV or lentiviral vector, optionally wherein the vector is an AAV vector, optionally wherein the construct is operably linked to expression control elements, and the expression control elements and the construct are together flanked by 5′ and 3′ AAV inverted terminal repeats (ITR).

24. The vector of claim 22, packaged into a virion, optionally wherein the vector when packaged into the viron virion does not affect the quality of the construct.

25. The vector of claim 22, formulated in a nanoparticle.

26. A method of using the construct of claim 1 to express a transgene, optionally to express a transgene is a specific mammalian cell type or types.

27. A method of treating a disorder in a subject, the method comprising the step of providing the construct of claim 1.

28. (canceled)

29. The method of claim 27, wherein the disorder is any monogenic disorder in which controlled expression of the corrective gene is desired, optionally wherein the monogenic disorder is selected from the group consisting of Rett Syndrome, Fragile X syndrome, Angelman syndrome, Syngap-related intellectual disability, CDK15 deficiency, Fredrich's ataxia, Spinal muscular dystrophy, Haemophilia, and Diabetes.

30. The method of claim 27, wherein the disorder is treated by expression of a gene selected from the list comprising: PRKCZ, TTC34, PRDM16, ARHGEF16, PARK7, PRDM2, IGSF21, PTCH2, NFIA, ST6GALNAC3, DPYD, COL11A1, PDZK1, GPR89A, NBPF11, GPR89B, KCNT2, CFHR2, ASPM, PTPRC, GPATCH2, DUSP10, GPR137B, RYR2, CHRM3, RGS7, AKT3, KIF26B, SMYD3, LPIN1, EPCAM, MSH2, NRXN1, XPO1, LRP1B, ZEB2, ACVR2A, MBD5, KIF5C, SCN1A, COL3A1, PMS1, PLCL1, SATB2, PARD3B, EPHA4, SPHKAP, CHL1, GRM7, TRANK1, DOCK3, FAM19A1, FOXP1, ROBO1, CADM2, FOXL2, SOX2, LPP, RASGEF1B, GRID2, FAT4, NR3C2, LRBA, FGA, GALNTL6, WWC2, TLR3, IRX2, IRX1, CDH12, CDH9, NIPBL, HEXB, MEF2C, GRAMD3, FBN2, PRELID2, TCOF1, GABRG2, MSX2, NSD1, FOXC1, CDYL, TBC1D7, RUNX2, MUT, RIMS1, NKAIN2, LAMA2, ARID1B, PARK2, PACRG, QKI, TNRC18, FBXL18, SUGCT, GLI3, AUTS2, MLXIPL, COL1A2, PPP1R9A, CFTR, TSPAN12, GRM8, CNTNAP2, MNX1, CSMD1, MCPH1, LPL, ANK1, IMPAD1, CHD7, VCPIP1, TRPS1, PARP10, DOCKS, KANK1, GLIS3, PTPRD, MLLT3, ROR2, PTCH1, AL162389.1, ARRDC1, EHMT1, PCDH15, CTNNA3, ADK, BMPR1A, PAX2, BTRC, INPP5A, MRPL23, ELP4, PAX6, CPT1A, DYNC2H1, KIRREL3, WNK1, CACNA1C, PPFIBP1, TBX5, MED13L, NALCN, CHD8, MYH7, TTC6, DAAM1, NRXN3, MTA1, SNRPN, UBE3A, OCA2, HERC2, CHRFAM7A, ARHGAP11B, OTUD7A, FBN1, HEXA, SNUPN, NRG4, AC112693.2, IGF1R, LRRC28, HBA2, HBQ1, CREBBP, RBFOX1, CDR2, CDH13, CYBA, NXN, YWHAE, SMG6, METTL16, PAFAH1B1, ADORA2B, NT5M, RAIL NF1, C17orf67, PITPNC1, ACOX1, TCF4, DOCK6, CACNA1A, LPHN1, ZSCAN5A, BMP2, MYT1, PEX26, USP18, DGCR6L, USP41, UBE2L3, NF2, LARGE, BRD1, SHANK3 CDKL5, FXN, SMN1, F8, and INS.
